(19) United States
(12) Patent Application Publication
(10) Pub. No.: US 2026/0184704 A1
(43) Pub. Date: Jul. 2, 2026

(54) NLRP3 INFLAMMASOME INHIBITORS AND USES THEREOF

(71) Applicant: Insilico Medicine IP Limited, Hong Kong (CN)

(72) Inventors: Jianping WU, Shanghai (CN); Luoheng QIN, Shanghai (CN); Xiaoyu DING, Shanghai (CN); Feng REN, Shanghai (CN); Chaopeng LI, Shanghai (CN)

(21) Appl. No.: 19/441,614

(22) Filed: Jan. 6, 2026

Related U.S. Application Data
(63) Continuation of application No. 19/164,556.

(30) Foreign Application Priority Data
Mar. 20, 2023 (WO) PCT/CN2023/082488
Jun. 19, 2023 (WO) PCT/CN2023/101049
Mar. 1, 2024 (WO) PCT/CN2024/079655

(51) Int. Cl. C07D 417/12; A61K 31/5395; A61K 31/549; C07D 285/16; C07D 413/12; C07D 417/04; C07D 417/14; C07D 453/02; C07D 471/04

(52) U.S. Cl. CPC C07D 417/12; A61K 31/5395; A61K 31/549; C07D 285/16; C07D 413/12; C07D 417/04; C07D 417/14; C07D 453/02; C07D 471/04

(57) ABSTRACT

Described herein are NLRP3 inflammasome inhibitors and pharmaceutical compositions comprising said inhibitors. The subject compounds and compositions are useful for the treatment of a disease or disorder associated with NLRP3 inflammasome pathway.

### CROSS REFERENCE TO RELATED APPLICATIONS

This patent application is a continuation application of U.S. application Ser. No. 19/164,556, filed Sep. 11, 2025, which is a National Stage Entry of International Application No. PCT/CN2024/082447, filed Mar. 19, 2024, which claims the benefit of International Application No. PCT/CN2023/082488, filed Mar. 20, 2023, International Application No. PCT/CN2023/101049, filed Jun. 19, 2023, and International Application No. PCT/CN2024/079655, filed Mar. 1, 2024, each of which is incorporated herein by reference in its entirety.

### BACKGROUND

NOD-like receptor (NLR) family, pyrin domain-containing protein 3 (NLRP3) is an intracellular sensor that detects a broad range of microbial motifs, endogenous danger signals and environmental irritants, resulting in the formation and activation of the NLRP3 inflammasome. Assembly of the NLRP3 inflammasome leads to caspase 1-dependent release of the pro-inflammatory cytokines IL-1β and IL-18, as well as to gasdermin D-mediated pyroptotic cell death. Studies have revealed new regulators of the NLRP3 inflammasome, including new interacting or regulatory proteins, metabolic pathways, and a regulatory mitochondrial hub. The aberrant activation of the NLRP3 inflammasome has been linked with several inflammatory disorders, which include cryopyrin-associated periodic syndromes, Alzheimer's disease, diabetes, and atherosclerosis.

Based on the foregoing, there is a need to identify inhibitors of NLRP3 inflammasome.

### SUMMARY

In one aspect, the present disclosure relates to novel compounds and compositions that are useful as inhibitors of NLRP3 inflammasome pathway.

In one aspect, the disclosure provides for a compound represented by Formula (I), or a pharmaceutically acceptable salt or a stereoisomer thereof:

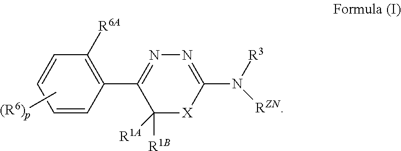

In some embodiments, the disclosure provides for a compound represented by Formula (Ia), or a pharmaceutically acceptable salt or a stereoisomer thereof:

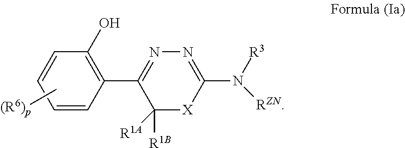

In some embodiments of a compound of Formula (Ia), is a compound is of Formula (II), or a pharmaceutically acceptable salt or a stereoisomer thereof:

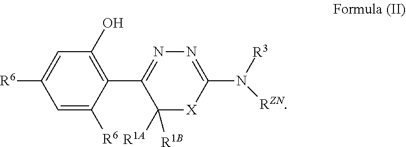

In another aspect, provided herein is a compound represented by Formula (III), or a pharmaceutically acceptable salt or a stereoisomer thereof:

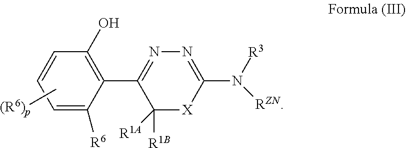

In another aspect, provided herein is a compound represented by Formula (IV), or a pharmaceutically acceptable salt or a stereoisomer thereof:

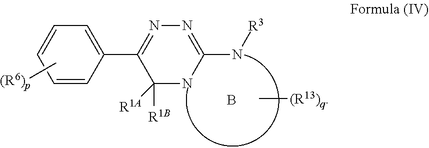

Also disclosed herein is a pharmaceutical composition comprising a compound disclosed herein, or a pharmaceutically acceptable salt or a stereoisomer thereof, and a pharmaceutically acceptable excipient.

Also disclosed herein is a method of modulating NLRP3 inflammasome in a subject, the method comprising administering to the subject the compound disclosed herein, or a pharmaceutically acceptable salt or a stereoisomer thereof.

Also disclosed herein is a method of inhibiting NLRP3 in a subject, the method comprising administering to the subject the compound disclosed herein, or a pharmaceutically acceptable salt or a stereoisomer thereof.

Also disclosed herein is a method of treating an auto-immune or auto-inflammatory disease or condition in a subject in need thereof, the method comprising administering to the subject a compound disclosed herein, or a pharmaceutically acceptable salt or a stereoisomer thereof.

In some embodiments, the disease or disorder is selected from inflammasome-related diseases/disorders, immune diseases, inflammatory diseases, auto-immune diseases, or auto-inflammatory diseases, for example, autoinflammatory fever syndromes (e.g., cryopyrin-associated periodic syndrome), liver related diseases/disorders (e.g. chronic liver disease, viral hepatitis, non-alcoholic steatohepatitis (NASH), alcoholic steatohepatitis, and alcoholic liver disease), inflammatory arthritis related disorders (e.g. gout, pseudogout (chondrocalcinosis), osteoarthritis, rheumatoid arthritis, arthropathy e.g., acute, chronic), kidney related diseases (e.g. hyperoxaluria, lupus nephritis, Type I/Type II diabetes and related complications (e.g. nephropathy, retinopathy), hypertensive nephropathy, hemodialysis related inflammation), neuroinflammation-related diseases (e.g. multiple sclerosis, brain infection, acute injury, neurodegenerative diseases, Alzheimer's disease), cardiovascular/metabolic diseases/disorders (e.g. cardiovascular risk reduction (CvRR), hypertension, atherosclerosis, type I and type II diabetes and related complications, peripheral artery disease (PAD), acute heart failure), inflammatory skin diseases (e.g. hidradenitis suppurativa, acne), wound healing and scar formation, asthma, sarcoidosis, age-related macular degeneration, and cancer related diseases/disorders (e.g. colon cancer, lung cancer, myeloproliferative neoplasms, leukemias, myelodysplastic syndromes (MDS), myelofibrosis).

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Definitions

Unless defined otherwise, all technical and scientific terms used herein have the same meaning as is commonly understood by one of skill in the art to which this invention belongs. All patents and publications referred to herein are incorporated by reference.

Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“oxo” refers to ═O.

“Carboxyl” refers to-COOH.

“Cyano” refers to-CN.

“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀alkyl. In some embodiments, the alkyl is a C₁₋₆alkyl. In some embodiments, the alkyl is a C₁₋₅alkyl. In some embodiments, the alkyl is a C₁₋₄alkyl. In some embodiments, the alkyl is a C₁₋₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COOH, COOMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —COOH, COOMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, COOMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system can contain only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, COOMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Carbocycle” refers to a saturated, unsaturated, or aromatic rings in which each atom of the ring is carbon. Carbocycle may include 3- to 10-membered monocyclic rings, 6- to 12-membered bicyclic rings, and 6- to 12-membered bridged rings. Each ring of a bicyclic carbocycle may be selected from saturated, unsaturated, and aromatic rings. An aromatic ring, e.g., phenyl, may be fused to a saturated or unsaturated ring, e.g., cyclohexane, cyclopentane, or cyclohexene. Any combination of saturated, unsaturated, and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Unless stated otherwise specifically in the specification, a carbocycle may be optionally substituted.

“Cycloalkyl” refers to a partially or fully saturated, monocyclic, or polycyclic carbocyclic ring, which may include fused (when fused with an aryl or a heteroaryl ring, the cycloalkyl is bonded through a non-aromatic ring atom), spiro, or bridged ring systems. In some embodiments, the cycloalkyl is fully saturated. Representative cycloalkyls include, but are not limited to, cycloalkyls having from three to fifteen carbon atoms (e.g., C₃-C₁₅ fully saturated cycloalkyl or C₃-C₁₅ cycloalkenyl), from three to ten carbon atoms (e.g., C₃-C₁₀ fully saturated cycloalkyl or C₃-C₁₀ cycloalkenyl), from three to eight carbon atoms (e.g., C₃-C₈ fully saturated cycloalkyl or C₃-C₈ cycloalkenyl), from three to six carbon atoms (e.g., C₃-C₆ fully saturated cycloalkyl or C₃-C₆ cycloalkenyl), from three to five carbon atoms (e.g., C₃-C₅ fully saturated cycloalkyl or C₃-C₅ cycloalkenyl), or three to four carbon atoms (e.g., C₃-C₄ fully saturated cycloalkyl or C₃-C₄ cycloalkenyl). In some embodiments, the cycloalkyl is a 3- to 10-membered fully saturated cycloalkyl or a 3- to 10-membered cycloalkenyl. In some embodiments, the cycloalkyl is a 3- to 6-membered fully saturated cycloalkyl or a 3- to 6-membered cycloalkenyl. In some embodiments, the cycloalkyl is a 5- to 6-membered fully saturated cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, COOMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Cycloalkenyl” refers to an unsaturated non-aromatic monocyclic or polycyclic hydrocarbon radical consisting solely of carbon and hydrogen atoms, which includes fused or bridged ring systems, preferably having from three to twelve carbon atoms and comprising at least one double bond. In certain embodiments, a cycloalkenyl comprises three to ten carbon atoms. In other embodiments, a cycloalkenyl comprises five to seven carbon atoms. The cycloalkenyl may be attached to the rest of the molecule by a single bond. Examples of monocyclic cycloalkenyls includes, e.g., cyclopentenyl, cyclohexenyl, cycloheptenyl, and cyclooctenyl.

“Halo” or “halogen” refers to bromo, chloro, fluoro or iodo. In some embodiments, halogen is fluoro or chloro. In some embodiments, halogen is fluoro.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., Cl, Br, F, I, etc.). When an alkyl group is substituted with more than one halogen radicals, each halogen may be independently selected e.g., 1-chloro,2-fluoroethane.

“Fluoroalkyl” refers to an alkyl radical, as defined above, that is substituted by one or more fluoro radicals, for example, trifluoromethyl, difluoromethyl, fluoromethyl, 2,2,2-trifluoroethyl, 1-fluoromethyl-2-fluoroethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous, silicon, and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom), spiro, or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (e.g., C₂-C₁₅ fully saturated heterocycloalkyl or C₂-C₁₅ heterocycloalkenyl), from two to ten carbon atoms (e.g., C₂-C₁₀ fully saturated heterocycloalkyl or C₂-C₁₀ heterocycloalkenyl), from two to eight carbon atoms (e.g., C₂-C₈ fully saturated heterocycloalkyl or C₂-C₈ heterocycloalkenyl), from two to seven carbon atoms (e.g., C₂-C₇ fully saturated heterocycloalkyl or C₂-C₇ heterocycloalkenyl), from two to six carbon atoms (e.g., C₂-C₆ fully saturated heterocycloalkyl or C₂-C₆ heterocycloalkenyl), from two to five carbon atoms (e.g., C₂-C₅ fully saturated heterocycloalkyl or C₂-C₅ heterocycloalkenyl), or two to four carbon atoms (e.g., C₂-C₄ fully saturated heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides, and the oligosaccharides. In some embodiments, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered fully saturated heterocycloalkyl. In some embodiments, the heterocycloalkyl is a 3- to 7-membered fully saturated heterocycloalkyl. In some embodiments, the heterocycloalkyl is a 3- to 6-membered fully saturated heterocycloalkyl. In some embodiments, the heterocycloalkyl is a 4- to 6-membered fully saturated heterocycloalkyl. In some embodiments, the heterocycloalkyl is a 5- to 6-membered fully saturated heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —COOH, COOMe, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “substituted” refers to moieties having substituents replacing a hydrogen on one or more carbons or substitutable heteroatoms, e.g., NH, of the structure. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched, and unbranched, carbocyclic, and heterocyclic, aromatic, and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

The term “one or more” when referring to an optional substituent means that the subject group is optionally substituted with one, two, three, or four substituents. In some embodiments, the subject group is optionally substituted with one, two, or three substituents. In some embodiments, the subject group is optionally substituted with one or two substituents. In some embodiments, the subject group is optionally substituted with one substituent. In some embodiments, the subject group is optionally substituted with two substituents.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition, or vehicle, such as a liquid or solid filler, diluent, excipient, solvent, or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

An “effective amount” or “therapeutically effective amount” refers to an amount of a compound administered to a mammalian subject, either as a single dose or as part of a series of doses, which is effective to produce a desired therapeutic effect.

The terms “treat,” “treating” or “treatment,” as used herein, include alleviating, abating, or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition.

As used herein, a “disease or disorder associated with NLRP3 inflammasome” or, alternatively, “a NLRP3 inflammasome-mediated disease or disorder” means any disease or other deleterious condition in which the NLRP3 inflammasome is known or suspected to play a role.

### Compounds of the Disclosure

Described herein are compounds, or a pharmaceutically acceptable salt thereof useful in the treatment of a disease or disorder associated with NLRP3 inflammasome.

In one aspect, the disclosure provides a compound represented by Formula (A) or Formula (B), or a pharmaceutically acceptable salt or a stereoisomer thereof:

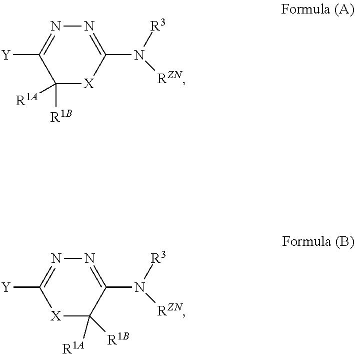

wherein;
Y is C₃-C₈ cycloalkyl, 3- to 8-membered heterocycloalkyl, C₆-Cie aryl, or 5- to 9-membered heteroaryl, wherein the C₃-C₈ cycloalkyl, 3- to 8-membered heterocycloalkyl, C₆-C₁₀ aryl, or 5- to 9-membered heteroaryl is optionally substituted with one or more R⁶;
X is NR^{X}, —O—, —S—, —S(O)—, or —S(O)₂—;
R^{X} is hydrogen, C₁-C₆alkyl, C₁-C₆haloalkyl, C₁-C₆heteroalkyl, C₁-C₆hydroxyalkyl, C₁-C₆aminoalkyl, C₃-C₆cycloalkyl, or 4 to 6 membered heterocycloalkyl; wherein each of the alkyl, haloalkyl, hydroxyalkyl, heteroalkyl, aminoalkyl, cycloalkyl, or heterocycloalkyl is optionally substituted with 1 to 4 substituents independently selected from R^{e};
each R^{1A} and R^{1B} is independently hydrogen, halogen, —CN, —NO₂, —OH, —OR^{a}, —NR^{c}R^{d}, C₁-C₆alkyl, C₁-C₆haloalkyl, C₁-C₆hydroxyalkyl, or C₁-C₆aminoalkyl;
or R^{1A} and R^{1B} are taken together to form an oxo;
or R^{1A} and R^{1B} are taken together to form a C₃-C₈acycloalkyl or 4 to 8 membered heterocycloalkyl; each of which is optionally substituted with one or more R¹¹;
each R¹¹ is independently halogen, —OH, —CN, —NO₂, —OR^{a}, —NR^{c}R^{d}, C₁-C₆alkyl, C₁-C₆alkoxy, C₁-C₆haloalkyl, C₁-C₆hydroxyalkyl, C₁-C₆aminoalkyl, C₁-C₆heteroalkyl, C₃-C₆cycloalkyl, or 4 to 6 membered heterocycloalkyl;
R³ is phenyl, 5 to 12 membered heteroaryl, C₃-C₁₂cycloalkyl, 4 to 12 membered heterocycloalkyl, or C₁-C₆alkyl; each of which is optionally substituted with one or more R⁸;
each R⁸ is independently halogen, —OH, —CN, —NO₂, —OR^{a}, —OC(═O)R^{a}, —OC(═O)OR^{b}, —OC(═O)NR^{c}R^{d}, —SH, —SR^{a}, SF₅, —S(═O)R^{a}, —S(═O)₂R^{a}, —S(═O)(═NR^{b})R^{a}, —S(═O)₂NR^{c}R⁴, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)R^{c}R^{d}, —P(═O)R^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆heteroalkyl, C₁-C₆aminoalkyl, C₃-C₆cycloalkyl, C₆-C₁₀ aryl, or 4 to 6 membered heterocycloalkyl, wherein each of the alkyl, haloalkyl, hydroxyalkyl, heteroalkyl, aminoalkyl, cycloalkyl, aryl, or heterocycloalkyl is optionally substituted with 1 to 4 substituents independently selected from R^{e};
R^{ZN} is hydrogen, C₁-C₆alkyl, or C₁-C₆haloalkyl;
or R^{X} and R^{ZN} together with the atoms to which they are attached form a 5 to 8 membered heterocycloalkyl which is optionally substituted with one or more R¹³;
or R³ and R^{ZN} together with the atoms to which they are attached form a 5 to 13 membered heterocycloalkyl which is optionally substituted with one or more R¹³;
each R¹³ is independently halogen, —OH, —CN, —NO₂, —OR^{a}, —NR^{c}R^{d}, C₁-C₆alkyl, C₁-C₆alkoxy, C₁-C₆haloalkyl, C₁-C₆hydroxyalkyl, or C₁-C₆aminoalkyl;
each R⁶ is independently halogen, —CN, —NO₂, —OH, —OR^{a}, —SH, —SR^{a}, —SF₅, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, or C₃-C₈cycloalkyl, wherein each of the alkyl, haloalkyl, hydroxyalkyl, heteroalkyl, aminoalkyl, alkenyl alkynyl, or cycloalkyl is optionally substituted with 1 to 4 substituents independently selected from R^{e};
or two R⁶ are taken together with the atoms to which they are attached to form an aryl, heteroaryl, cycloalkyl or heterocycloalkyl, each of which is optionally substituted with one or more R¹²;
each R¹² is independently halogen, —OH, —CN, —NO₂, —OR^{a}, —NR^{c}R^{d}, C₁-C₆alkyl, C₁-C₆alkoxy, C₁-C₆haloalkyl, C₁-C₆hydroxyalkyl, C₁-C₆aminoalkyl, C₁-C₆heteroalkyl, C₃-C₆cycloalkyl, or 4 to 6 membered heterocycloalkyl;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
or R^{c} and R^{d} are taken together with the atom to which they are attached to form a heterocycloalkyl, wherein the heterocycloalkyl is optionally substituted with 1 to 4 substituents independently selected from R^{e}; and
each R^{e} is independently halogen, oxo, —CN, —OH, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, C₁-C₆heteroalkyl, or C₃-C₆cycloalkyl.

In some embodiments of Formula (A), Y is C₃-C₈ cycloalkyl or 3- to 8-membered heterocycloalkyl, wherein the C₃-C₈ cycloalkyl or 3- to 8-membered heterocycloalkyl is optionally substituted with one or more R⁶. In some embodiments of Formula (A), Y is C₆-C₁₀ aryl or 5- to 9-membered heteroaryl, wherein the C₆-C₁₀ aryl or 5- to 9-membered heteroaryl is optionally substituted with one or more R⁶. In some embodiments of Formula (A), Y is 5- to 9-membered heteroaryl. In some embodiments of Formula (A), Y is C₆-C₁₀ aryl. In some embodiments of Formula (A), Y is phenyl.

In another aspect, the disclosure provides a compound represented by Formula (I) or Formula (V), or a pharmaceutically acceptable salt or a stereoisomer thereof:

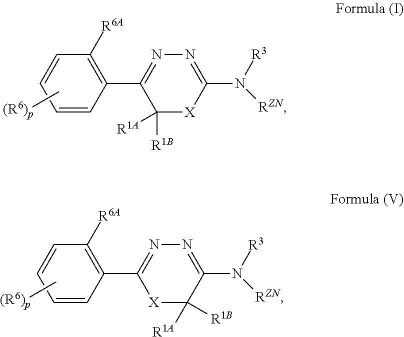

wherein;
X is NR^{X}, —O—, —S—, —S(O)—, or —S(O)₂—;
R^{X} is hydrogen, C₁-C₆alkyl, C₁-C₆haloalkyl, C₁-C₆heteroalkyl, C₁-C₆hydroxyalkyl, C₁-C₆aminoalkyl, C₃-C₆cycloalkyl, or 4 to 6 membered heterocycloalkyl; wherein each of the alkyl, haloalkyl, hydroxyalkyl, heteroalkyl, aminoalkyl, cycloalkyl, or heterocycloalkyl is optionally substituted with 1 to 4 substituents independently selected from R^{e};
each R^{1A} and R^{1B} is independently hydrogen, halogen, —CN, —NO₂, —OH, —OR^{a}, —NR^{c}R^{d}, C₁-C₆alkyl, C₁-C₆haloalkyl, C₁-C₆hydroxyalkyl, or C₁-C₆aminoalkyl;
or R^{1A} and R^{1B} are taken together to form an oxo;
or R^{1A} and R^{1B} are taken together to form a C₃-C₈cycloalkyl or 4 to 8 membered heterocycloalkyl, each of which is optionally substituted with one or more R¹¹;
each R¹¹ is independently halogen, —OH, —CN, —NO₂, —OR^{a}, —NR^{c}R^{d}, C₁-C₆alkyl, C₁-C₆alkoxy, C₁-C₆haloalkyl, C₁-C₆hydroxyalkyl, C₁-C₆aminoalkyl, C₁-C₆heteroalkyl, C₃-C₆cycloalkyl, or 4 to 6 membered heterocycloalkyl;
R³ is phenyl, 5 to 12 membered heteroaryl, C₃-C₁₂cycloalkyl, 4 to 12 membered heterocycloalkyl, or C₁-C₆alkyl; each of which is optionally substituted with one or more R⁸;
each R⁸ is independently halogen, —OH, —CN, —NO₂, —OR^{a}, —OC(═O)R^{a}, —OC(═O)OR^{b}, —OC(═O)NR^{c}R^{d}, —SH, —SR^{a}, SF₅, —S(═O)R^{a}, —S(═O)₂R^{a}, —S(═O)(═NR^{b})R^{a}, —S(═O)₂NR^{c}R⁴, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)R^{c}R^{d}, —P(═O)R^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆heteroalkyl, C₁-C₆aminoalkyl, C₃-C₆cycloalkyl, or 4 to 6 membered heterocycloalkyl, wherein each of the alkyl, haloalkyl, hydroxyalkyl, heteroalkyl, aminoalkyl, cycloalkyl, or heterocycloalkyl is optionally substituted with 1 to 4 substituents independently selected from R^{e};
R^{ZN} is hydrogen, C₁-C₆alkyl, or C₁-C₆haloalkyl;
or R^{X} and R^{ZN} together with the atoms to which they are attached form a 4 to 8 membered ring which is optionally substituted with one or more R¹³;
each R¹³ is independently halogen, —OH, —CN, —NO₂, —OR^{a}, —NR^{c}R^{d}, C₁-C₆alkyl, C₁-C₆alkoxy, C₁-C₆haloalkyl, C₁-C₆hydroxyalkyl, or C₁-C₆aminoalkyl;
R^{6A} is —OH, —OCF₂H, —CF₂H, or —CF₃;
each R⁶ is independently halogen, —CN, —NO₂, —OH, —OR^{a}, —SH, —SR^{a}, —SF₅, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, or C₃-C₈cycloalkyl, wherein each of the alkyl, haloalkyl, hydroxyalkyl, heteroalkyl, aminoalkyl, alkenyl alkynyl, or cycloalkyl is optionally substituted with 1 to 4 substituents independently selected from R^{e};
or two R⁶ are taken together with the atoms to which they are attached to form an aryl, heteroaryl, cycloalkyl or heterocycloalkyl, each of which is optionally substituted with one or more R¹²;
each R¹² is independently halogen, —OH, —CN, —NO₂, —OR^{a}, —NR^{c}R^{d}, C₁-C₆alkyl, C₁-C₆alkoxy, C₁-C₆haloalkyl, C₁-C₆hydroxyalkyl, C₁-C₆aminoalkyl, C₁-C₆heteroalkyl, C₃-C₆cycloalkyl, or 4 to 6 membered heterocycloalkyl;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
or R^{c} and R^{d} are taken together with the atom to which they are attached to form a heterocycloalkyl, wherein the heterocycloalkyl is optionally substituted with 1 to 4 substituents independently selected from R^{e};
each R^{e} is independently halogen, oxo, —CN, —OH, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, C₁-C₆heteroalkyl, or C₃-C₆cycloalkyl; and
p is 1, 2, 3, or 4.

In some embodiments, provided herein is a compound represented by Formula (I) or a pharmaceutically acceptable salt or a stereoisomer thereof. In some embodiments, provided herein is a compound represented by Formula (V) or a pharmaceutically acceptable salt or a stereoisomer thereof.

In some embodiments of Formula (I), the compound is not:

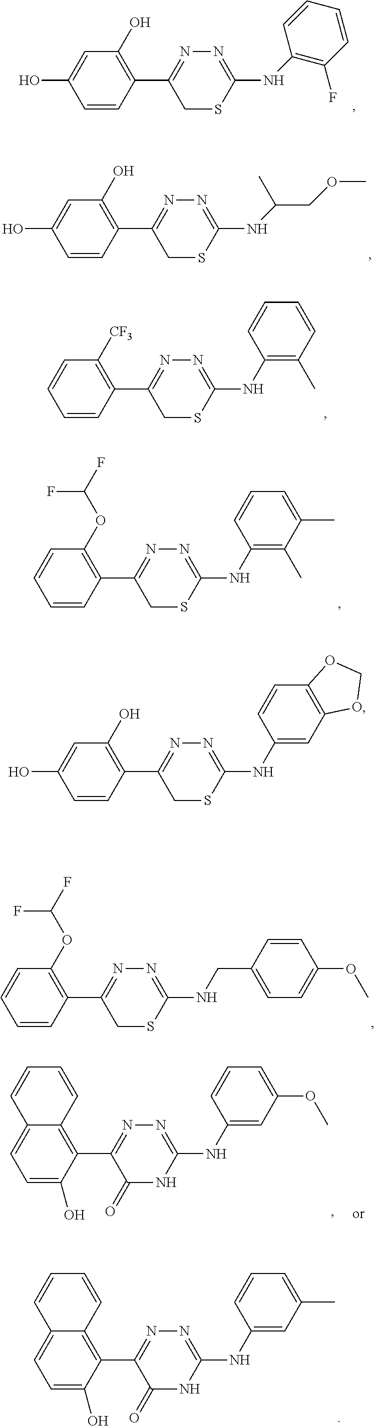

In some embodiments of Formula (I) or (V), R^{6A} is —OH. In some embodiments, R^{6A} is —CF₂H or —CF₃. In some embodiments, R^{6A} is —CF₂H. In some embodiments, R^{6A} is —CF₃, n some embodiments, R^{6A} is —OCF₂H.

In another aspect, the disclosure provides a compound represented by Formula (Ia) or Formula (Va), or a pharmaceutically acceptable salt or a stereoisomer thereof:

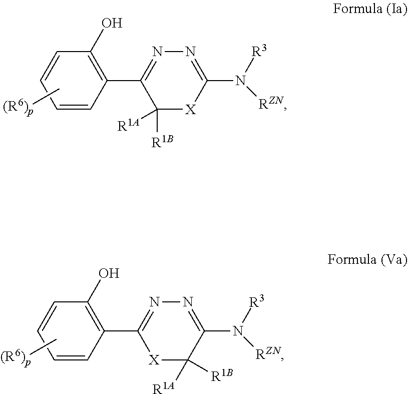

wherein R^{1A}, R^{1B}, R³, R^{ZN}, R⁶, X and p have the same meanings as described herein. In some embodiments, the R^{1A}, R^{1B}, R³, R^{ZN}, R⁶, X and p of Formula (Ia) have the meanings described in Formula (I). In some embodiments, the R^{1A}, R^{1B}, R³, R^{ZN}, R⁶, X and p of Formula (Va) have the meanings described in Formula (V).

In some embodiments, provided herein is a compound represented by Formula (Ia) or a pharmaceutically acceptable salt or a stereoisomer thereof. In some embodiments, provided herein is a compound represented by Formula (Va) or a pharmaceutically acceptable salt or a stereoisomer thereof.

In some embodiment of Formula (Ia), the compound is not:

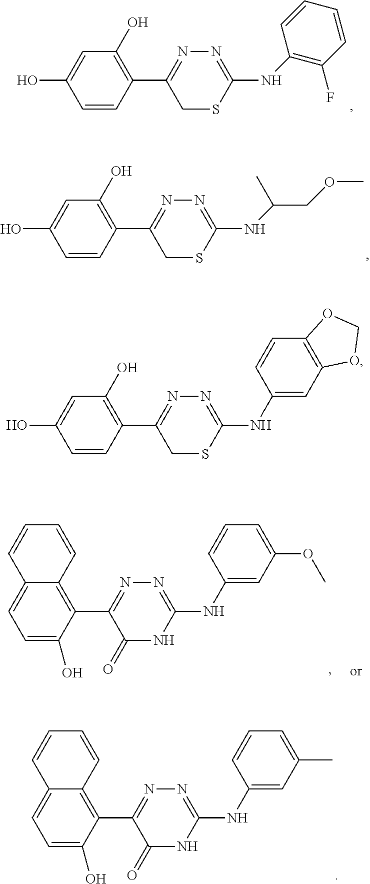 [SMILES: Oc1ccc(C2=NN=C(Nc3ccccc3F)SC2)c(O)c1 ; COCC(C)NC1=NN=C(c2ccc(O)cc2O)CS1 ; Oc1ccc(C2=NN=C(Nc3ccc4c(c3)OCO4)SC2)c(O)c1 ; COc1cccc(Nc2nnc(-c3c(O)ccc4ccccc34)c(=O)[nH]2)c1 ; Cc1cccc(Nc2nnc(-c3c(O)ccc4ccccc34)c(=O)[nH]2)c1]

In another aspect, the disclosure provides a compound represented by Formula (Ia) or Formula (Va), or a pharmaceutically acceptable salt or a stereoisomer thereof:

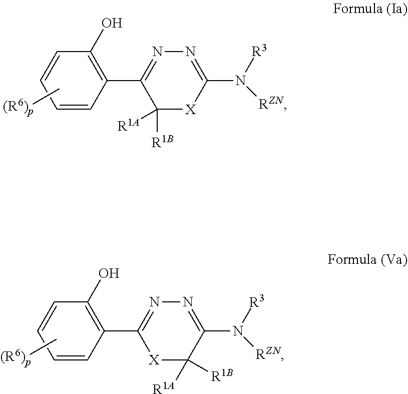

wherein;
X is —S—, —S(O)—, or —S(O)₂—;
R^{1A}, R^{1B}, R³, R^{ZN}, R⁶ and p have the same meanings as described herein. In some embodiments, the R^{1A},
R^{1B}, R³, R^{ZN}, R⁶ and p of Formula (Ia) have the meanings described in Formula (I). In some embodiments, the R^{1A}, R^{1B}, R³, R^{ZN}, R⁶ and p of Formula (Va) have the meanings described in Formula (V).

In some embodiment of Formula (Ia), the compound is not:

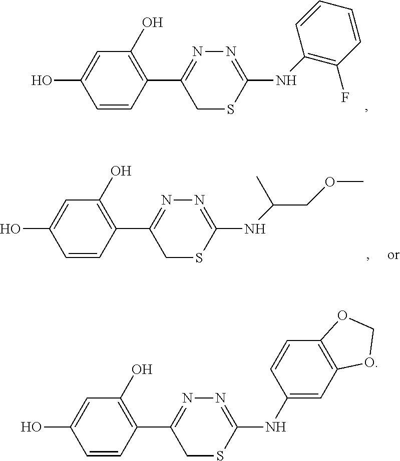 [SMILES: Oc1ccc(C2=NN=C(Nc3ccccc3F)SC2)c(O)c1 ; COCC(C)NC1=NN=C(c2ccc(O)cc2O)CS1 ; Oc1ccc(C2=NN=C(Nc3ccc4c(c3)OCO4)SC2)c(O)c1]

In some embodiments of Formula (I), (Ia), (V), or (Va) p is 2 or 3. In some embodiments, p is 2.

In some embodiments of Formula (Ia), the compound of Formula (Ia) has the structure of Formula (II), or a pharmaceutically acceptable salt or a stereoisomer thereof:

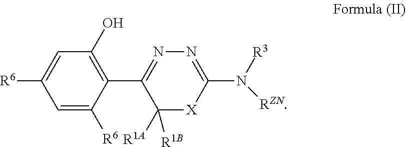

In another aspect, the disclosure provides a compound represented by Formula (III*), or a pharmaceutically acceptable salt or a stereoisomer thereof:

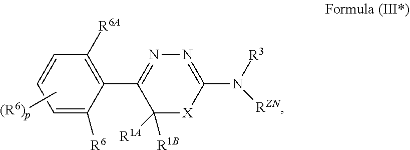

Wherein, p is 0, 1, 2, or 3;
R^{1A}, RIB, R³, R^{ZN}, R⁶, R^{6A} and X have the same meanings as described herein. In some embodiments, the R^{1A}, R^{1B}, R³, R^{ZN}, R⁶, R^{6A} and X of Formula (III*) have the meanings described in Formula (I). In some embodiments, the R^{1A}, R^{1B}, R³, R^{ZN}, R⁶, and X of Formula (III*) have the meanings described in Formula (III).

In some embodiments of Formula (III*), R^{6A} is —OH. In some embodiments, R^{6A} is —CF₂H or —CF₃. In some embodiments, R^{6A} is —CF₂H. In some embodiments, R^{6A} is —CF₃. In some embodiments, R^{6A} is —OCF₂H.

In another aspect, the disclosure provides a compound represented by Formula (III), or a pharmaceutically acceptable salt or a stereoisomer thereof:

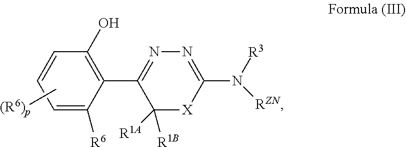

wherein;
X is —NR^{X}—, —O—, —S—, —S(O)—, or —S(O)₂—;
R^{X} is hydrogen, C₁-C₆alkyl, C₁-C₆haloalkyl, C₁-C₆heteroalkyl, C₁-C₆hydroxyalkyl, C₁-C₆aminoalkyl, C₃-C₆cycloalkyl, or 4 to 6 membered heterocycloalkyl; wherein each of the alkyl, haloalkyl, hydroxyalkyl, heteroalkyl, aminoalkyl, cycloalkyl, or heterocycloalkyl is optionally substituted with 1 to 4 substituents independently selected from R^{e};
each R^{1A} and R^{1B} is independently hydrogen, halogen, —CN, —NO₂, —OR^{a}, —NR^{c}R^{d}, C₁-C₆alkyl, C₁-C₆haloalkyl, C₁-C₆hydroxyalkyl, or C₁-C₆aminoalkyl;
or R^{1A} and R^{1B} are taken together to form an oxo;
or R^{1A} and R^{1B} are taken together to form a C₃-C₈cycloalkyl or 4 to 8 membered heterocycloalkyl; each of which is optionally substituted with one or more R¹¹;
each R¹¹ is independently halogen, —OH, —CN, —NO₂, —OR^{a}, —NR^{c}R^{d}, C₁-C₆alkyl, C₁-C₆alkoxy, C₁-C₆haloalkyl, C₁-C₆hydroxyalkyl, C₁-C₆aminoalkyl, C₁-C₆heteroalkyl, C₃-C₆cycloalkyl, or 4 to 6 membered heterocycloalkyl;
R³ is phenyl, 5 to 12 membered heteroaryl, C₃-C₁₂cycloalkyl, 4 to 12 membered heterocycloalkyl, or C₁-C₆alkyl; each of which is optionally substituted with one or more R⁸;
each R⁸ is independently halogen, —OH, —CN, —NO₂, —OR^{a}, —OC(═O)R^{a}, —OC(═O)OR^{b}, —OC(═O)NR^{c}R^{d}, —SH, —SR^{a}, SF₅, —S(═O)R^{a}, —S(═O)₂R^{a}, —S(═O)(═NR^{b})R^{a}, —S(═O)₂NR^{c}R⁴, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)R^{c}R^{d}, —P(═O)R^{c}R^{d}, —C(═O)R^{a}, —C(═O) OR′, —C(═O)NR^{c}R^{d}, C₁-C₆alkyl, C₁-C₆haloalkyl, C₁-C₆hydroxyalkyl, C₁-C₆heteroalkyl, C₁-C₆aminoalkyl, C₃-C₆cycloalkyl, or 4 to 6 membered heterocycloalkyl, wherein each of the alkyl, haloalkyl, hydroxyalkyl, heteroalkyl, aminoalkyl, cycloalkyl, or heterocycloalkyl is optionally substituted with 1 to 4 substituents independently selected from R^{e};
R^{ZN} is hydrogen, C₁-C₆alkyl, or C₁-C₆haloalkyl;
or R^{X} and R^{ZN} together with the atoms to which they are attached form a 4 to 8 membered ring which is optionally substituted with one or more R¹³;
each R¹³ is independently halogen, —OH, —CN, —NO₂, —OR^{a}, —NR^{c}R^{d}, C₁-C₆alkyl, C₁-C₆alkoxy, C₁-C₆haloalkyl, C₁-C₆hydroxyalkyl, or C₁-C₆aminoalkyl;
each R⁶ is independently halogen, —CN, —NO₂, —OH, —OR^{a}, —SH, —SR^{a}, —SF₅, —S(═O)R^{a}, —S(═O)₂R^{a}, —S(═O)₂NR^{c}R⁴, —NR^{c}R^{d}, —NR^{b}C(═O)NR^{c}R^{d}, —NR^{b}C(═O)R^{a}, —NR^{b}C(═O)OR^{b}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, or C₃-C₈cycloalkyl, wherein each of the alkyl, haloalkyl, hydroxyalkyl, heteroalkyl, aminoalkyl, alkenyl, alkynyl, or cycloalkyl is optionally substituted with 1 to 4 substituents independently selected from R^{e};
or two R^{e} are taken together with the atoms to which they are attached to form an aryl, heteroaryl, cycloalkyl or heterocycloalkyl, each of which is optionally substituted with one or more R¹²;
each R¹² is independently halogen, —OH, —CN, —NO₂, —OR^{a}, —NR^{c}R^{d}, C₁-C₆alkyl, C₁-C₆alkoxy, C₁-C₆haloalkyl, C₁-C₆hydroxyalkyl, C₁-C₆aminoalkyl, C₁-C₆heteroalkyl, C₃-C₆cycloalkyl, or 4 to 6 membered heterocycloalkyl;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each of the alkyl, haloalkyl, hydroxyalkyl, aminoalkyl, heteroalkyl, alkenyl, alkynyl, cycloalkyl, heterocycloalkyl, aryl or heteroaryl is optionally substituted with 1 to 4 substituents independently selected from R^{e};
or R^{c} and R^{d} are taken together with the atom to which they are attached to form a heterocycloalkyl, wherein the heterocycloalkyl is optionally substituted with 1 to 4 substituents independently selected from R^{e};
each R^{e} is independently halogen, oxo, —CN, —OH, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, C₁-C₆heteroalkyl, or C₃-C₆cycloalkyl; and
p is 0, 1, 2, or 3.

In another aspect, the disclosure provides a compound represented by Formula (IIIa), Formula (VIa), or a pharmaceutically acceptable salt or a stereoisomer thereof:

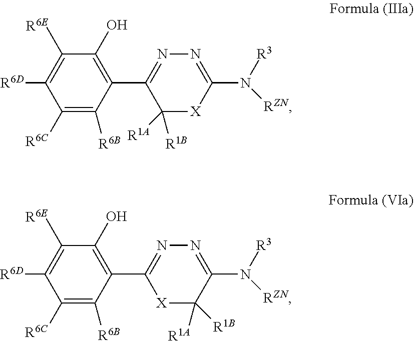

wherein, each R^{6B}, R^{6C}, R^{6D} and R^{6E} is independently hydrogen or R⁶;
R^{1A}, R^{1B}, R³, R^{ZN}, R⁶ and X have the same meanings as described herein. In some embodiments, the R^{1A}, R^{1B}, R³, R^{ZN}, R⁶ and X of Formula (IIIa) or (VIa) have the meanings described in Formula (III). In some embodiments, the R^{1A}, R^{1B}, R³, R^{ZN}, R⁶ and X of Formula (IIIa) or (VIa) have the meanings described in Formula (I). In some embodiments, R^{6B} is R⁶. In some embodiments, R^{6B} is H. In some embodiments, R^{6C} is R⁶. In some embodiments, R^{6C} is H. In some embodiments, R^{6D} is R⁶. In some embodiments, R^{6D} is H. In some embodiments, R^{6E} is R⁶. In some embodiments, R^{6E} is H. In some embodiments, R^{6B}, R^{6D}, R^{6E} are independently R⁶, and R^{6C} is H. In some embodiments, R^{6D} and R^{6E} are taken together with the atoms to which they are attached to form an aryl, heteroaryl, cycloalkyl or heterocycloalkyl, each of which is optionally substituted with one or more R¹². In some embodiments, R^{6D} and R^{6E} are taken together with the atoms to which they are attached to form an 5-6 membered heterocycloalkyl, each of which is optionally substituted with one or more R¹². In some embodiments, R^{6D} and R^{6E} are taken together with the atoms to which they are attached to form an 4-6 membered cycloalkyl, each of which is optionally substituted with one or more R¹².

In some embodiments, provided herein is a compound represented by Formula (IIIa) or a pharmaceutically acceptable salt or a stereoisomer thereof. In some embodiments, provided herein is a compound represented by Formula (VIa) or a pharmaceutically acceptable salt or a stereoisomer thereof.

In some embodiments of Formula (A), (I), (Ia), (II), (III*), (III), or (IIIa), R^{X} and R^{ZN} together with the atoms to which they are attached form a 5 to 8 membered heteroalkyl which is optionally substituted with one or more R¹³. In some embodiments, R^{X} and R^{ZN} together with the atoms to which they are attached form a 6 membered heterocycloalkyl. In some embodiments, R^{X} and R^{ZN} together with the atoms to which they are attached form a 7 membered heteroalkyl. In some embodiments, R^{X} and R^{ZN} together with the atoms to which they are attached form a 8 membered heteroalkyl. In some embodiments, R³ and R^{ZN} together with the atoms to which they are attached form a 5 to 13 membered heterocycloalkyl which is optionally substituted with one or more R¹³.

In another aspect, the disclosure provides a compound represented by Formula (IV), or a pharmaceutically acceptable salt or a stereoisomer thereof:

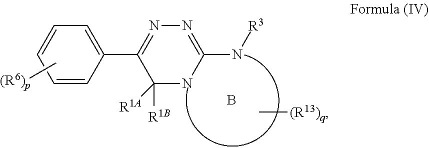

wherein, ring B is a 5 to 8-membered heterocycloalkyl;
each R¹³ is independently halogen, —OH, —CN, —NO₂, —OR^{a}, —NR^{c}R^{d}, C₁-C₆alkyl, C₁-C₆alkoxy, C₁-C₆haloalkyl, C₁-C₆hydroxyalkyl, or C₁-C₆aminoalkyl;
q is 0, 1, 2, or 3;
R^{1A}, R^{1B}, R³, R⁶ and p have the same meanings as described herein. In some embodiments, the R^{1A}, R^{1B}, R³, R⁶ and p of Formula (IV) have the meanings described in Formula (I). In some embodiments, the R^{1A}, R^{1B}, R³, R⁶ and p of Formula (IV) have the meanings described in Formula (A).

In some embodiments of Formula (IV), ring B is a 5 membered heterocycloalkyl. In some embodiments, ring B is a 6 membered heterocycloalkyl. In some embodiments, ring B is a 7 membered heterocycloalkyl. In some embodiments, ring B is a 8 membered heterocycloalkyl.

In some embodiments, the compound of Formula (IV) has the structure of Formula (IVa), or a pharmaceutically acceptable salt or a stereoisomer thereof:

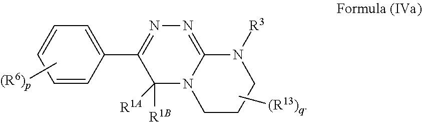

In some embodiments of Formula (A), (B), (I), (Ia), (II), (III*), (III), (IIIa), (V), (Va), or (VIa), X is —S—, —S(O)—, or —S(O)₂—. In some embodiments, X is —S—. In some embodiments, X is —S(O)—. In some embodiments, X is —S(O)₂—. In some embodiments, X is —O—. In some embodiments, X is NR^{X}.

In some embodiments of Formula (A), (B), (I), (Ia), (II), (III*), (III), (IIIa), (V), (Va), or (VIa), R^{ZN} is hydrogen, C₁-C₆alkyl, or C₁-C₆haloalkyl. In some embodiments, R^{ZN} is hydrogen or C₁-C₆alkyl. In some embodiments, R^{ZN} is C₁-C₆alkyl. In some embodiments, R^{ZN} is methyl. In some embodiments, R^{ZN} is hydrogen.

In some embodiments of Formula (A), (B), (I), (Ia), (II), (III*), (III), (IIIa), (V), (Va), or (VIa), R^{X} is hydrogen, C₁-C₆alkyl, C₁-C₆haloalkyl, C₁-C₆heteroalkyl, C₁-C₆hydroxyalkyl, C₁-C₆aminoalkyl, C₃-C₆cycloalkyl, or 4 to 6 membered heterocycloalkyl; wherein each of the alkyl, haloalkyl, hydroxyalkyl, heteroalkyl, aminoalkyl, cycloalkyl, or heterocycloalkyl is optionally substituted with 1 to 4 substituents independently selected from R^{e}. In some embodiments, R^{X} is hydrogen or C₁-C₆alkyl. In some embodiments, R^{X} is C₁-C₆alkyl. In some embodiments, R^{X} is methyl or ethyl. In some embodiments, R^{X} is methyl. In some embodiments, R^{X} is ethyl. In some embodiments, R^{X} is hydrogen.

In some embodiments of Formula (A), (B), (I), (Ia), (II), (III*), (III), (IIIa), (IV), (IVa), (V), (Va), or (VIa), R^{1A} and R^{1B} are each independently hydrogen, halogen, —CN, —NO₂, —OH, —OR^{a}, —NR^{c}R^{d}, C₁-C₆alkyl, C₁-C₆haloalkyl, C₁-C₆hydroxyalkyl, or C₁-C₆aminoalkyl. In some embodiments, R^{1A} and R^{1B} are each independently halogen, C₁-C₆alkyl, or C₁-C₆haloalkyl. In some embodiments, R^{1A} and R^{1B} are each independently halogen or C₁-C₆haloalkyl. In some embodiments, R^{1A} and R^{1B} are each independently fluoro, chloro, bromo, CF³, or CHF². In some embodiments, R^{1A} and R^{1B} are each CF₃. In some embodiments, R^{1A} and R^{1B} are each independently C₁-C₆alkyl. In some embodiments, R^{1A} and R^{1B} are each independently methyl, ethyl, n-propyl, isopropyl, n-butyl, sec-butyl, or tert-butyl. In some embodiments, R^{1A} and R^{1B} are each independently methyl. In some embodiments, R^{1A} and R^{1B} are each independently hydrogen.

In some embodiments of Formula (A), (B), (I), (Ia), (II), (III*), (III), (IIIa), (IV), (IVa), (V), (Va), or (VIa), R^{1A} is hydrogen, halogen, —CN, —NO₂, —OH, —OR^{a}, —NR^{c}R^{d}, C₁-C₆alkyl, C₁-C₆haloalkyl, C₁-C₆hydroxyalkyl, or C₁-C₆aminoalkyl. In some embodiments, R^{1A} is halogen, C₁-C₆alkyl, or C₁-C₆haloalkyl. In some embodiments, R^{1A} is halogen or C₁-C₆haloalkyl. In some embodiments, R^{1A} is fluoro, chloro, bromo, CF³, or CHF². In some embodiments, R^{1A} is CF₃. In some embodiments, R^{1A} is C₁-C₆alkyl. In some embodiments, R^{1A} is methyl, ethyl, n-propyl, isopropyl, n-butyl, sec-butyl, or tert-butyl. In some embodiments, R^{1A} is methyl. In some embodiments, R^{1A} is hydrogen.

In some embodiments of Formula (A), (B), (I), (Ia), (II), (III*), (III), (IIIa), (IV), (IVa), (V), (Va), or (VIa), R^{1B} is hydrogen, halogen, —CN, —NO₂, —OH, —OR^{a}, —NR^{c}R^{d}, C₁-C₆alkyl, C₁-C₆haloalkyl, C₁-C₆hydroxyalkyl, or C₁-C₆aminoalkyl. In some embodiments, R^{1B} is halogen, C₁-C₆alkyl, or C₁-C₆haloalkyl. In some embodiments, R^{1B} is halogen or C₁-C₆haloalkyl. In some embodiments, R^{1B} is fluoro, chloro, bromo, CF³, or CHF². In some embodiments, R^{1B} is CF₃. In some embodiments, R^{1B} is C₁-C₆alkyl. In some embodiments, R^{1B} is methyl, ethyl, n-propyl, isopropyl, n-butyl, sec-butyl, or tert-butyl. In some embodiments, R^{1B} is methyl. In some embodiments, R^{1B} is hydrogen.

In some embodiments of Formula (A), (B), (I), (Ia), (II), (III*), (III), (IIIa), (IV), (IVa), (V), (Va), or (VIa), R^{1A} and R^{1B} are taken together to form a C₃-C₈cycloalkyl or 4 to 8 membered heterocycloalkyl; each of which is optionally substituted with one or more R¹¹. In some embodiments, R^{1A} and R^{1B} are taken together to form a C₃-C₈cycloalkyl. In some embodiments, R^{1A} and R^{1B} are taken together to form a 4 to 8 membered heterocycloalkyl. In some embodiments, R¹ is cyclopropyl, cyclobutyl, cyclopentyl, or cyclohexyl. In some embodiments, R¹ is cyclopropyl.

In some embodiments of Formula (A), (B), (I), (Ia), (II), (III*), (III), (IIIa), (IV), (IVa), (V), (Va), or (VIa), R^{1A} and R^{1B} are taken together to form an oxo.

In some embodiments of Formula (A), (B), (I), (Ia), (II), (III*), (III), (IIIa), (IV), (IVa), (V), (Va), or (VIa), each R⁶ is independently halogen, —CN, —OH, —OR^{a}, —SH, —SR^{a}, —SF₅, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, or C₂-C₆alkynyl, wherein each of the alkyl, haloalkyl, hydroxyalkyl, heteroalkyl, aminoalkyl, alkenyl or alkynyl is optionally substituted with 1 to 4 substituents independently selected from R^{e}. In some embodiments, each R⁶ is independently halogen, —CN, —NO₂, —OH, —OR^{a}, —SH, —SR^{a}, —SF₅, —NR^{c}R^{d}, C₁-C₆alkyl, C₁-C₆haloalkyl, C₁-C₆hydroxyalkyl, or C₁-C₆aminoalkyl. In some embodiments, each R⁶ is independently halogen, —OH, C₁-C₆alkyl, C₁-C₆haloalkyl, or C₁-C₆hydroxyalkyl. In some embodiments, each R⁶ is independently halogen, C₁-C₆alkyl, C₁-C₆haloalkyl, or C₁-C₆hydroxyalkyl. In some embodiments, each R⁶ is independently halogen. In some embodiments, each R⁶ is independently fluoro or chloro. In some embodiments, each R⁶ is independently C₁-C₆alkyl. In some embodiments, each R⁶ is independently methyl, ethyl, n-propyl, isopropyl, n-butyl, sec-butyl, or tert-butyl. In some embodiments, R⁶ is methyl or ethyl. In some embodiments, R⁶ is methyl. In some embodiments, R⁶ is-OH. In some embodiments, each R⁶ is independently halogen, —OH, C₁-C₆alkyl, C₁-C₆alkoxyl, C₁-C₆haloalkyl, or C₁-C₆hydroxyalkyl, wherein the C₁-C₆alkoxyl is optionally substituted with 1-6 halogen. In some embodiments, each R⁶ is independently halogen, C₁-C₆alkyl, C₁-C₆haloalkyl, C₁-C₆hydroxyalkyl, or C₁-C₆alkoxyl, wherein the alkoxyl is optionally substituted with one or three halogen. In some embodiments, one or more R⁶ is independently halogen, —OH, C₁-C₆alkyl, C₁-C₆alkoxyl, C₁-C₆haloalkyl, or C₁-C₆hydroxyalkyl, wherein the C₁-C₆alkoxyl is optionally substituted with 1-6 halogen. In some embodiments, one or more R⁶ is independently halogen, —OH, C₁-C₆alkyl, C₁-C₆alkoxyl, C₁-C₆haloalkyl, or C₁-C₆hydroxyalkyl, wherein the C₁-C₆alkoxyl is optionally substituted with 1-6 halogen, and two R⁶ are taken together with the atoms to which they are attached to form an aryl, heteroaryl, cycloalkyl or heterocycloalkyl, each of which is optionally substituted with one or more R¹².

In some embodiments of Formula (A), (B), (I), (Ia), (II), (III*), (III), (IIIa), (IV), (IVa), (V), (Va), or (VIa), each R⁶ is independently C₁-C₆haloalkyl. In some embodiments, each R⁶ is independently CF₃, CF₂H, or CFH₂. In some embodiments, R⁶ is CF₃. In some embodiments, R⁶ is CF₂H. In some embodiments, R⁶ is CFH₂. In some embodiments, R⁶ is —OCF₃. In some embodiments, R⁶ is —OCHF₂. In some embodiments, R⁶ is —OCH₃.

In some embodiments of Formula (A), (B), (I), (Ia), (II), (III*), (III), (IIIa), (IV), (IVa), (V), (Va), or (VIa), two R⁶ are taken together with the atoms to which they are attached to form an aryl, heteroaryl, cycloalkyl or heterocycloalkyl, each of which is optionally substituted with one or more R¹². In some embodiments, two R⁶ are taken together with the atoms to which they are attached to form a heteroaryl. In some embodiments, two R⁶ are taken together with the atoms to which they are attached to form a cycloalkyl or heterocycloalkyl. In some embodiments, two R⁶ are taken together with the atoms to which they are attached to form a C₄-C₈ cycloalkyl. In some embodiments, two R⁶ are taken together with the atoms to which they are attached to form a C₅-C₆ cycloalkyl. In some embodiments, two R⁶ are taken together with the atoms to which they are attached to form a 4 to 8-membered heterocycloalkyl. In some embodiments, two R⁶ are taken together with the atoms to which they are attached to form a 5 to 6-membered heterocycloalkyl. In some embodiments, two R⁶ are taken together with the atoms to which they are attached to form an aryl, heteroaryl, cycloalkyl, or heterocycloalkyl, each of which is optionally substituted with one or more R¹². In some embodiments, two R⁶ are taken together with the atoms to which they are attached to form a cycloalkyl, or heterocycloalkyl, each of which is optionally substituted with one or more R¹². In some embodiments,

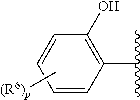

is

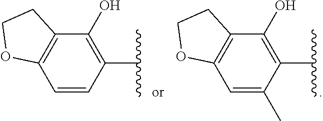

In some embodiments,

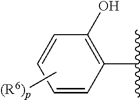

is

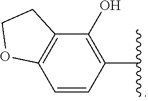 [SMILES: *c1ccc2c(c1O)CCO2]

In some embodiments,

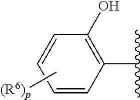

is

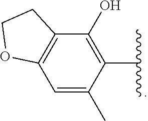 [SMILES: *c1c(C)cc2c(c1O)CCO2]

In some embodiments of Formula (A), (B), (I), (Ia), (II), (III*), (III), (IIIa), (IV), (IVa), (V), (Va), or (VIa), R³ is a C₃-C₁₂cycloalkyl or 4 to 12 membered heterocycloalkyl; each of which is optionally substituted with one or more R⁸. In some embodiments, R³ is a C₃-C₆cycloalkyl, which is optionally substituted with 1, 2, or 3 R⁸. In some embodiments, R³ is a 4 to 6 membered heterocycloalkyl, which is optionally substituted with 1, 2, or 3 R⁸. In some embodiments, R³ is

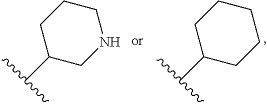 [SMILES: *C1CCCNC1 ; "or" ; *C1CCCCC1 ; ","]

each of which is optionally substituted with 1, 2, or 3 R⁸. In some embodiments, R⁸ is each independently selected from —OH, —OR^{a}, —SH, —SR^{a}, SF₅, —NR^{c}R^{d}, C₁-C₆alkyl, C₁-C₆haloalkyl, C₁-C₆hydroxyalkyl, C₁-C₆heteroalkyl, C₁-C₆aminoalkyl, and C₃-C₆cycloalkyl.

In some embodiments, R³ is attached to a compound of Formula (A), (B), (I), (Ia), (II), (III*), (III), (IIIa), (IV), (IVa), (V), (Va), or (VIa) via a chiral carbon atom of the R³ group. In some embodiments, the chiral carbon atom of the R³ group has an S configuration. In some embodiments, the chiral carbon atom of the R³ group has an R configuration.

In some embodiments of Formula (A), (B), (I), (Ia), (II), (III*), (III), (IIIa), (IV), (IVa), (V), (Va), or (VIa), R³ is

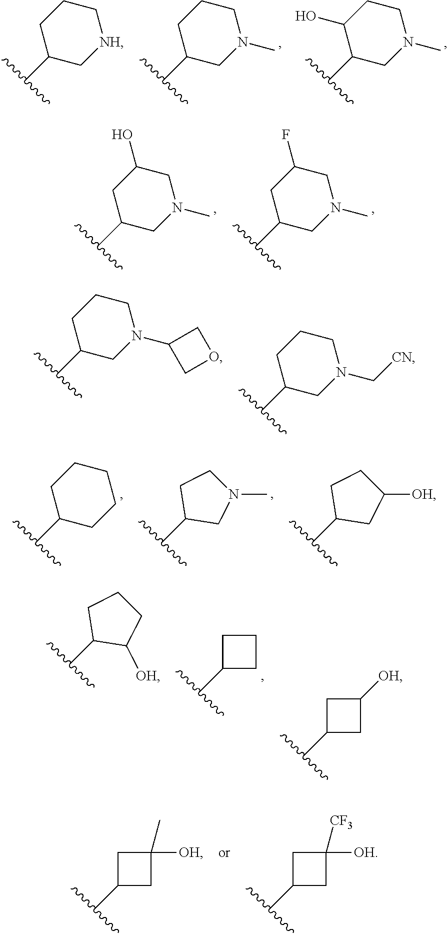

In some embodiments, R³ is

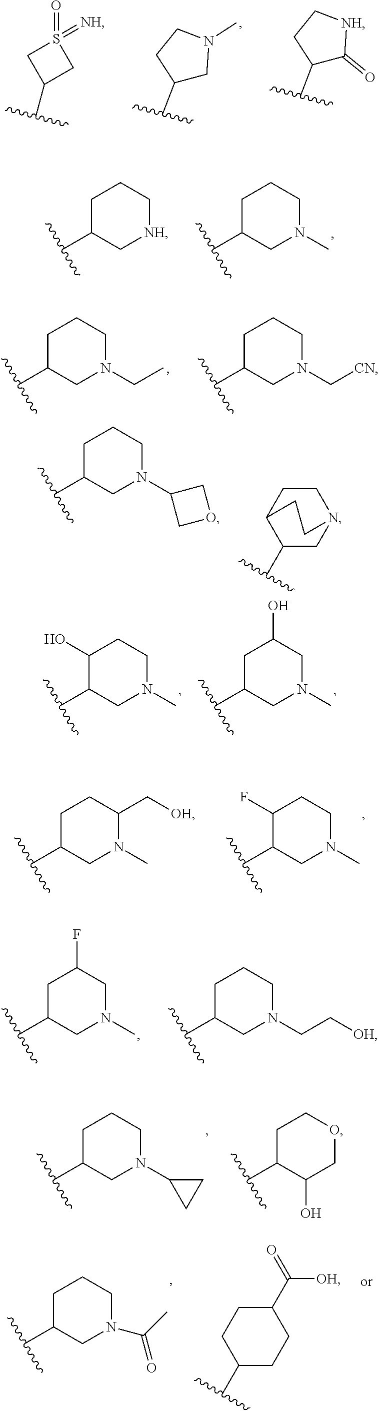

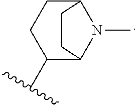 [SMILES: *C1CCC2CCC1N2C]

In some embodiments, R³ is

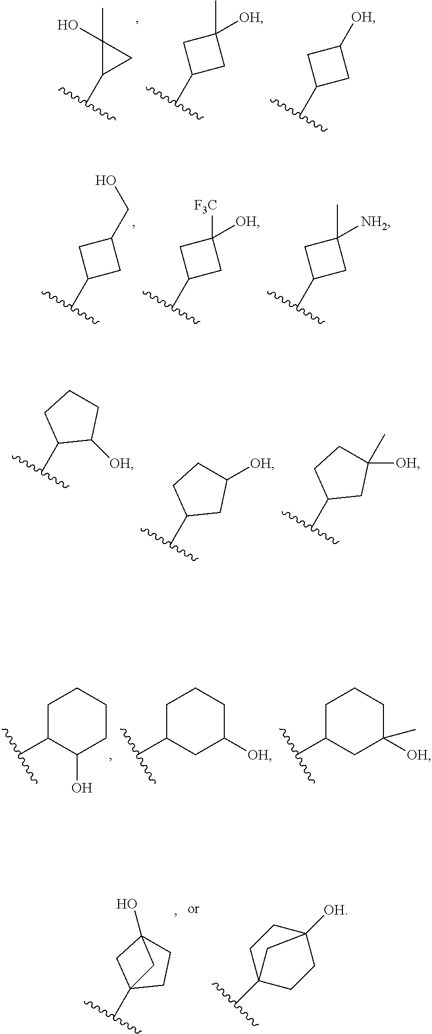

In some embodiments, R³ is

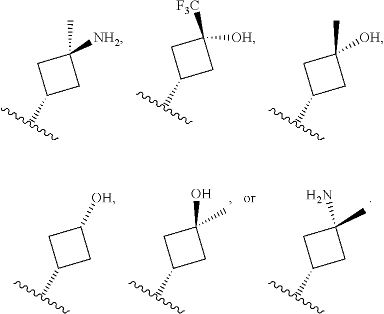

In some embodiments, R³ is

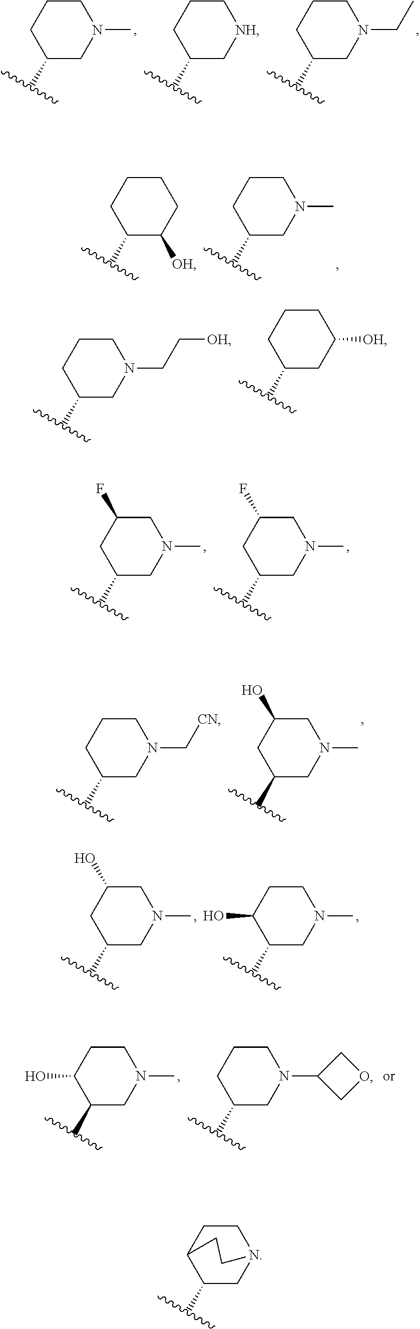

In some embodiments, R³ is

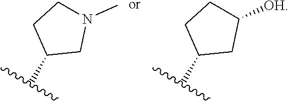

In some embodiments of Formula (A), (B), (I), (Ia), (II), (III*), (III), (IIIa), (IV), (IVa), (V), (Va), or (VIa), R³ is hydrogen, C₁-C₆alkyl, C₁-C₆haloalkyl, C₁-C₆hydroxyalkyl, C₁-C₆aminoalkyl, C₁-C₆heteroalkyl, C₃-C₁₂cycloalkyl, or 4 to 12 membered heterocycloalkyl; each of which is optionally substituted. In some embodiments, R³ is optionally substituted with one or more R⁸. In some embodiments, R³ is hydrogen. In some embodiments, R³ is optionally substituted C₁-C₆alkyl. In some embodiments, R³ is optionally substituted C₁-C₆haloalkyl. In some embodiments, R³ is optionally substituted C₁-C₆hydroxyalkyl. In some embodiments, R³ is

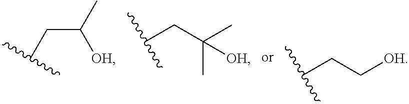

In some embodiments, R³ is

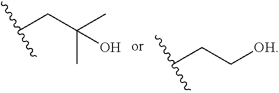

In some embodiments, R³ is

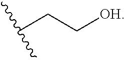 [SMILES: *CCO]

In some embodiments, R³ is

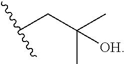 [SMILES: *CC(C)(C)O]

In some embodiments of Formula (A), (B), (I), (Ia), (II), (III*), (III), (IIIa), (IV), (IVa), (V), (Va), or (VIa), R³ is optionally substituted C₁-C₆aminoalkyl. In some embodiments, R³ is optionally substituted C₁-C₆heteroalkyl. In some embodiments, R³ is optionally substituted C₃-C₁₂cycloalkyl. In some embodiments, R³ is optionally substituted 4 to 12 membered heterocycloalkyl.

In some embodiments of Formula (A), (B), (I), (Ia), (II), (III*), (III), (IIIa), (IV), (IVa), (V), (Va), or (VIa), R³ is a C₃-C₁₂cycloalkyl which is optionally substituted with one or more R⁸. In some embodiments, R³ is a C₄-C₆cycloalkyl which is optionally substituted with one or more R⁸. In some embodiments, R³ is cyclobutyl, which is optionally substituted with one or more R⁸. In some embodiments, R³ is cyclopentyl, which is optionally substituted with one or more R⁸. In some embodiments, R³ is cyclohexyl, which is optionally substituted with one or more R⁸. In some embodiments, R³ is a cyclopropyl, cyclobutyl, cyclopentyl or cyclohexyl. In some embodiments, R³ is monocyclic. In some embodiments, R³ is bicyclic.

In some embodiments of Formula (A), (B), (I), (Ia), (II), (III*), (III), (IIIa), (IV), (IVa), (V), (Va), or (VIa), R³ is a 4 to 12 membered heterocycloalkyl which is optionally substituted with one or more R⁸. In some embodiments, R³ is a 4 to 8-membered heterocycloalkyl. In some embodiments, R³ is a 5 to 6-membered heterocycloalkyl. In some embodiments, R³ is a 6-membered heterocycloalkyl. In some embodiments, R³ is a 5-membered heterocycloalkyl. In some embodiments, R³ is a 4-membered heterocycloalkyl. In some embodiments, R³ is monocyclic. In some embodiments, R³ is bicyclic.

In some embodiments of Formula (A), (B), (I), (Ia), (II), (III*), (III), (IIIa), (IV), (IVa), (V), (Va), or (VIa), R³ is

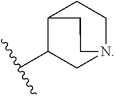 [SMILES: *C1CN2CCC1CC2]

In some embodiments of Formula (A), (B), (I), (Ia), (II), (III*), (III), (IIIa), (IV), (IVa), (V), (Va), or (VIa), R³ is cyclohexyl or piperidine. In some embodiments, R³ is piperidine. In some embodiments, R³ is morpholine. In some embodiments, R³ is cyclohexyl. In some embodiments, R³ is cyclopentyl. In some embodiments, R³ is cyclobutyl.

In some embodiments of Formula (A), (B), (I), (Ia), (II), (III*), (III), (IIIa), (IV), (IVa), (V), (Va), or (VIa), R³ is phenyl or 5 to 12 membered heteroaryl each of which is optionally substituted with one or more R⁸. In some embodiments, R³ is phenyl. In some embodiments, R³ is 5 to 12 membered heteroaryl. In some embodiments, R³ is 5 to 10 membered heteroaryl. In some embodiments, R³ is 5 to 6 membered heteroaryl. In some embodiments, R³ is 5 membered heteroaryl. In some embodiments, R³ is 6 membered heteroaryl.

In some embodiments of Formula (A), (B), (I), (Ia), (II), (III*), (III), (IIIa), (IV), (IVa), (V), (Va), or (VIa), R³ is optionally substituted with one to three R⁸.

In some embodiments of Formula (A), (B), (I), (Ia), (II), (III*), (III), (IIIa), (IV), (IVa), (V), (Va), or (VIa), R³ is

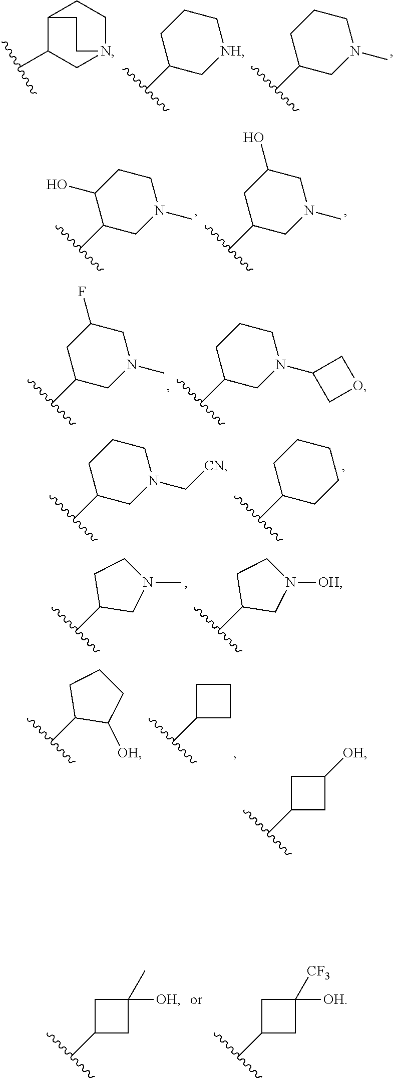

In some embodiments, R³ is

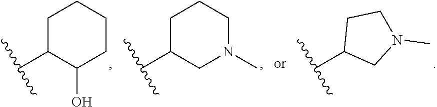

In some embodiments, R³ is

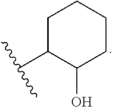 [SMILES: *C1CCCCC1O]

In some embodiments, R³ is

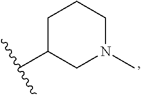 [SMILES: *C1CCCN(C)C1]

In some embodiments, R³ is

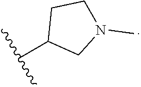 [SMILES: *C1CCN(C)C1]

In some embodiments of Formula (A), (B), (I), (Ia), (II), (III*), (III), (IIIa), (IV), (IVa), (V), (Va), or (VIa), each R⁸ is independently halogen, —OH, —CN, —NO₂, —OR^{a}, —OC(═O)R^{a}, —OC(═O)OR^{b}, —OC(═O)NR^{c}R^{d}, —SH, —SR^{a}, SF₅, —S(═O)R^{a}, —S(═O)₂R^{a}, —S(═O)(═NR^{b})R^{a}, —S(═O)₂NR^{c}R⁴, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)R^{c}R^{d}, —P(═O)R^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆heteroalkyl, C₁-C₆aminoalkyl, C₃-C₆cycloalkyl, or 4 to 6 membered heterocycloalkyl, wherein each of the alkyl, haloalkyl, hydroxyalkyl, heteroalkyl, aminoalkyl, cycloalkyl, or heterocycloalkyl is optionally substituted with 1 to 4 substituents independently selected from R^{e}. In some embodiments, each R⁸ is independently halogen, —OH, C₁-C₆alkyl, C₃-C₆cycloalkyl, or 4 to 6 membered heterocycloalkyl, wherein each of the alkyl, haloalkyl, hydroxyalkyl, heteroalkyl, aminoalkyl, cycloalkyl, or heterocycloalkyl is optionally substituted with 1 to 4 substituents independently selected from R^{e}.

In some embodiments of Formula (A), (B), (I), (Ia), (II), (III*), (III), (IIIa), (IV), (IVa), (V), (Va), or (VIa), each R⁸ is independently halogen. In some embodiments, each R⁸ is independently fluoro, bromo or chloro. In some embodiments, each R⁸ is independently —OH. In some embodiments, each R⁸ is independently C₁-C₆alkyl. In some embodiments, each R⁸ is independently methyl, ethyl, n-propyl, isopropyl, n-butyl, sec-butyl, or tert-butyl.

In some embodiments of Formula (A), (B), (I), (Ia), (II), (III*), (III), (IIIa), (IV), (IVa), (V), (Va), or (VIa), each R⁸ is independently C₃-C₆cycloalkyl or 4 to 6 membered heterocycloalkyl. In some embodiments, each R⁸ is independently C₃-C₆cycloalkyl. In some embodiments, each R⁸ is independently cyclopropyl, cyclobutyl, or cyclopentyl. In some embodiments, each R⁸ is independently 4 to 6 membered heterocycloalkyl. In some embodiments, each R⁸ is independently 4-membered heterocycloalkyl. In some embodiments, each R⁸ is independently 5-membered heterocycloalkyl. In some embodiments, R⁸ is each independently selected from —OH, —OR^{a}, —SH, —SR^{a}, SF₅, —NR^{c}R^{d}, C₁-C₆alkyl, C₁-C₆haloalkyl, C₁-C₆hydroxyalkyl, C₁-C₆heteroalkyl, C₁-C₆aminoalkyl, and C₃-C₆cycloalkyl.

In some embodiments of Formula (A), (B), (I), (Ia), (II), (III*), (III), (IIIa), (IV), (IVa), (V), (Va), or (VIa), each R¹¹ is independently halogen, —OH, —NR^{c}R^{d}, C₁-C₆alkyl, or C₁-C₆haloalkyl. In some embodiments, each R¹¹ is independently halogen, or —OH, —NH₂. In some embodiments, each R¹¹ is independently C₁-C₆alkyl. In some embodiments, each R¹¹ is independently C₁-C₆haloalkyl.

In some embodiments of Formula (A), (B), (I), (Ia), (II), (III*), (III), (IIIa), (IV), (IVa), (V), (Va), or (VIa), each R¹² is independently halogen, —OH, —CN, —NO₂, —OR^{a}, —NR^{c}R^{d}, C₁-C₆alkyl, C₁-C₆alkoxy, C₁-C₆haloalkyl, C₁-C₆hydroxyalkyl, C₁-C₆aminoalkyl, C₁-C₆heteroalkyl, C₃-C₆cycloalkyl, or 4 to 6 membered heterocycloalkyl. In some embodiments, each R¹² is independently halogen, —OH, —CN, —NO₂, —OR^{a}, —NR^{c}R^{d}, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, C₁-C₆heteroalkyl, C₃-C₆cycloalkyl, or 4 to 6 membered heterocycloalkyl. In some embodiments, each R¹² is independently halogen, —OH, —CN, —NO₂, —OR^{a}, —NR^{c}R^{d}, C₁-C₆alkyl, C₁-C₆alkoxy, C₁-C₆haloalkyl, or C₁-C₆hydroxyalkyl, C₁-C₆aminoalkyl.

In some embodiments of Formula (A), (B), (I), (Ia), (II), (III*), (III), (IIIa), (IV), (IVa), (V), (Va), or (VIa), each R¹³ is independently halogen, —OH, —CN, —NO₂, —OR^{a}, —NR^{c}R^{d}, C₁-C₆alkyl, C₁-C₆alkoxy, C₁-C₆haloalkyl, C₁-C₆hydroxyalkyl, or C₁-C₆aminoalkyl. In some embodiments, each R¹³ is independently C₁-C₆alkyl. In some embodiments, each R¹³ is independently methyl, ethyl, n-propyl, isopropyl, sec-butyl, or tert-butyl.

In some embodiments of Formula (IV) or (IVa), p is 5.

In some embodiments of Formula (I), (Ia), (IV), (V), or (Va), p is 4.

In some embodiments of Formula (A), (B), (I), (Ia), (II), (III*), (III), (IV), (IVa), (V), or (Va), p is 3. In some embodiments, p is at least 3. In some embodiments, p is 1, 2, or 3. In some embodiments, p is 1 or 2. In some embodiments, p is 2. In some embodiments, p is 1.

In some embodiments of a compound disclosed herein, each R^{a} is independently C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene (cycloalkyl), C₁-C₆alkylene (heterocycloalkyl), C₁-C₆alkylene (aryl), or C₁-C₆alkylene (heteroaryl); wherein each alkyl, cycloalkyl, heterocycloalkyl, aryl, and heteroaryl is independently optionally substituted with one or more R. In some embodiments of a compound disclosed herein, each R^{a} is independently C₁-C₆alkyl, C₁-C₆haloalkyl, or cycloalkyl, heterocycloalkyl; wherein each alkyl, cycloalkyl, and heterocycloalkyl is independently optionally substituted with one or more R. In some embodiments of a compound disclosed herein, each R^{a} is independently C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene (cycloalkyl), C₁-C₆alkylene (heterocycloalkyl), C₁-C₆alkylene (aryl), or C₁-C₆alkylene (heteroaryl). In some embodiments of a compound disclosed herein, each R^{a} is independently C₁-C₆alkyl, C₁-C₆haloalkyl, or cycloalkyl, heterocycloalkyl. In some embodiments of a compound disclosed herein, each R^{a} is independently C₁-C₆alkyl or C₁-C₆haloalkyl. In some embodiments of a compound disclosed herein, each R^{a} is independently C₁-C₆alkyl.

In some embodiments of a compound disclosed herein, each R^{b} is independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene (cycloalkyl), C₁-C₆alkylene (heterocycloalkyl), C₁-C₆alkylene (aryl), or C₁-C₆alkylene (heteroaryl); wherein each alkyl, cycloalkyl, heterocycloalkyl, aryl, and heteroaryl is independently optionally substituted with one or more R. In some embodiments of a compound disclosed herein, each R^{b} is independently hydrogen, C₁-C₆alkyl, C₁-C₆haloalkyl, or cycloalkyl, heterocycloalkyl; wherein each alkyl, cycloalkyl, and heterocycloalkyl is independently optionally substituted with one or more R. In some embodiments of a compound disclosed herein, each R^{b} is independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene (cycloalkyl), C₁-C₆alkylene (heterocycloalkyl), C₁-C₆alkylene (aryl), or C₁-C₆alkylene (heteroaryl). In some embodiments of a compound disclosed herein, each R^{b} is independently hydrogen, C₁-C₆alkyl, C₁-C₆haloalkyl, or cycloalkyl, heterocycloalkyl. In some embodiments of a compound disclosed herein, each R^{b} is independently hydrogen, C₁-C₆alkyl or C₁-C₆haloalkyl. In some embodiments of a compound disclosed herein, each R^{b} is independently hydrogen or C₁-C₆alkyl. In some embodiments of a compound disclosed herein, each R^{b} is hydrogen. In some embodiments of a compound disclosed herein, each R^{b} is independently C₁-C₆alkyl.

In some embodiments of a compound disclosed herein, each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene (cycloalkyl), C₁-C₆alkylene (heterocycloalkyl), C₁-C₆alkylene (aryl), or C₁-C₆alkylene (heteroaryl); wherein each alkyl, cycloalkyl, heterocycloalkyl, aryl, and heteroaryl is independently optionally substituted with one or more R. In some embodiments of a compound disclosed herein, each R^{c} and R^{d} are independently hydrogen, C₁-C₆alkyl, C₁-C₆haloalkyl, or cycloalkyl, heterocycloalkyl; wherein each alkyl, cycloalkyl, and heterocycloalkyl is independently optionally substituted with one or more R. In some embodiments of a compound disclosed herein, each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene (cycloalkyl), C₁-C₆alkylene (heterocycloalkyl), C₁-C₆alkylene (aryl), or C₁-C₆alkylene (heteroaryl). In some embodiments of a compound disclosed herein, each R^{c} and R^{d} are independently hydrogen, C₁-C₆alkyl, C₁-C₆haloalkyl, or cycloalkyl, heterocycloalkyl. In some embodiments of a compound disclosed herein, each R^{c} and R^{d} are independently hydrogen, C₁-C₆alkyl or C₁-C₆haloalkyl. In some embodiments of a compound disclosed herein, each R^{c} and R^{d} are independently hydrogen or C₁-C₆alkyl. In some embodiments of a compound disclosed herein, each R^{c} and R^{d} are hydrogen. In some embodiments of a compound disclosed herein, each R^{c} and R^{d} are independently C₁-C₆alkyl.

In some embodiments of a compound disclosed herein, R^{c} and R^{d} are taken together with the atom to which they are attached to form a heterocycloalkyl optionally substituted with one or more R^{e}.

In some embodiments of a compound disclosed herein, each R^{e} is independently halogen, oxo, —CN, —OH, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, C₁-C₆heteroalkyl, or C₃-C₆cycloalkyl. In some embodiments of a compound disclosed herein, each R^{e} is independently halogen, oxo, —CN, —OH, —S(═O)C₁₋₆alkyl, —S(═O)₂C₁₋₆alkyl, —S(═O)₂NH₂, —S(═O)₂NHC₁₋₆alkyl, —S(═O)₂N(C₁₋₆alkyl)₂, —NH₂, —NHC₁₋₆alkyl, —N(C₁₋₆alkyl)₂, —C(═O)C₁₋₆alkyl, —C(═O)OH, —C(═O)OC₁₋₆alkyl, —NHC(═O)C₁₋₆alkyl, C₁-C₆alkyl, C₁-C₆alkoxy, C₁-C₆haloalkyl, C₁-C₆hydroxyalkyl, C₁-C₆aminoalkyl, C₁-C₆heteroalkyl, or C₃-C₆cycloalkyl. In some embodiments, R^{e} is —NHC(═O)C₁₋₃alkyl such as —NHC(═O)CH₃. In some embodiments of a compound disclosed herein, each R^{e} is independently halogen, —CN, —OH, or C₁-C₆alkyl. In some embodiments of a compound disclosed herein, each R^{e} is independently halogen, —OH, or C₁-C₆alkyl. In some embodiments of a compound disclosed herein, each R^{e} is independently halogen or C₁-C₆alkyl. In some embodiments of a compound disclosed herein, each R^{e} is independently halogen.

In some embodiments of a compound disclosed herein, one or more of R^{1A}, R^{1B}, R³, R⁶, R⁸, R¹¹, R¹², R¹³, R^{ZN}, R^{X}, R^{a}, R^{b}, R^{c}, and R^{d}, and R^{e} groups comprise deuterium at a percentage higher than the natural abundance of deuterium.

In some embodiments of a compound disclosed herein, one or more 1H are replaced with one or more deuteriums in one or more of the following groups R^{1A}, R^{1B}, R³, R⁶, R⁸, R¹¹, R¹², R¹³, R^{ZN}, R^{X}, R^{a}, R^{b}, R^{c}, and R^{d}, and R^{e}.

In some embodiments of a compound disclosed herein, the abundance of deuterium in each of R^{1A}, R^{1B}, R³, R⁶, R⁸, R¹¹, R¹², R¹³, R^{ZN}, R^{X}, R^{a}, R^{b}, R^{c}, and R^{d}, and R^{e} is independently at least 1%, at least 10%, at least 20%, at least 30%, at least 40%, at least 50%, at least 60%, at least 70%, at least 80%, at least 90%, or 100% by molar.

Any combination of the groups described above for the various variables is contemplated herein. Throughout the specification, groups and substituents thereof are chosen by one skilled in the field to provide stable moieties and compounds.

In some embodiments the compound disclosed herein, or a pharmaceutically acceptable salt or a stereoisomer thereof, is one of the compounds in Table 1 or Table 2.

TABLE 1

| Cpd. | |
|---|---|
| No. | Structure |
| 1 | 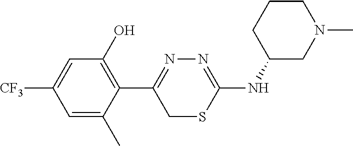 [SMILES: Cc1cc(C(F)(F)F)cc(O)c1C1=NN=C(N[C@@H]2CCCN(C)C2)SC1] |
| 2 | 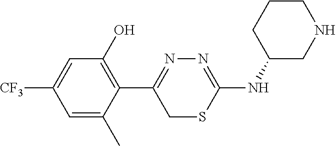 [SMILES: Cc1cc(C(F)(F)F)cc(O)c1C1=NN=C(N[C@@H]2CCCNC2)SC1] |
| 3 | 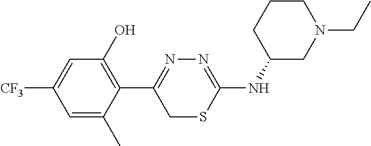 [SMILES: CCN1CCC[C@@H](NC2=NN=C(c3c(C)cc(C(F)(F)F)cc3O)CS2)C1] |
| 4 | 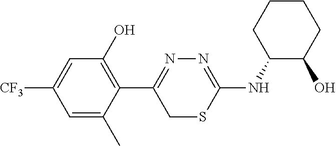 [SMILES: Cc1cc(C(F)(F)F)cc(O)c1C1=NN=C(N[C@@H]2CCCC[C@H]2O)SC1] |
| 5 | 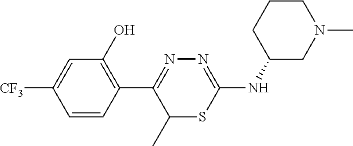 [SMILES: CC1SC(N[C@@H]2CCCN(C)C2)=NN=C1c1ccc(C(F)(F)F)cc1O] |
| 6 | 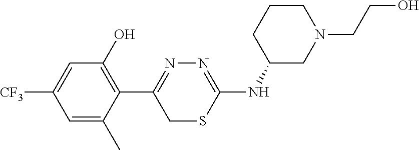 [SMILES: Cc1cc(C(F)(F)F)cc(O)c1C1=NN=C(N[C@@H]2CCCN(CCO)C2)SC1] |
| 7 | 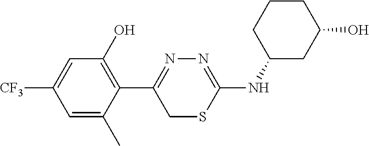 [SMILES: Cc1cc(C(F)(F)F)cc(O)c1C1=NN=C(N[C@@H]2CCC[C@H](O)C2)SC1] |
| 8 | 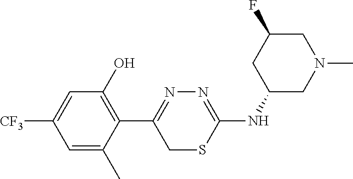 [SMILES: Cc1cc(C(F)(F)F)cc(O)c1C1=NN=C(N[C@@H]2C[C@@H](F)CN(C)C2)SC1] |
| 9 | 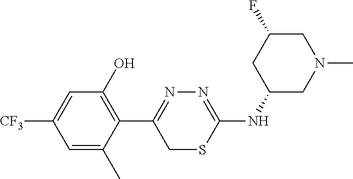 [SMILES: Cc1cc(C(F)(F)F)cc(O)c1C1=NN=C(N[C@@H]2C[C@H](F)CN(C)C2)SC1] |
| 10 | 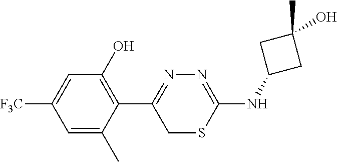 [SMILES: Cc1cc(C(F)(F)F)cc(O)c1C1=NN=C(N[C@H]2C[C@@](C)(O)C2)SC1] |
| 11 | 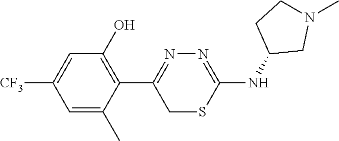 [SMILES: Cc1cc(C(F)(F)F)cc(O)c1C1=NN=C(N[C@@H]2CCN(C)C2)SC1] |
| 12 | 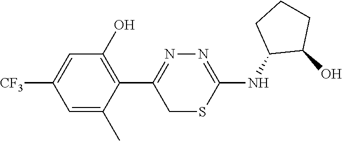 [SMILES: Cc1cc(C(F)(F)F)cc(O)c1C1=NN=C(N[C@@H]2CCC[C@H]2O)SC1] |
| 13 | 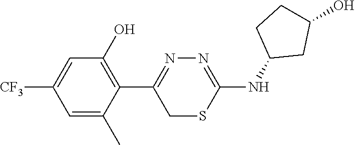 [SMILES: Cc1cc(C(F)(F)F)cc(O)c1C1=NN=C(N[C@@H]2CC[C@H](O)C2)SC1] |
| 14 | 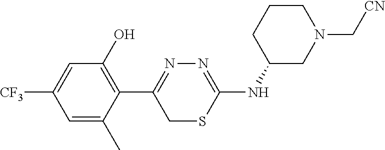 [SMILES: Cc1cc(C(F)(F)F)cc(O)c1C1=NN=C(N[C@@H]2CCCN(CC#N)C2)SC1] |
| 15 and 16 | 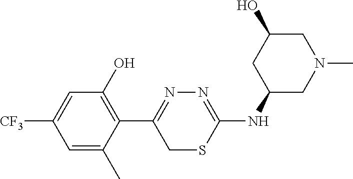 [SMILES: Cc1cc(C(F)(F)F)cc(O)c1C1=NN=C(N[C@H]2C[C@@H](O)CN(C)C2)SC1] |
| | 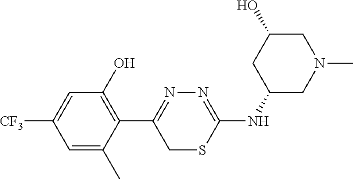 [SMILES: Cc1cc(C(F)(F)F)cc(O)c1C1=NN=C(N[C@@H]2C[C@H](O)CN(C)C2)SC1] |
| 17 | 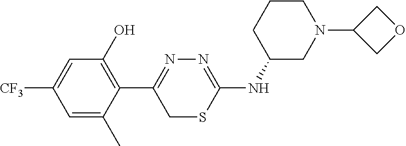 [SMILES: Cc1cc(C(F)(F)F)cc(O)c1C1=NN=C(N[C@@H]2CCCN(C3COC3)C2)SC1] |
| 18 and 19 | 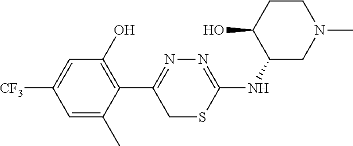 [SMILES: Cc1cc(C(F)(F)F)cc(O)c1C1=NN=C(N[C@H]2CN(C)CC[C@@H]2O)SC1] |
| | 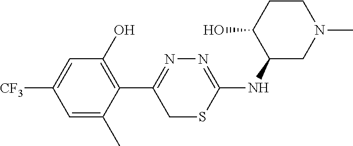 [SMILES: Cc1cc(C(F)(F)F)cc(O)c1C1=NN=C(N[C@@H]2CN(C)CC[C@H]2O)SC1] |
| 20 | 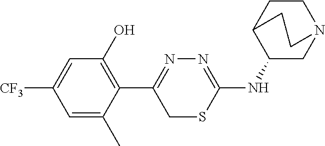 [SMILES: Cc1cc(C(F)(F)F)cc(O)c1C1=NN=C(N[C@H]2CN3CCC2CC3)SC1] |
| 21 | 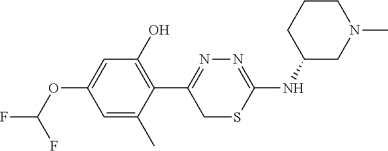 [SMILES: Cc1cc(OC(F)F)cc(O)c1C1=NN=C(N[C@@H]2CCCN(C)C2)SC1] |
| 22 | 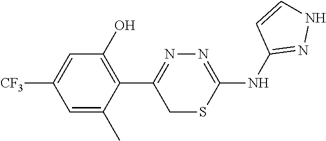 [SMILES: Cc1cc(C(F)(F)F)cc(O)c1C1=NN=C(Nc2cc[nH]n2)SC1] |
| 23 | 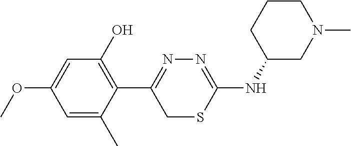 |
| 24 | 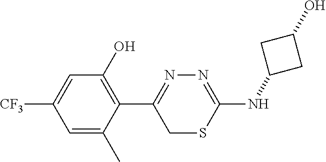 |
| 25 | 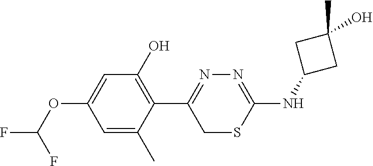 [SMILES: Cc1cc(OC(F)F)cc(O)c1C1=NN=C(N[C@H]2C[C@@](C)(O)C2)SC1] |
| 26 | 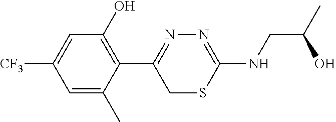 [SMILES: Cc1cc(C(F)(F)F)cc(O)c1C1=NN=C(NC[C@@H](C)O)SC1] |
| 27 | 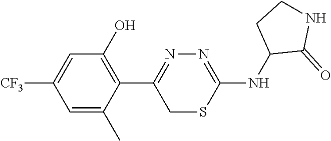 [SMILES: Cc1cc(C(F)(F)F)cc(O)c1C1=NN=C(NC2CCNC2=O)SC1] |
| 28 | 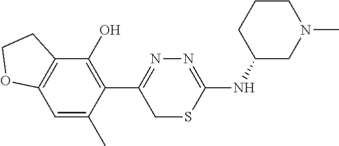 [SMILES: Cc1cc2c(c(O)c1C1=NN=C(N[C@@H]3CCCN(C)C3)SC1)CCO2] |
| 29 | 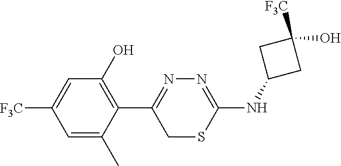 [SMILES: Cc1cc(C(F)(F)F)cc(O)c1C1=NN=C(N[C@H]2C[C@](O)(C(F)(F)F)C2)SC1] |
| 30 | 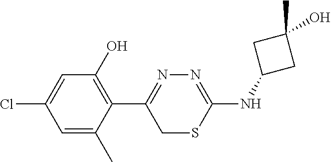 [SMILES: Cc1cc(Cl)cc(O)c1C1=NN=C(N[C@H]2C[C@@](C)(O)C2)SC1] |
| 31 | 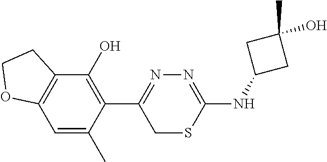 [SMILES: Cc1cc2c(c(O)c1C1=NN=C(N[C@H]3C[C@@](C)(O)C3)SC1)CCO2] |
| 32 | 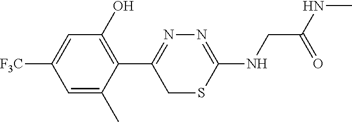 [SMILES: CNC(=O)CNC1=NN=C(c2c(C)cc(C(F)(F)F)cc2O)CS1] |
| 33 and 34 | 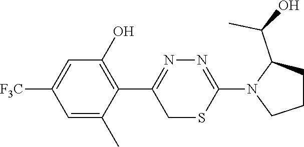 [SMILES: Cc1cc(C(F)(F)F)cc(O)c1C1=NN=C(N2CCC[C@@H]2[C@@H](C)O)SC1] |
| | 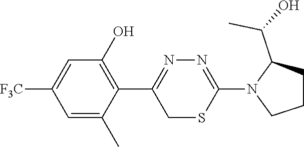 [SMILES: Cc1cc(C(F)(F)F)cc(O)c1C1=NN=C(N2CCC[C@@H]2[C@H](C)O)SC1] |
| 35 | 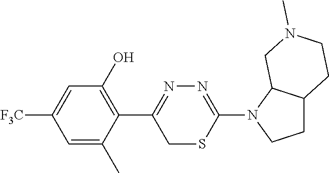 [SMILES: Cc1cc(C(F)(F)F)cc(O)c1C1=NN=C(N2CCC3CCN(C)CC32)SC1] |
| 36 | 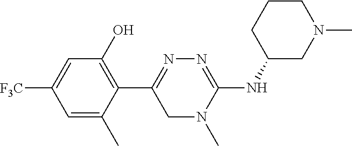 [SMILES: Cc1cc(C(F)(F)F)cc(O)c1C1=NN=C(N[C@@H]2CCCN(C)C2)N(C)C1] |
| 37 | 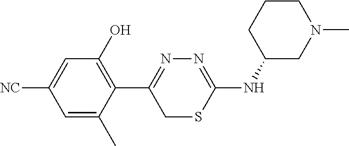 [SMILES: Cc1cc(C#N)cc(O)c1C1=NN=C(N[C@@H]2CCCN(C)C2)SC1] |
| 38 | 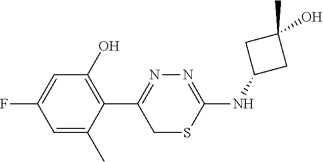 [SMILES: Cc1cc(F)cc(O)c1C1=NN=C(N[C@H]2C[C@@](C)(O)C2)SC1] |
| 39 | 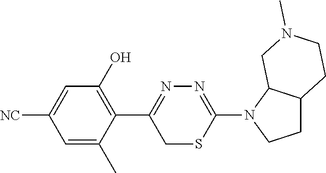 [SMILES: Cc1cc(C#N)cc(O)c1C1=NN=C(N2CCC3CCN(C)CC32)SC1] |
| 40 | 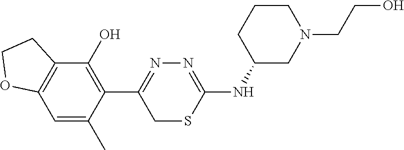 [SMILES: Cc1cc2c(c(O)c1C1=NN=C(N[C@@H]3CCCN(CCO)C3)SC1)CCO2] |
| 41 | 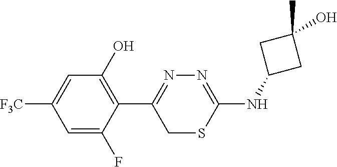 [SMILES: C[C@]1(O)C[C@@H](NC2=NN=C(c3c(O)cc(C(F)(F)F)cc3F)CS2)C1] |
| 42 | 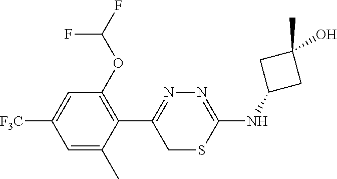 [SMILES: Cc1cc(C(F)(F)F)cc(OC(F)F)c1C1=NN=C(N[C@H]2C[C@@](C)(O)C2)SC1] |
| 43 | 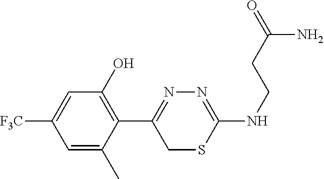 [SMILES: Cc1cc(C(F)(F)F)cc(O)c1C1=NN=C(NCCC(N)=O)SC1] |
| 44 | 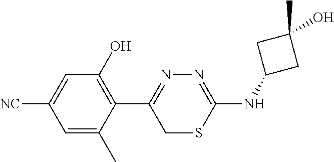 [SMILES: Cc1cc(C#N)cc(O)c1C1=NN=C(N[C@H]2C[C@@](C)(O)C2)SC1] |
| 45 | 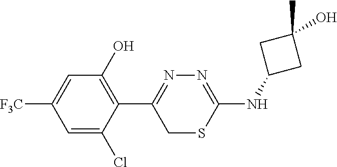 [SMILES: C[C@]1(O)C[C@@H](NC2=NN=C(c3c(O)cc(C(F)(F)F)cc3Cl)CS2)C1] |
| 46 | 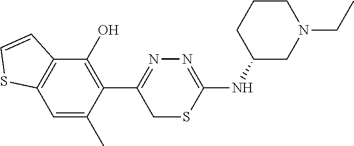 [SMILES: CCN1CCC[C@@H](NC2=NN=C(c3c(C)cc4sccc4c3O)CS2)C1] |
| 47 | 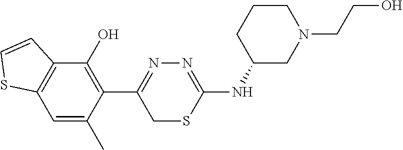 [SMILES: Cc1cc2sccc2c(O)c1C1=NN=C(N[C@@H]2CCCN(CCO)C2)SC1] |
| 48 | 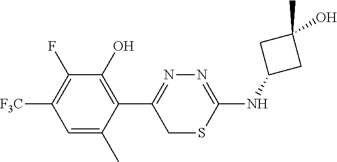 [SMILES: Cc1cc(C(F)(F)F)c(F)c(O)c1C1=NN=C(N[C@H]2C[C@@](C)(O)C2)SC1] |
| 49 | 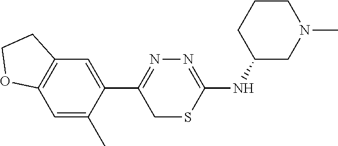 [SMILES: Cc1cc2c(cc1C1=NN=C(N[C@@H]3CCCN(C)C3)SC1)CCO2] |
| 50 | 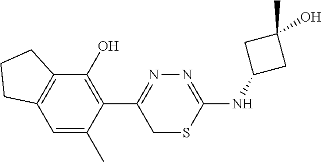 [SMILES: Cc1cc2c(c(O)c1C1=NN=C(N[C@H]3C[C@@](C)(O)C3)SC1)CCC2] |
| 51 | 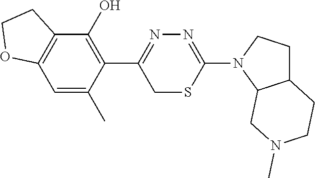 [SMILES: Cc1cc2c(c(O)c1C1=NN=C(N3CCC4CCN(C)CC43)SC1)CCO2] |
| 52 | 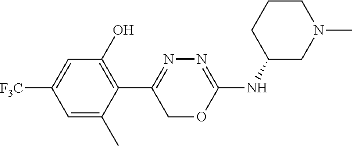 [SMILES: Cc1cc(C(F)(F)F)cc(O)c1C1=NN=C(N[C@@H]2CCCN(C)C2)OC1] |
| 53 | 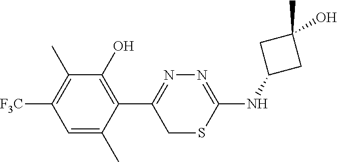 [SMILES: Cc1cc(C(F)(F)F)c(C)c(O)c1C1=NN=C(N[C@H]2C[C@@](C)(O)C2)SC1] |
| 54 | 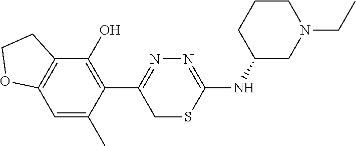 [SMILES: CCN1CCC[C@@H](NC2=NN=C(c3c(C)cc4c(c3O)CCO4)CS2)C1] |
| 55 | 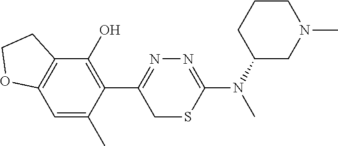 [SMILES: Cc1cc2c(c(O)c1C1=NN=C(N(C)[C@@H]3CCCN(C)C3)SC1)CCO2] |
| 56 | 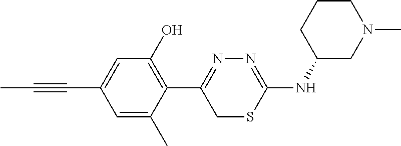 [SMILES: CC#Cc1cc(C)c(C2=NN=C(N[C@@H]3CCCN(C)C3)SC2)c(O)c1] |
| 57 | 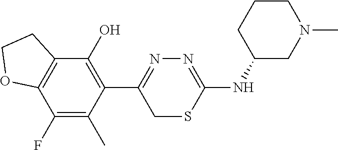 [SMILES: Cc1c(F)c2c(c(O)c1C1=NN=C(N[C@@H]3CCCN(C)C3)SC1)CCO2] |
| 58 | 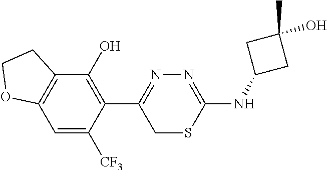 [SMILES: C[C@]1(O)C[C@@H](NC2=NN=C(c3c(C(F)(F)F)cc4c(c3O)CCO4)CS2)C1] |
| 59 | 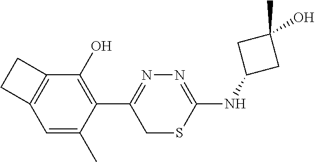 [SMILES: Cc1cc2c(c(O)c1C1=NN=C(N[C@H]3C[C@@](C)(O)C3)SC1)CC2] |
| 60 | 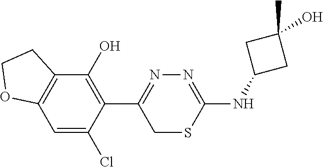 [SMILES: C[C@]1(O)C[C@@H](NC2=NN=C(c3c(Cl)cc4c(c3O)CCO4)CS2)C1] |
| 61 | 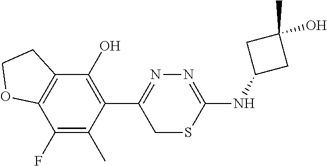 [SMILES: Cc1c(F)c2c(c(O)c1C1=NN=C(N[C@H]3C[C@@](C)(O)C3)SC1)CCO2] |
| 62 | 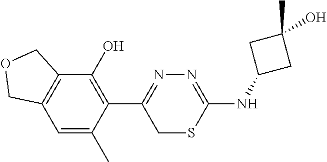 [SMILES: Cc1cc2c(c(O)c1C1=NN=C(N[C@H]3C[C@@](C)(O)C3)SC1)COC2] |
| 63 | 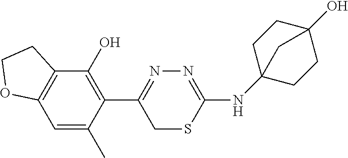 [SMILES: Cc1cc2c(c(O)c1C1=NN=C(NC34CCC(O)(CC3)C4)SC1)CCO2] |
| 64 | 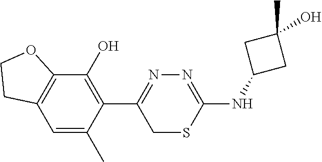 [SMILES: Cc1cc2c(c(O)c1C1=NN=C(N[C@H]3C[C@@](C)(O)C3)SC1)OCC2] |
| 65 | 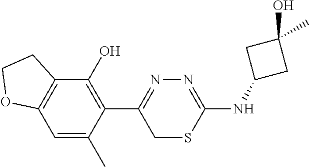 [SMILES: Cc1cc2c(c(O)c1C1=NN=C(N[C@H]3C[C@](C)(O)C3)SC1)CCO2] |
| 66 | 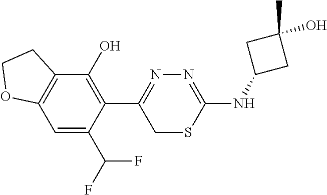 [SMILES: C[C@]1(O)C[C@@H](NC2=NN=C(c3c(C(F)F)cc4c(c3O)CCO4)CS2)C1] |
| 67 | 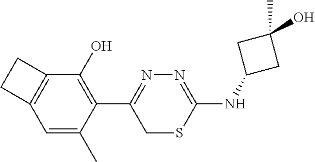 [SMILES: Cc1cc2c(c(O)c1C1=NN=C(N[C@H]3C[C@](C)(O)C3)SC1)CC2] |
| 68 | 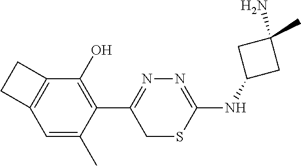 [SMILES: Cc1cc2c(c(O)c1C1=NN=C(N[C@H]3C[C@@](C)(N)C3)SC1)CC2] |
| 69 | 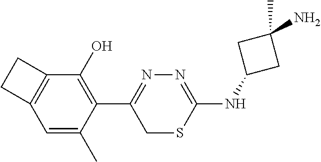 [SMILES: Cc1cc2c(c(O)c1C1=NN=C(N[C@H]3C[C@](C)(N)C3)SC1)CC2] |
| 70 | 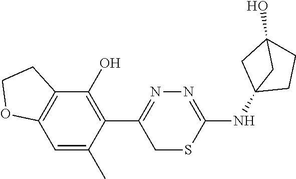 [SMILES: Cc1cc2c(c(O)c1C1=NN=C(N[C@]34CC[C@](O)(C3)C4)SC1)CCO2] |
| 71 | 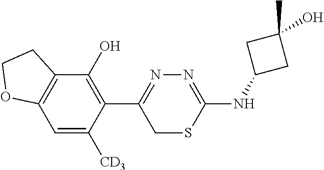 [SMILES: [2H]C([2H])([2H])c1cc2c(c(O)c1C1=NN=C(N[C@H]3C[C@@](C)(O)C3)SC1)CCO2] |
| 201 | 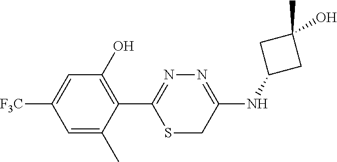 [SMILES: Cc1cc(C(F)(F)F)cc(O)c1C1=NN=C(N[C@H]2C[C@@](C)(O)C2)CS1] |
| 202 | 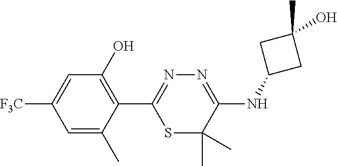 [SMILES: Cc1cc(C(F)(F)F)cc(O)c1C1=NN=C(N[C@H]2C[C@@](C)(O)C2)C(C)(C)S1] |
| 203 | 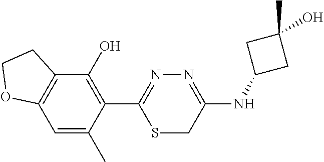 [SMILES: Cc1cc2c(c(O)c1C1=NN=C(N[C@H]3C[C@@](C)(O)C3)CS1)CCO2] |
| 204 | 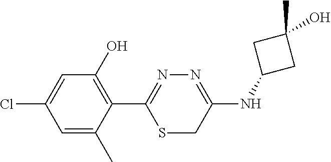 [SMILES: Cc1cc(Cl)cc(O)c1C1=NN=C(N[C@H]2C[C@@](C)(O)C2)CS1] |
| 205 | 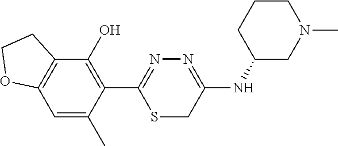 [SMILES: Cc1cc2c(c(O)c1C1=NN=C(N[C@@H]3CCCN(C)C3)CS1)CCO2] |
| 206 | 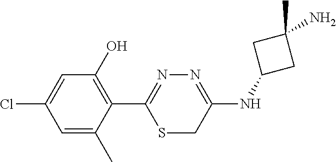 [SMILES: Cc1cc(Cl)cc(O)c1C1=NN=C(N[C@H]2C[C@@](C)(N)C2)CS1] |
| 207 | 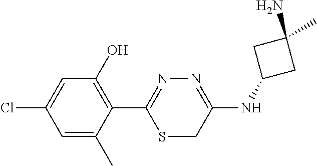 [SMILES: Cc1cc(Cl)cc(O)c1C1=NN=C(N[C@H]2C[C@](C)(N)C2)CS1] |
| 72 | 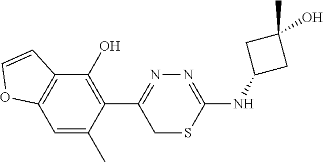 [SMILES: Cc1cc2occc2c(O)c1C1=NN=C(N[C@H]2C[C@@](C)(O)C2)SC1] |

TABLE 2

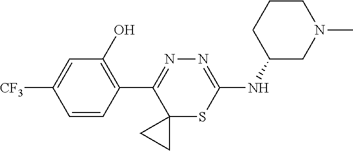 [SMILES: CN1CCC[C@@H](NC2=NN=C(c3ccc(C(F)(F)F)cc3O)C3(CC3)S2)C1]

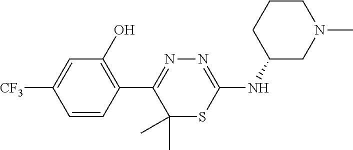 [SMILES: CN1CCC[C@@H](NC2=NN=C(c3ccc(C(F)(F)F)cc3O)C(C)(C)S2)C1]

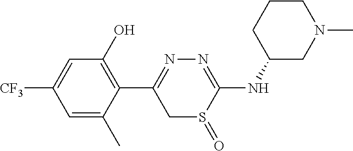 [SMILES: Cc1cc(C(F)(F)F)cc(O)c1C1=NN=C(N[C@@H]2CCCN(C)C2)S(=O)C1]

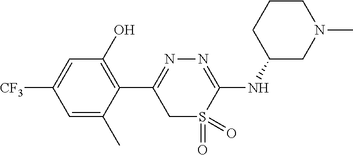 [SMILES: Cc1cc(C(F)(F)F)cc(O)c1C1=NN=C(N[C@@H]2CCCN(C)C2)S(=O)(=O)C1]

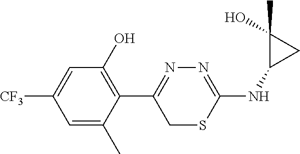 [SMILES: Cc1cc(C(F)(F)F)cc(O)c1C1=NN=C(N[C@H]2C[C@@]2(C)O)SC1]

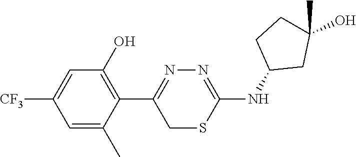 [SMILES: Cc1cc(C(F)(F)F)cc(O)c1C1=NN=C(N[C@@H]2CC[C@](C)(O)C2)SC1]

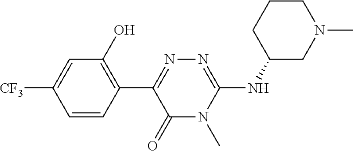 [SMILES: CN1CCC[C@@H](Nc2nnc(-c3ccc(C(F)(F)F)cc3O)c(=O)n2C)C1]

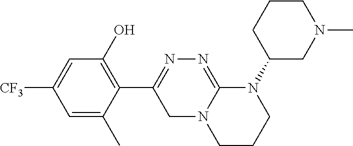 [SMILES: Cc1cc(C(F)(F)F)cc(O)c1C1=NN=C2N(CCCN2[C@@H]2CCCN(C)C2)C1]

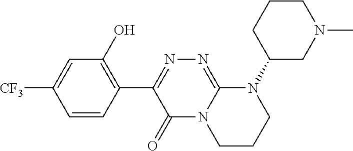 [SMILES: CN1CCC[C@@H](N2CCCn3c2nnc(-c2ccc(C(F)(F)F)cc2O)c3=O)C1]

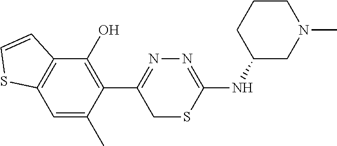 [SMILES: Cc1cc2sccc2c(O)c1C1=NN=C(N[C@@H]2CCCN(C)C2)SC1]

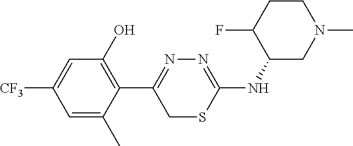 [SMILES: Cc1cc(C(F)(F)F)cc(O)c1C1=NN=C(N[C@H]2CN(C)CCC2F)SC1]

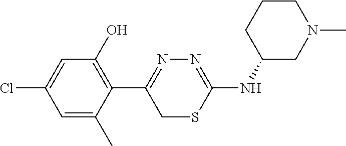 [SMILES: Cc1cc(Cl)cc(O)c1C1=NN=C(N[C@@H]2CCCN(C)C2)SC1]

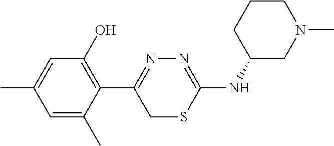

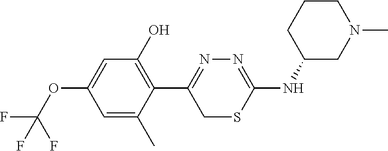 [SMILES: Cc1cc(OC(F)(F)F)cc(O)c1C1=NN=C(N[C@@H]2CCCN(C)C2)SC1]

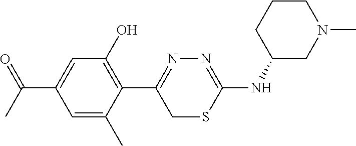 [SMILES: CC(=O)c1cc(C)c(C2=NN=C(N[C@@H]3CCCN(C)C3)SC2)c(O)c1]

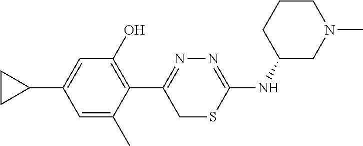 [SMILES: Cc1cc(C2CC2)cc(O)c1C1=NN=C(N[C@@H]2CCCN(C)C2)SC1]

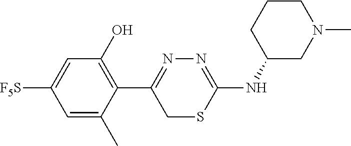

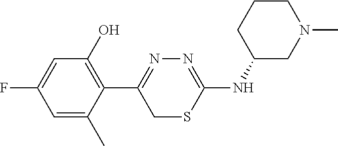 [SMILES: Cc1cc(F)cc(O)c1C1=NN=C(N[C@@H]2CCCN(C)C2)SC1]

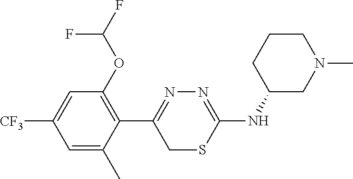 [SMILES: Cc1cc(C(F)(F)F)cc(OC(F)F)c1C1=NN=C(N[C@@H]2CCCN(C)C2)SC1]

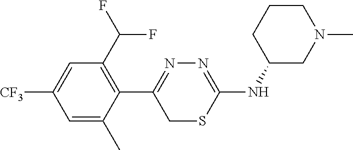 [SMILES: Cc1cc(C(F)(F)F)cc(C(F)F)c1C1=NN=C(N[C@@H]2CCCN(C)C2)SC1]

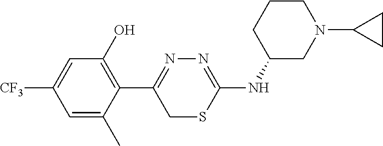 [SMILES: Cc1cc(C(F)(F)F)cc(O)c1C1=NN=C(N[C@@H]2CCCN(C3CC3)C2)SC1]

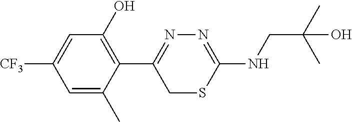 [SMILES: Cc1cc(C(F)(F)F)cc(O)c1C1=NN=C(NCC(C)(C)O)SC1]

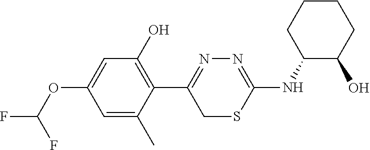 [SMILES: Cc1cc(OC(F)F)cc(O)c1C1=NN=C(N[C@@H]2CCCC[C@H]2O)SC1]

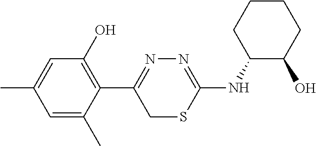 [SMILES: Cc1cc(C)c(C2=NN=C(N[C@@H]3CCCC[C@H]3O)SC2)c(O)c1]

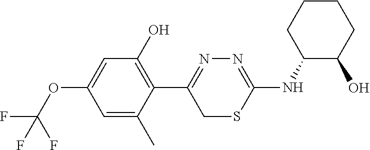 [SMILES: Cc1cc(OC(F)(F)F)cc(O)c1C1=NN=C(N[C@@H]2CCCC[C@H]2O)SC1]

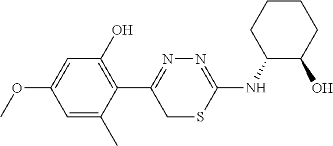 [SMILES: COc1cc(C)c(C2=NN=C(N[C@@H]3CCCC[C@H]3O)SC2)c(O)c1]

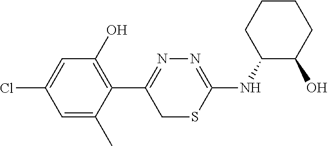 [SMILES: Cc1cc(Cl)cc(O)c1C1=NN=C(N[C@@H]2CCCC[C@H]2O)SC1]

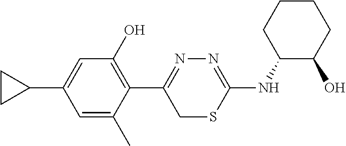 [SMILES: Cc1cc(C2CC2)cc(O)c1C1=NN=C(N[C@@H]2CCCC[C@H]2O)SC1]

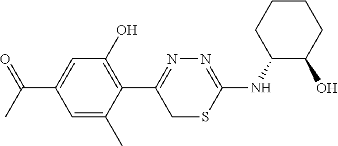 [SMILES: CC(=O)c1cc(C)c(C2=NN=C(N[C@@H]3CCCC[C@H]3O)SC2)c(O)c1]

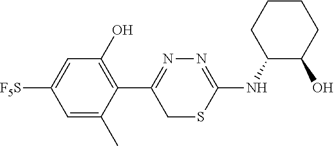

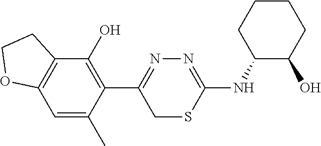 [SMILES: Cc1cc2c(c(O)c1C1=NN=C(N[C@@H]3CCCC[C@H]3O)SC1)CCO2]

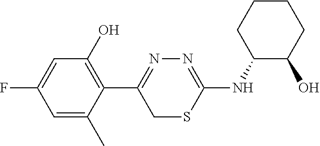 [SMILES: Cc1cc(F)cc(O)c1C1=NN=C(N[C@@H]2CCCC[C@H]2O)SC1]

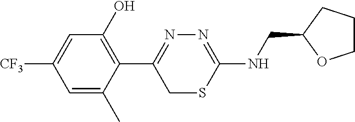 [SMILES: Cc1cc(C(F)(F)F)cc(O)c1C1=NN=C(NC[C@H]2CCCO2)SC1]

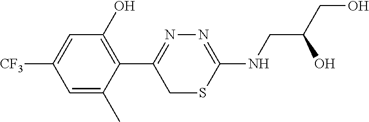 [SMILES: Cc1cc(C(F)(F)F)cc(O)c1C1=NN=C(NC[C@H](O)CO)SC1]

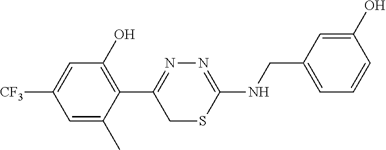 [SMILES: Cc1cc(C(F)(F)F)cc(O)c1C1=NN=C(NCc2cccc(O)c2)SC1]

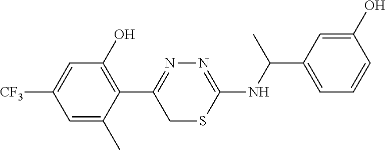 [SMILES: Cc1cc(C(F)(F)F)cc(O)c1C1=NN=C(NC(C)c2cccc(O)c2)SC1]

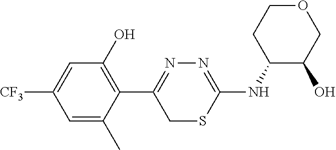 [SMILES: Cc1cc(C(F)(F)F)cc(O)c1C1=NN=C(N[C@@H]2CCOC[C@H]2O)SC1]

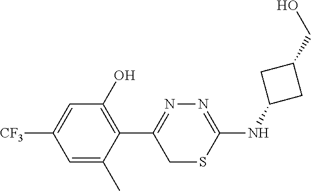 [SMILES: Cc1cc(C(F)(F)F)cc(O)c1C1=NN=C(N[C@H]2C[C@@H](CO)C2)SC1]

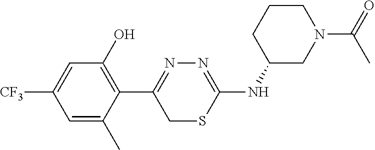 [SMILES: CC(=O)N1CCC[C@@H](NC2=NN=C(c3c(C)cc(C(F)(F)F)cc3O)CS2)C1]

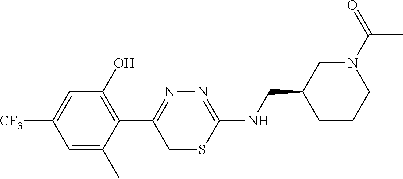 [SMILES: CC(=O)N1CCC[C@@H](CNC2=NN=C(c3c(C)cc(C(F)(F)F)cc3O)CS2)C1]

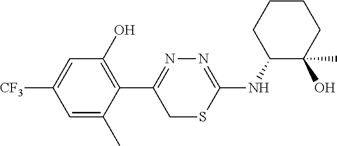 [SMILES: Cc1cc(C(F)(F)F)cc(O)c1C1=NN=C(N[C@@H]2CCCC[C@@]2(C)O)SC1]

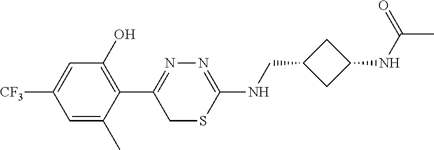 [SMILES: CC(=O)N[C@H]1C[C@@H](CNC2=NN=C(c3c(C)cc(C(F)(F)F)cc3O)CS2)C1]

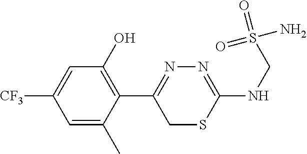 [SMILES: Cc1cc(C(F)(F)F)cc(O)c1C1=NN=C(NCS(N)(=O)=O)SC1]

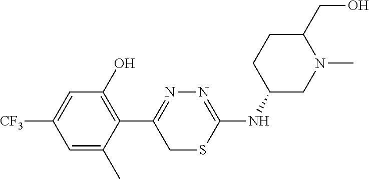 [SMILES: Cc1cc(C(F)(F)F)cc(O)c1C1=NN=C(N[C@@H]2CCC(CO)N(C)C2)SC1]

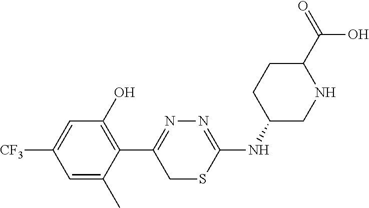 [SMILES: Cc1cc(C(F)(F)F)cc(O)c1C1=NN=C(N[C@@H]2CCC(C(=O)O)NC2)SC1]

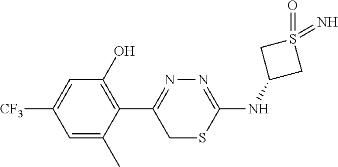

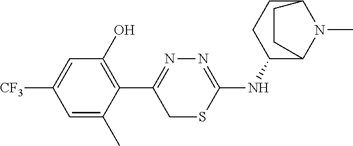 [SMILES: Cc1cc(C(F)(F)F)cc(O)c1C1=NN=C(N[C@@H]2CCC3CCC2N3C)SC1]

### C. Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein include isotopes of hydrogen, carbon, nitrogen, oxygen, phosphorous, sulfur, fluorine, and chloride, such as ²H (D), ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability.

In some embodiments, the abundance of deuterium in each of the substituents disclosed herein is independently at least 1%, at least 10%, at least 20%, at least 30%, at least 40%, at least 50%, at least 60%, at least 70%, at least 80%, at least 90%, or 100% by molar. In some embodiments, one or more of the substituents disclosed herein comprise deuterium at a percentage higher than the natural abundance of deuterium. In some embodiments, one or more 1H are replaced with one or more deuteriums in one or more of the substituents disclosed herein.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or a solvate, or stereoisomer thereof, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein, solvate, or stereoisomer thereof and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. In some embodiments, the disclosure provides for methods of treating diseases by administering the compounds in the form of such solvates. In some embodiments, the disclosure provides for methods of treating diseases by administering a composition comprising the compounds in the form of such solvates. Solvates contain either stoichiometric or non-stoichiometric amounts of a solvent, and, in some embodiments, are formed during the process of crystallization with pharmaceutically acceptable solvents.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond, for example,

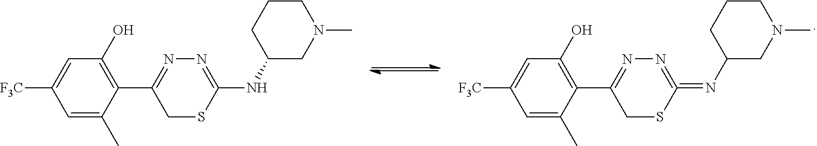

In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Method of Treatment

Disclosed herein is a method of modulating NLRP3 inflammasome in a subject, the method comprising administering to the subject a compound, or a pharmaceutically acceptable salt thereof, disclosed herein. Disclosed herein is a method of inhibiting NLRP3 inflammasome in a subject, the method comprising administering to the subject a compound, or a pharmaceutically acceptable salt thereof, disclosed herein

Disclosed herein are methods of treating a disease modulated at least in part by NLRP3 inflammasome in a subject in need thereof, comprising administering to the subject a therapeutically affective amount of a compound, or a pharmaceutically acceptable salt thereof, disclosed herein.

Disclosed herein is a method of treating an auto-immune or auto-inflammatory disease or condition in a subject in need thereof, the method comprising administering to the subject a therapeutically affective amount of a compound, or a pharmaceutically acceptable salt thereof, disclosed herein.

In some embodiments, the disease or condition is an auto-immune disease.

In some embodiments, the disease or condition is an auto-inflammatory disease.

In some embodiments, the disease or disorder is selected from inflammasome-related diseases/disorders, immune diseases, inflammatory diseases, auto-immune diseases, or auto-inflammatory diseases, for example, autoinflammatory fever syndromes (e.g., cryopyrin-associated periodic syndrome), liver related diseases/disorders (e.g. chronic liver disease, viral hepatitis, non-alcoholic steatohepatitis (NASH), alcoholic steatohepatitis, and alcoholic liver disease), inflammatory arthritis related disorders (e.g. gout, pseudogout (chondrocalcinosis), osteoarthritis, rheumatoid arthritis, arthropathy e.g., acute, chronic), kidney related diseases (e.g. hyperoxaluria, lupus nephritis, Type I/Type II diabetes and related complications (e.g. nephropathy, retinopathy), hypertensive nephropathy, hemodialysis related inflammation), neuroinflammation-related diseases (e.g. multiple sclerosis, brain infection, acute injury, neurodegenerative diseases, Alzheimer's disease), cardiovascular/metabolic diseases/disorders (e.g. cardiovascular risk reduction (CvRR), hypertension, atherosclerosis, type I and type II diabetes and related complications, peripheral artery disease (PAD), acute heart failure), inflammatory skin diseases (e.g. hidradenitis suppurativa, acne), wound healing and scar formation, asthma, sarcoidosis, age-related macular degeneration, and cancer related diseases/disorders (e.g. colon cancer, lung cancer, myeloproliferative neoplasms, leukemias, myelodysplastic syndromes (MDS), myelofibrosis).

In some embodiments, the disease or condition is obesity. In some embodiments, the obesity is induced by high fat diet.

In one aspect, described herein is a method of reducing body weight in a subject in need thereof, comprising administering to the subject a herein disclosed compound or a pharmaceutically acceptable salt or a stereoisomer thereof, or a herein disclosed pharmaceutical composition. In some embodiments, the patient is overweight or obese. In some embodiments, the patient has metabolic disease. In some embodiments, the patient is diabetic or pre-diabetic.

### Dosing

In certain embodiments, the compositions containing the compound(s) described herein are administered for therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation.

### Pharmaceutical Compositions/Formulations

The compounds described herein are administered to a subject in need thereof, either alone or in combination with pharmaceutically acceptable carriers, excipients, or diluents, in a pharmaceutical composition, according to standard pharmaceutical practice. In some embodiments, the compounds described herein are administered to animals.

In another aspect, provided herein are pharmaceutical compositions comprising a compound described herein, or a pharmaceutically acceptable salt thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, (N.Y., 1980; and Pharmaceutical Dosage Forms and Drug Delivery Systems, Seventh Ed. (Lippincott Williams & Wilkins 1999), herein incorporated by reference for such disclosure.

In some embodiments, the pharmaceutically acceptable excipient is selected from carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, and any combinations thereof.

### EXAMPLES

The following examples are offered to illustrate, but not to limit the claimed invention. The following examples further illustrate the invention but, of course, should not be construed as in any way limiting its scope.

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

The compounds and salts of Formulas (A), (B), (I), (Ia), (II), (III*), (III), (IIIa), (IV), (IVa), (V), (Va), or (VIa) can be synthesized according to one or more illustrative schemes herein and/or techniques known in the art. Materials used herein are either commercially available or prepared by synthetic methods generally known in the art. These schemes are not limited to the compounds listed in the examples or by any particular substituents, which are employed for illustrative purposes. Although various steps are described and depicted in the synthesis schemes below, the steps in some cases may be performed in a different order than the order shown below. Numberings or R groups in each scheme do not necessarily correspond to that of the claims or other schemes or tables herein.

### Abbreviations:

| ACN or MeCN | Acetonitrile |
|---|---|
| BAST | Bis(2-methoxyethyl)aminosulfur trifluoride |
| DBU | 1,8-Diaza-7-bicyclo[5.4.0]undecene |
| DCE | 1,2-Dichlorethane |
| DCM | Dichloromethane |
| DDQ | 2,3-Dichloro-5,6-dicyano-1,4-benzoquinone |
| DIAD | Diisopropyl diazodicarboxylate |
| Dioxane | 1,4-dioxane |
| DIPEA or DIEA | diisopropylethylamine |
| DMAP | 4-Dimethylaminopyridine |
| DMF | N,N-Dimethylformamide |
| DMSO | Dimethylsulfoxide |
| EA or EtOAc | Ethyl Acetate |
| ESI | Electrospray ionization |
| FA | Formic acid |
| HOAc or AcOH | Acetic acid |
| HPLC | High Performance Liquid Chromatography |
| KOAc | Potassium acetate |
| Lawesson's Reagent | 2,4-Bis(4-methoxyphenyl)-1,3,2,4- |
| | dithiadiphosphetane-2,4-disulfide |
| LC-MS | Liquid chromatography-mass spectrometry |
| NBS | N-Bromosuccinimide |
| NIS | N-Iodosuccinimide |
| PDC | Bis(pyridinium) dichromate |
| PE | Petroleum ether |
| Prep-HPLC | Preparative-High Performance Liquid |
| | Chromatography |
| Prep-SFC | Preparative-supercritical fluid |
| | chromatography |
| | Prep-TLC |
| Prep-TLC | Preparative-thin layer chromatography |
| rt or RT | room temperature |
| S-Phos | Dicyclohexyl(2′,6′-dimethoxybiphenyl- |
| | 2-yl)phosphine |
| TBSCl or TBS-Cl | tert-Butyldimethylsilyl chloride |
| TEA | Triethylamine |
| TFA | Trifluoroacetic acid |
| THF | Tetrahydrofuran |

### Example 1

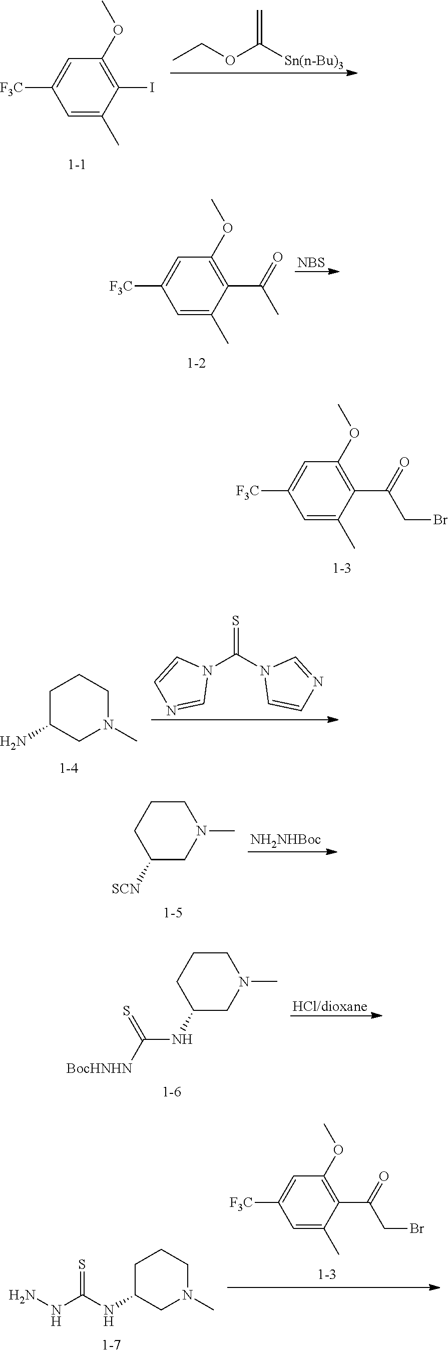

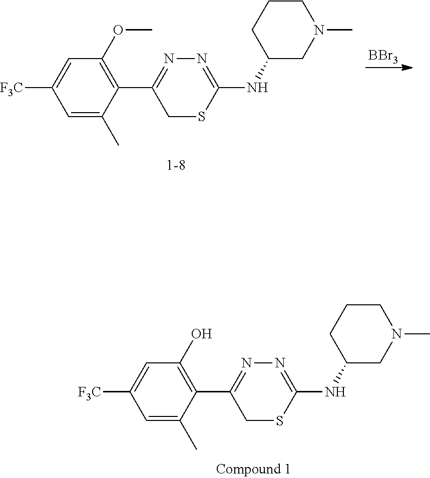

To a solution of compound 1-1 (3 g, 9.49 mmol) and tri(n-butyl)(1-ethoxyvinyl)stannane (3.4 g, 9.41 mmol) in 1,4-dioxane (30 mL) were added CuI (0.2 g, 1.05 mmol) and TEA (1.9 g, 18.78 mmol). The mixture was purged with N₂ for three times. Then Pd(PPh₃)₂Cl₂ (1.1 g, 1.57 mmol) was added. The mixture was purged with N₂ for three times and stirred at 100° C. for 2 hours. After cooling to room temperature, to the reaction mixture was added HCl (10 mL, 6 M) and the resulting solution was stirred for 2 hours. Then, the reaction mixture was poured into H₂O (50 ml) and extracted with ethyl acetate (50 mL×2). The combined organic phase was washed with brine (40 mL), dried over anhydrous Na₂SO₄, filtered through celite, and concentrated under reduced pressure. The residue was purified by flash column chromatography on silica gel eluted with 0˜20% ethyl acetate in petroleum ether to give the intermediate 1-2 (1.2 g, 4.17 mmol, 80.6% purity, 43.9% yield) as a yellow oil. LC-MS (ESI⁺): m/z 233.1 (M+H)⁺.

To a solution of intermediate 1-2 (600 mg, 2.58 mmol) in DCM (5 mL) were added 4-methylbenzenesulfonic acid (667.4 mg, 3.88 mmol) and NBS (689.9 mg, 3.88 mmol). The resulting solution was stirred at 40° C. for 30 min under microwave condition. After cooling to room temperature, the mixture was concentrated under reduced pressure and purified by flash column chromatography on silica gel eluted with 0˜10% ethyl acetate in petroleum ether to give the intermediate 1-3 (600 mg, 0.95 mmol, 49% purity, 36.8% yield) as a yellow oil. LC-MS (ESI⁺): m/z 311.0 (M+H)⁺.

To a solution of compound 1-4 (1.2 g, 10.51 mmol) in DCM (10 mL) was added di(1H-imidazol-1-yl)methanethione (2.1 g, 11.78 mmol) in portions at 0° C. The resulting solution was stirred at 25° C. for 12 hours. The mixture was concentrated under reduced pressure to give the intermediate 1-5 (1.5 g, 9.60 mmol, 91.3% yield) as a white solid, which was used in the next step without further purification.

To a solution of intermediate 1-5 (1.5 g, 9.60 mmol) in acetonitrile (10 mL) were added tert-butyl hydrazinecarboxylate (1.9 g, 14.38 mmol) and TEA (2.66 mL, 19.21 mmol). The resulting solution was stirred at 25° C. for 2 hours. The mixture was concentrated under reduced pressure and purified by flash column chromatography on silica gel eluted with 0˜100% ethyl acetate in petroleum ether to give the intermediate 1-6 (2.5 g, 8.67 mmol, 90.3% yield) as a white solid. LC-MS (ESI⁺): m/z 289.2 (M+H)⁺.

To a solution of intermediate 1-6 (1 g, 3.47 mmol) in 1,4-dioxane (25 mL) was added HCl/1,4-dioxane (25 mL, 4 M, 0.1 mol). The resulting solution was stirred at 25° C. for 4 hours. Then, the mixture was concentrated under reduced pressure to afford the intermediate 1-7 (1.1 g, crude) as a white solid, which was used in the next step without further purification.

To a solution of intermediate 1-7 (240 mg, 1.28 mmol) and intermediate 1-3 (396.5 mg, 1.27 mmol) in EtOH (12 mL) was added TEA (0.21 mL, 1.51 mmol). The resulting solution was stirred at 25° C. for 10 min and at 80° C. for another 1 hour. After cooling to room temperature, the mixture was concentrated under reduced pressure and purified by prep-HPLC (Column: Phenomenex C18 75*30 mm*3 um, Mobile Phase A: water (NH₃H₂O+NH₄HCO₃), Mobile Phase B: acetonitrile, Flow rate: 30 mL/min, gradient condition from 50% B to 80%) and lyophilized to give the intermediate 1-8 (140 mg, 0.35 mmol, 27.4% yield) as a white solid. LC-MS (ESI⁺): m/z 401.2 (M+H)⁺.

To a solution of intermediate 1-8 (160 mg, 0.40 mmol) in DCM (2 mL) was added BBr₃ (0.80 mL) at 0° C. Then the resulting solution was warmed to 25° C. and stirred for 2 hours. After cooling to 0° C., the reaction was quenched with MeOH (1 mL) and stirred at 30° C. for 1 hour. The mixture was concentrated under reduced pressure. The residue was diluted with H₂O (3 mL) and then extracted with EA (3 mL×2). The aqueous phase was adjusted to pH˜9 and then extracted with EA (5 mL×2). The combined organic layers were dried over anhydrous Na₂SO₄, filtered through celite, and concentrated under reduced pressure. The crude product was purified by prep-HPLC (Column: Phenomenex C18 75*30 mm*3 μm, Mobile Phase A: water (NH₃H₂O+NH₄HCO₃), Mobile Phase B: acetonitrile, Flow rate: 30 mL/min, gradient condition from 25% B to 55%) to give the compound 1.

LC-MS (ESI⁺): m/z 387.2 (M+H)⁺.

¹H NMR (400 MHz, DMSO-d₆) 10.46 (br, 1H), 7.09 (s, 1H), 7.07-6.73 (m, 2H), 4.06-3.91 (m, 1H), 3.41-3.38 (m, 2H), 3.02-2.92 (m, 1H), 2.69-2.60 (m, 1H), 2.29 (s, 3H), 2.20 (s, 3H), 1.94-1.77 (m, 3H), 1.72-1.65 (m, 1H), 1.57-1.47 (m, 1H), 1.32-1.21 (m, 1H).

### Example 2

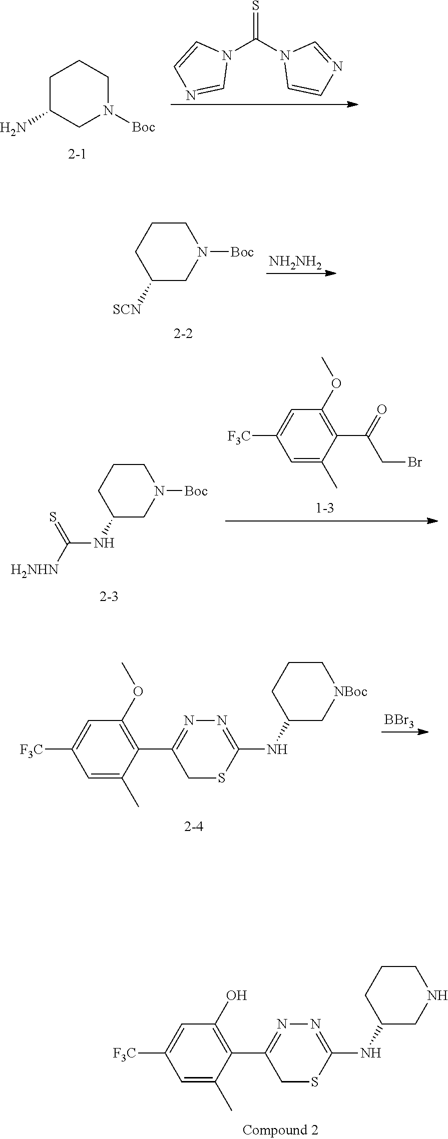

To a solution of compound 2-1 (6 g, 29.96 mmol) in DCM (80 mL) was added di(1H-imidazol-1-yl)methanethione (5871.8 mg, 32.95 mmol) in portions and the resulting solution was stirred at 25° C. for 12 hours. The mixture was concentrated under reduced pressure and purified by flash column chromatography on silica gel eluted with 0˜60% ethyl acetate in petroleum ether to give the intermediate 2-2 (6.2 g, 25.58 mmol, 85.4% yield) as yellow oil.

¹H NMR (400 MHz, CDCl₃) δ=3.77-3.24 (m, 5H), 1.99-1.72 (m, 3H), 1.60-1.45 (m, 10H)

To a solution of intermediate 2-2 (2 g, 8.25 mmol) in acetonitrile (20 mL) was added hydrazine (0.49 mL, 12.38 mmol) at 0° C. The reaction mixture was stirred at 25° C. for 2 hours. The reaction mixture was concentrated under reduced pressure to give the intermediate 2-3 (2.2 g, 8.02 mmol, 97.2% yield) as white solid. LC-MS (ESI⁺): m/z 275.2 (M+H)⁺.

A solution of intermediate 2-3 (278 mg, 1.01 mmol), intermediate 1-3 (598.8 mg, 1.92 mmol) and TEA (0.17 mL, 1.22 mmol) in EtOH (3 mL) was stirred at 25° C. for 10 min, then the resulting solution was heated to 80° C. for 2 hours. After cooling to room temperature, the mixture was concentrated under reduced pressure and purified by Prep-HPLC (Welch Xtimate C18 150*25 mm*5 μm, Mobile Phase A: water (FA), Mobile Phase B: acetonitrile, Flow rate: 25 mL/min, gradient condition from 30% B to 60%) to give the intermediate 2-4 (25 mg, 0.026 mmol, 50% purity, 2.5% yield) as white solid. LC-MS (ESI⁺): m/z 487.1 (M+H)⁺.

To a solution of intermediate 2-4 (25 mg, 0.051 mmol) in DCM (2 mL) was added BBr₃ (0.10 mL, 1M in THF, 0.1 mmol) at 0° C. The resulting solution was stirred at 25° C. for 2 hours. The reaction was quenched with MeOH (1 mL) and then concentrated under reduced pressure. The residue was purified by prep-HPLC (Column: Boston Green ODS 150*30 mm*5 μm, Mobile Phase A: water (FA), Mobile Phase B: acetonitrile, Flow rate: 40 mL/min, gradient condition from 10% B to 40%) and lyophilized to give the compound 2. LC-MS (ESI⁺): m/z 373.2 (M+H)⁺. ¹H NMR (400 MHz, CD₃OD) δ=8.53 (br, 1H from HCOOH), 7.07 (s, 1H), 6.98 (s, 1H), 4.30-4.16 (m, 1H), 3.67-3.58 (m, 1H), 3.54-3.44 (m, 2H), 3.30-3.25 (m, 1H), 3.04-2.89 (m, 2H), 2.37 (s, 3H), 2.17-2.01 (m, 2H), 1.89-1.77 (m, 1H), 1.74-1.63 (m, 1H)

### Example 3

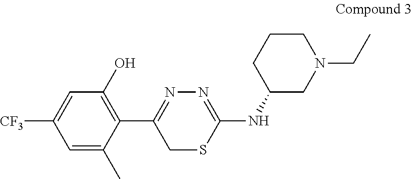 [SMILES: CCN1CCC[C@@H](NC2=NN=C(c3c(C)cc(C(F)(F)F)cc3O)CS2)C1]

In a similar fashion according to the procedure for Compound 1, Compound 3 was synthesized by replacing intermediate 1-4 with (R)-1-ethylpiperidin-3-amine. The crude product was purified by prep-HPLC (column: Welch Xtimate C18 150*25 mm*5 um, water (NH₃H₂O+NH₄HCO₃)-ACN]; B %: 40%-70%, 7 min) to give the compound 3.

LCMS (ESI+): m/z 401.2 (M+H)⁺.

¹HNMR: (400 MHz, DMSO-d₆) δ=10.98-9.83 (m, 1H), 7.07 (s, 1H), 7.01 (s, 1H), 4.05-3.87 (m, 1H), 3.43-3.35 (m, 2H), 3.11-2.96 (m, 1H), 2.81-2.67 (m, 1H), 2.41-2.27 (m, 5H), 1.97-1.76 (m, 3H), 1.73-1.62 (m, 1H), 1.55-1.43 (m, 1H), 1.34-1.21 (m, 1H), 1.00 (t, J=7.2 Hz, 3H).

### Example 4

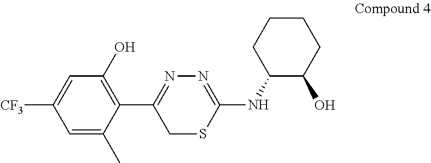 [SMILES: Cc1cc(C(F)(F)F)cc(O)c1C1=NN=C(N[C@@H]2CCCC[C@H]2O)SC1]

In a similar fashion according to the procedure tor Compound 1, Compound 4 was synthesized by replacing intermediate 1-4 with (1R,2R)-2-(benzyloxy)cyclohexan-1-amine. The crude product was purified by prep-HPLC (Column: Welch Xtimate C18 150*25 mm*5 μm, Mobile Phase A: water (NH₃H₂O+NH₄HCO₃), Mobile Phase B: acetonitrile, Flow rate: 25 mL/min, gradient condition from 40% B to 70%) and lyophilized to give the compound 4.

LC-MS (ESI+): m/z 388.2 (M+H)⁺.

¹H NMR (400 MHz, ACETONITRILE-d₃) δ=7.10 (s, 1H), 7.05 (s, 1H), 3.73-3.65 (m, 1H), 3.59-3.44 (m, 2H), 3.41-3.33 (m, 1H), 2.40 (s, 3H), 2.07-1.98 (m, 2H), 1.73-1.65 (m, 2H), 1.36-1.23 (m, 4H).

### Example 5

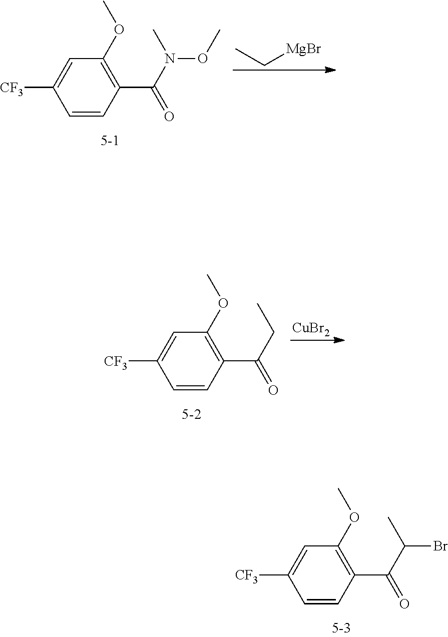

To a solution of compound 5-1 (1.2 g, 4.56 mmol) in THF (12 mL) was added ethylmagnesium bromide (1 M in THF, 4.56 mL, 4.56 mmol) dropwise at 0° C., and the mixture was stirred at 20° C. for 12 hours. The volatiles were removed in vacuo. The residue was adjusted to pH=5 with aqueous HCl solution (2 M) and extracted with ethyl acetate (200 mL×2). The combined organic extracts were washed with brine (80 mL), dried over anhydrous Na₂SO₄, filtered through a celite pad and concentrated under reduced pressure to give the intermediate 5-2 (828.0 mg, 69.6% purity, 54.4% yield) as yellow oil. LC-MS (ESI⁺): m/z 233.1 (M+H)⁺.

A solution of intermediate 5-2 (800.0 mg, 3.45 mmol) in DCE (4 mL) was added to a mixture of CuBr₂ (1539.0 mg, 6.89 mmol) in ethyl acetate (12 mL) at 80° C. and the reaction mixture was stirred at 80° C. for 3 hours. After cooling to room temperature, the volatiles were removed in vacuo. The residue was poured into water (100 mL) and extracted with ethyl acetate (80 mL×3). The combined organic extracts were washed with brine (50 mL), dried over anhydrous Na₂SO₄, filtered through a celite pad and the filtrate was concentrated under reduced pressure to give crude product. Then it was purified by flash column chromatography on silica gel eluted with 0˜15% ethyl acetate in petroleum ether to give the intermediate 5-3 (829 mg, 89.1% purity, 68.8% yield) as orange oil. LC-MS (ESI⁺): m/z 311.0 (M+H)⁺.

### Example 6

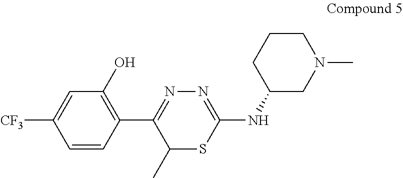 [SMILES: CC1SC(N[C@@H]2CCCN(C)C2)=NN=C1c1ccc(C(F)(F)F)cc1O]

In a similar fashion according to the procedure for Compound 1, Compound 5 was synthesized by replacing intermediate 1-3 with intermediate 5-3. The crude product was purified by prep-HPLC (Column: Phenomenex C18 75*30 mm*3 μm, Mobile Phase A: water (NH₃H₂O+NH₄HCO₃), Mobile Phase B: acetonitrile, Flow rate: 25 mL/min, gradient condition from 55% B to 85%) and lyophilized to give the compound 5. LC-MS (ESI⁺): m/z 387.2 (M+H)⁺.

¹H NMR (400 MHz, DMSO-d₆) δ=14.99-14.88 (m, 1H), 7.85 (d, J=8.4 Hz, 1H), 7.68-7.53 (m, 1H), 7.25-7.19 (m, 2H), 4.84-4.76 (m, 1H), 4.17-3.97 (m, 1H), 3.06-2.76 (m, 1H), 2.65-2.55 (m, 1H), 2.19-2.16 (m, 3H), 1.96-1.75 (m, 3H), 1.72-1.64 (m, 1H), 1.58-1.46 (m, 1H), 1.32-1.22 (m, 4H).

### Example 7

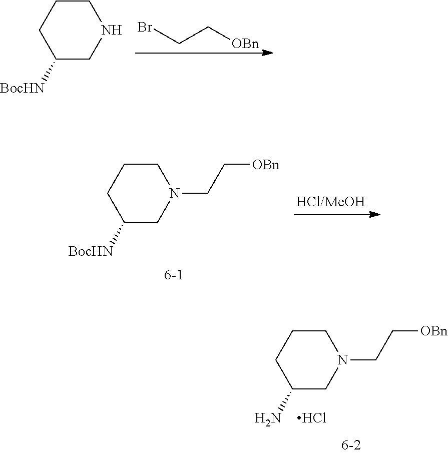

To a solution of (R)-tert-butyl piperidin-3-ylcarbamate (2.00 g, 9.99 mmol) and ((2-bromoethoxy)methyl)benzene (2.14 g, 9.99 mmol) in MeCN (20 mL) was added K₂CO₃ (2.76 g, 20.0 mmol). The resulting mixture was stirred at 60° C. for 1 hour. After cooling to room temperature, the mixture was filtered through a celite pad and the filtrate was concentrated under reduced pressure to afford a residue. The residue was purified by flash column chromatography on silica gel eluted with 0˜100% ethyl acetate in petroleum ether to give the intermediate 6-1 (2.16 g, 64.7% yield) as white solid. LC-MS (ESI⁺): m/z 335.3 (M+H)⁺.

To a solution of intermediate 6-1 (500.0 mg, 1.49 mmol) in MeOH (2.0 mL) was added HCl/MeOH (2.0 mL, 4 M) and the resulting mixture was stirred at 20° C. for 12 hours. The reaction mixture was concentrated under reduced pressure to give the intermediate 6-2 (570.0 mg, crude) as a white solid. LC-MS (ESI⁺): m/z 235.2 (M+H)⁺.

### Example 8

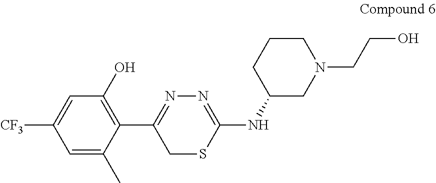 [SMILES: Cc1cc(C(F)(F)F)cc(O)c1C1=NN=C(N[C@@H]2CCCN(CCO)C2)SC1]

In a similar fashion according to the procedure for Compound 1, Compound 6 was synthesized by replacing intermediate 1-4 with intermediate 6-2. The crude product was purified by HPLC column: Welch Xtimate C18 150*25 mm*5 μm, water (NH₃H₂O+NH₄HCO₃)-ACN]; B %: 30%-60%, 7 min. to give the compound 6.

LC-MS (ESI⁺): m/z 417.2 (M+H)⁺.

¹H NMR (400 MHz, DMSO-d₆) δ=7.05 (s, 1H), 6.99 (s, 1H), 4.54-4.26 (m, 1H), 4.09-3.90 (m, 1H), 3.49-3.46 (m, 4H), 3.01-2.92 (m, 1H), 2.71-2.62 (m, 1H), 2.39 (t, J=6.0 Hz, 2H), 2.29 (s, 3H), 2.06-1.92 (m, 2H), 1.86-1.75 (m, 1H), 1.72-1.60 (m, 1H), 1.56-1.42 (m, 1H), 1.37-1.25 (m, 1H).

### Example 9

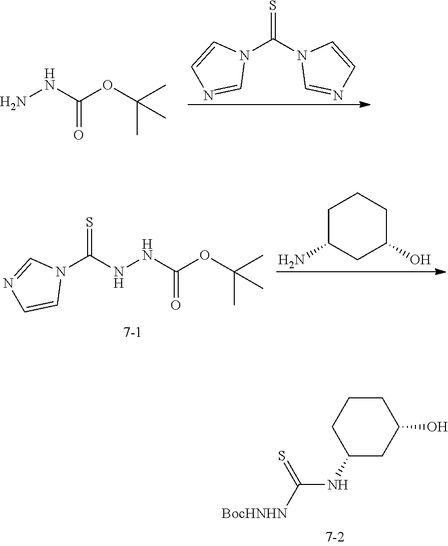

To a solution of di(1H-imidazol-1-yl)methanethione (1.00 g, 5.61 mmol) in dioxane (10 mL) was slowly added tert-butyl hydrazinecarboxylate (700.0 mg, 5.50 mmol) and the reaction mixture was stirred at 25° C. for 1 hour. The reaction mixture was poured into water (20 mL) and extracted with ethyl acetate (20 mL×3). The combined organic extracts were washed with brine (10 mL), dried over anhydrous Na₂SO₄, filtered through a celite pad and the filtrate was concentrated under reduced pressure to give a residue. The residue was purified by flash column chromatography on silica gel eluted with 0˜100% ethyl acetate in petroleum ether to give the intermediate 7-1 (1.00 g, 73.3% yield) as yellow solid.

LC-MS (ESI+): m/z 243.0 (M+H)⁺.

¹H NMR (400 MHz, DMSO-d₆) δ=8.82 (s, 1H), 8.52 (s, 1H), 7.80 (s, 1H), 7.43 (s, 1H), 7.22 (s, 1H), 1.44 (s, 9H).

To a solution of intermediate 7-1 (552.1 mg, 2.28 mmol) and (1S,3R)-3-aminocyclohexan-1-ol hydrochloride (380.0 mg, 2.51 mmol) in dioxane (5.0 mL) was added TEA (0.95 mL, 6.84 mmol). The resulting mixture was stirred at 80° C. for 1 hour. After cooling to room temperature, the mixture was concentrated under reduced pressure to afford a residue. The residue was purified by flash column chromatography on silica gel eluted with 0˜100% ethyl acetate in petroleum ether to give the intermediate 7-2 (300.0 mg, 97.7% purity, 44.4% yield) as a white solid. LC-MS (ESI⁺): m/z 290.1 (M+H)⁺.

### Example 10

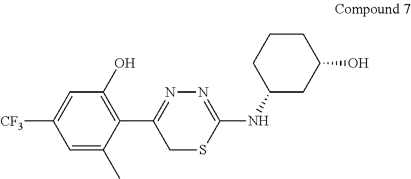 [SMILES: Cc1cc(C(F)(F)F)cc(O)c1C1=NN=C(N[C@@H]2CCC[C@H](O)C2)SC1]

In a similar fashion according to the procedure for Compound 1, Compound 7 was synthesized by replacing intermediate 1-6 with intermediate 7-2. The crude product was purified by Prep-HPLC (Column: Welch Xtimate C₁₈ 150*25 mm*5 um, Mobile Phase A: water (NH₄HCO₃), Mobile Phase B: acetonitrile, Flow rate: 25 mL/min, gradient condition from 35% B to 65%) and lyophilized to give the compound 7.

LC-MS (ESI+): m/z 388.2 (M+H)⁺.

¹H NMR (400 MHz, DMSO-d₆) δ=10.47 (br. s., 1H), 7.08 (s, 1H), 7.01 (s, 1H), 4.69-4.60 (m, 1H), 3.91-3.73 (m, 1H), 3.50-3.37 (m, 3H), 2.29 (s, 3H), 2.19-2.10 (m, 1H), 1.92-1.84 (m, 1H), 1.83-1.75 (m, 1H), 1.72-1.65 (m, 1H), 1.31-1.01 (m, 4H).

### Example 11

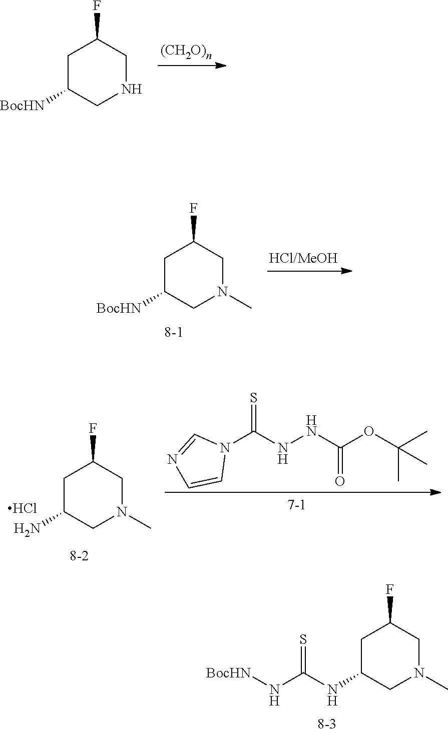

To a solution of (3R,5R)-tert-butyl (5-fluoropiperidin-3-yl) carbamate (2.0 g, 9.16 mmol) and (CH₂O)ₙ (1.0 g) in MeOH (20 mL) was added HOAc (0.52 mL, 9.16 mmol). Then, the mixture was stirred at 25° C. for 0.5 hours. NaBH₃CN (2.65 g, 42.14 mmol) was added and the resulting mixture was stirred at 25° C. for 5 hours. The mixture was filtered through a celite pad and the filtrate was concentrated under reduced pressure to afford the crude product and it was purified by Prep-HPLC (column: Welch Xtimate C18 150*25 mm*5 μm, water (NH₃H₂O+NH₄HCO₃)-ACN]; B %: 25%-55%, 7 min) to give the intermediate 8-1 (1.88 g, 88.3% yield) as a white solid.

LC-MS (ESI+): m/z 233.2 (M+H)⁺.

To a solution of intermediate 8-1 (900.0 mg, 3.87 mmol) in MeOH (3.0 mL) was added 4M HCl in MeOH (2.0 mL) and the resulting solution was stirred at 20° C. for 12 hours. The volatiles were removed in vacuo to give the intermediate 8-2 (688 mg, crude) as a white solid.

A mixture of intermediate 8-2 (588.0 mg, 3.49 mmol), intermediate 7-1 (929.0 mg, 3.83 mmol) and TEA (0.48 mL, 3.49 mmol) in dioxane (6.0 mL) was stirred at 80° C. for 1 hour. The reaction mixture was filtered through a celite pad and the filtrate was concentrated under reduced pressure to give a residue. Then, it was purified by flash column chromatography on silica gel eluted with 0˜100% ethyl acetate in petroleum ether to give the intermediate 8-3 (210.0 mg, 19.7% yield) as yellow solid. LC-MS (ESI⁺): m/z 307.1 (M+H)⁺.

### Example 12

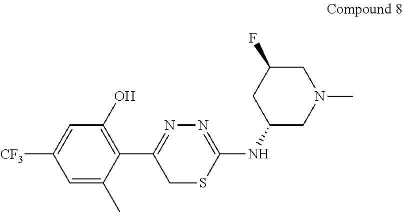 [SMILES: Cc1cc(C(F)(F)F)cc(O)c1C1=NN=C(N[C@@H]2C[C@@H](F)CN(C)C2)SC1]

In a similar fashion according to the procedure for Compound 1, Compound 8 was synthesized by replacing intermediate 1-6 with intermediate 8-3. The crude product was purified by Prep-HPLC (column: Welch Xtimate C18 150*25 mm*5 μm, water (NH₃H₂O+NH₄HCO₃)-ACN]; B %: 42%-72%, 7 min) and lyophilized to give the compound 8.

LC-MS (ESI+): m/z 405.2 (M+H)⁺.

¹H NMR (400 MHz, DMSO-d₆) δ=10.36 (br. s., 1H), 7.07 (s, 1H), 7.01 (s, 1H), 5.11-4.74 (m, 1H), 4.40-4.12 (m, 1H), 3.44-3.37 (m, 2H), 2.98-2.88 (m, 1H), 2.87-2.75 (m, 1H), 2.29 (s, 3H), 2.20 (s, 3H), 2.17-2.00 (m, 2H), 1.95-1.85 (m, 1H), 1.71-1.46 (m, 1H).

### Example 13

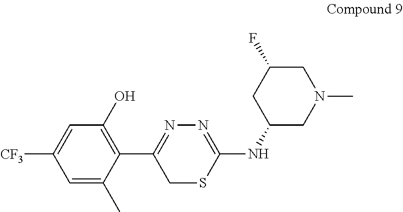 [SMILES: Cc1cc(C(F)(F)F)cc(O)c1C1=NN=C(N[C@@H]2C[C@H](F)CN(C)C2)SC1]

In a similar fashion according to the procedure for Compound 8, Compound 9 was synthesized by replacing tert-butyl ((3R,5R)-5-fluoropiperidin-3-yl) carbamate with tert-butyl ((3R,5S)-5-fluoropiperidin-3-yl) carbamate. The crude product was purified by Prep-HPLC (column: Welch Xtimate C18 150*30 mm*5 um, water (FA)-ACN]; B %: 10%-40%, 7 min) and lyophilized to give the compound 9.

LC-MS (ESI+): m/z 405.2 (M+H)⁺.

¹H NMR (400 MHz, DMSO-d₆) δ=11.27-9.92 (m, 1H), 8.18 (s, 0.46H from HCOOH), 7.08 (s, 1H), 7.01 (s, 1H), 4.78-4.56 (m, 1H), 4.11-3.95 (m, 1H), 3.41-3.37 (m, 2H), 3.08-2.91 (m, 2H), 2.36-2.26 (m, 4H), 2.25 (s, 3H), 1.98-1.88 (m, 1H), 1.84-1.69 (m, 1H), 1.53-1.37 (m, 1H).

### Example 14

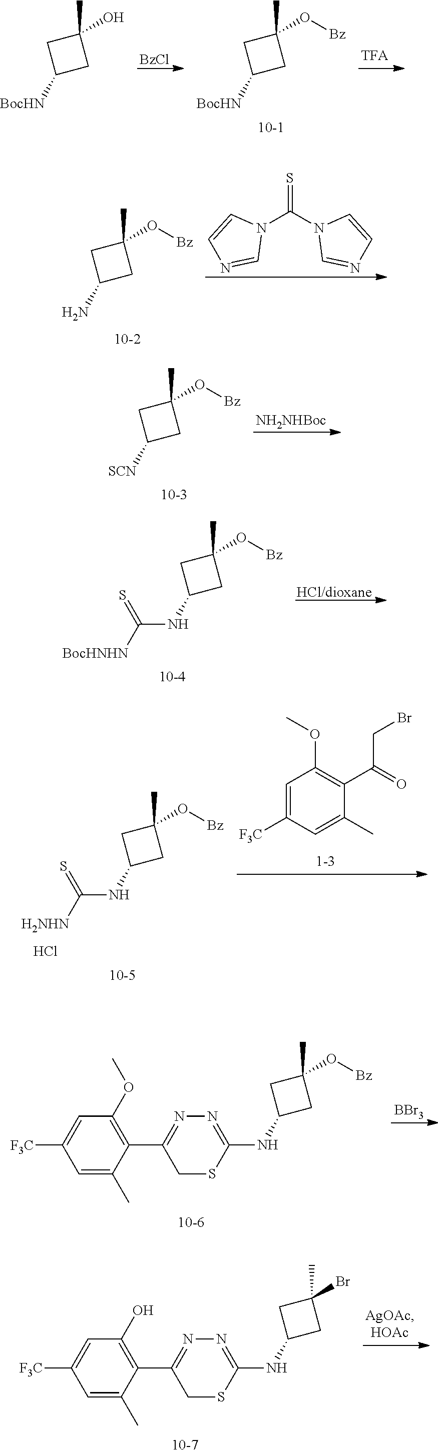

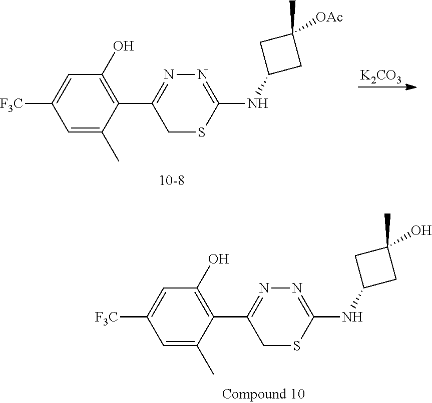

To a mixture of tert-butyl ((1s,3s)-3-hydroxy-3-methylcyclobutyl) carbamate (1.00 g, 4.97 mmol), benzoyl chloride (0.58 mL, 4.97 mmol) and TEA (0.69 mL, 4.97 mmol) in DCM (15.0 mL) was added DMAP (607.2 mg, 4.97 mmol) and the mixture was stirred at 25° C. for 12 hour. Then, the reaction mixture was concentrated under reduced pressure to give a residue. The residue was purified by flash column chromatography on silica gel eluted with 0˜25% ethyl acetate in petroleum ether to give the intermediate 10-1 (408.3 mg, 61.4% purity, 16.5% yield) as a white solid. LC-MS (ESI+): m/z: 328.1 (M+Na)⁺.

To a solution of intermediate 10-1 (440.0 mg, 1.44 mmol) in DCM (5.0 mL) was added TFA (1.0 mL, 13.06 mmol) and the mixture was stirred at 20° C. for 1 hour. The reaction mixture was concentrated under reduced pressure to give a residue. The residue was adjusted to pH=8-9 with TEA and diluted with ethyl acetate (20 mL). The mixture was filtered and the filtrate was concentrated under reduced pressure to give the intermediate 10-2 (300.0 mg, crude) as brown oil. LC-MS (ESI+): m/z: 206.2 (M+H)⁺.

To a solution of intermediate 10-2 (300.0 mg, crude) in DCM (10.0 mL) was added di(1H-imidazol-1-yl)methanethione (286.4 mg, 1.61 mmol) in portions at 0° C. and the mixture was stirred at 25° C. for 12 hours. The reaction mixture was concentrated under reduced pressure to give the intermediate 10-3 (350.0 mg, crude) as brown oil, which was used in the next step without further purification.

To a solution of intermediate 10-3 (350.0 mg, crude) in MeCN (5.0 mL) was added tert-butyl hydrazinecarboxylate (374.2 mg, 2.83 mmol) and TEA (0.20 mL, 1.42 mmol), and the reaction mixture was stirred at 25° C. for 2 hours. The reaction mixture was concentrated under reduced pressure to give a residue. The residue was purified by flash column chromatography on silica gel eluted with 0˜25% ethyl acetate in petroleum ether to give the intermediate 10-4 (552.6 mg, 97.7% purity, 98.8% yield over 3 steps) as a white solid. LC-MS (ESI+): m/z: 380.2 (M+H)⁺.

To a solution of intermediate 10-4 (150.0 mg, 0.40 mmol) in dioxane (5.0 mL) was added HCl/dioxane (3.0 mL, 4 M). The mixture was stirred at 20° C. for 1.5 hour. The volatiles were removed in vacuo to give the intermediate 10-5 (110.0 mg, crude) as a white solid, which was used in the next step without further purification.

A solution of intermediate 10-5 (110.0 mg, crude), intermediate 1-3 (122.5 mg, 0.39 mmol) and TEA (50 μL, 0.36 mmol) in EtOH (3.0 mL) was stirred at 25° C. for 10 minutes and at 80° C. for another 1 hour. After cooling to room temperature, the mixture was concentrated under reduced pressure to afford a residue. The residue was poured into water (100 mL) and extracted with ethyl acetate (80 mL×3). The combined organic extracts were washed with brine (50 mL), dried over anhydrous Na₂SO₄, filtered through a celite pad and the filtrate was concentrated under reduced pressure to give the intermediate 10-6 (150.0 mg, crude) as yellow oil. LC-MS (ESI+): m/z: 492.2 (M+H)⁺.

To a solution of intermediate 10-6 (110.0 mg, 0.22 mmol) in DCM (2.0 mL) was added BBr₃ (0.22 mL, 1M in DCM, 0.22 mmol) dropwise at 0° C. and the mixture was stirred at 20° C. for 1 hour. The reaction mixture was quenched by adding MeOH (5.0 mL) slowly and the volatiles were removed in vacuo to give the residue. The residue was poured into water (20 mL) and extracted with ethyl acetate (30 mL×3). The combined organic extracts were washed with brine (15 mL), dried over anhydrous Na₂SO₄, filtered through a celite pad and the filtrate was concentrated under reduced pressure to give the crude. The crude was purified by Prep-HPLC (column: Phenomenex C18 80*45 mm*3 μm, mobile phase: water (NH₃H₂O+NH₄HCO₃)-ACN; B %: 48%-78%, 9 min) and lyophilized to give the intermediate 10-7 (30.0 mg, 88.2% purity, 27.6% yield) as a yellow solid. LC-MS (ESI⁺): m/z: 436.1 (M+H)⁺.

A mixture of intermediate 10-7 (15.0 mg, 0.034 mmol) and silver acetate (14.3 mg, 0.086 mmol) in acetic acid (2.0 mL) was stirred at 20° C. for 12 hour. The mixture was filtered through a celite pad and the filtrate was lyophilized to give the intermediate 10-8 (15.0 mg, crude) as a brown solid. LC-MS (ESI⁺): m/z: 416.1 (M+H)⁺.

To a solution of intermediate 10-8 (20.0 mg, 0.048 mmol) in MeOH (2.0 mL) was added K₂CO₃ (6.7 mg, 0.048 mmol). The mixture was stirred at 20° C. for 0.5 hour. Then, the mixture was filtered through a celite pad and the filtrate was concentrated under reduced pressure to give a residue. The residue was purified by Prep-HPLC (Column: Phenomenex C18 75*30 mm*3 um, Mobile Phase A: water (NH₃H₂O+NH₄HCO₃), Mobile Phase B: acetonitrile, Flow rate: 25 mL/min, gradient condition from 23% B to 53%) to give the compound 10.

LC-MS (ESI⁺): m/z: 374.1 (M+H)⁺.

¹H NMR (400 MHz, DMSO-d₆) δ=10.93-10.08 (m, 1H), 7.08 (s, 1H), 7.02 (s, 1H), 5.01 (s, 1H), 4.01-3.90 (m, 1H), 3.47-3.40 (m, 2H), 2.35-2.30 (m, 2H), 2.28 (s, 3H), 2.06-1.99 (m, 2H), 1.25 (s, 3H).

### Example 15

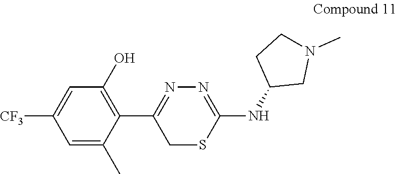 [SMILES: Cc1cc(C(F)(F)F)cc(O)c1C1=NN=C(N[C@@H]2CCN(C)C2)SC1]

In a similar fashion according to the procedure for Compound 1, Compound 11 was synthesized by replacing intermediate 1-4 with (R)-1-methylpyrrolidin-3-amine. The crude product was purified by Prep-HPLC (Column: Phenomenex C18 75*30 mm*3 μm, Mobile Phase A: water (NH₃H₂O+NH₄HCO₃), Mobile Phase B: acetonitrile, Flow rate: 25 mL/min, gradient condition from 35% B to 65%) to give the compound 11.

LC-MS (ESI+): m/z: 373.2 (M+H)⁺.

¹H NMR (400 MHz, DMSO-d₆) δ=11.05-9.91 (m, 1H), 7.07 (s, 1H), 7.00 (s, 1H), 4.45-4.31 (m, 1H), 3.42-3.38 (m, 2H), 2.76-2.68 (m, 1H), 2.62-2.54 (m, 1H), 2.48-2.43 (m, 1H), 2.42-2.35 (m, 1H), 2.28 (s, 3H), 2.25 (s, 3H), 2.21-2.11 (m, 1H), 1.75-1.65 (m, 1H).

### Example 16

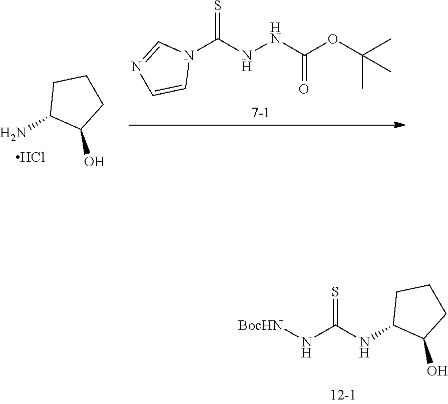 [SMILES: N[C@@H]1CCC[C@H]1O ; Cl ; CC(C)(C)OC(=O)NNC(=S)n1ccnc1 ; CC(C)(C)OC(=O)NNC(=S)N[C@@H]1CCC[C@H]1O]

To a solution of (1R,2R)-2-aminocyclopentan-1-ol hydrochloride (400.0 mg, 2.91 mmol) and TEA (1.21 mL, 8.72 mmol) in dioxane (10.0 mL) was added tert-butyl 2-(1H-imidazole-1-carbonothioyl) hydrazine-1-carboxylate (1000.0 mg, 4.13 mmol) at 20° C. and the mixture was stirred at 110° C. for 1 hour. After cooling to room temperature, the mixture was concentrated under reduced pressure to afford a residue. The residue was purified by flash column chromatography on silica gel eluted with 0˜24% THF in DCM to give the intermediate 12-1 (450.0 mg, 94.8% purity, 53.3% yield) as white solid. LC-MS (ESI+): m/z: 297.9 (M+Na)⁺.

### Example 17

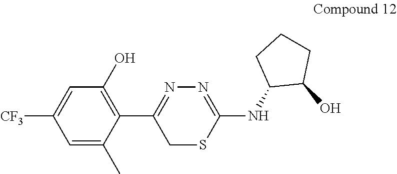 [SMILES: Cc1cc(C(F)(F)F)cc(O)c1C1=NN=C(N[C@@H]2CCC[C@H]2O)SC1]

In a similar fashion according to the procedure for Compound 1, Compound 12 was synthesized by replacing intermediate 1-6 with intermediate 12-1. The residue was purified by Prep-HPLC (Column: Phenomenex C18 80*40 mm*3 μm, Mobile Phase A: water (NH₃H₂O+NH₄HCO₃), Mobile Phase B: acetonitrile, Flow rate: 30 mL/min, gradient condition from 32% B to 62%) to give the compound 12.

LC-MS (ESI+): m/z: 374.1 (M+H)⁺.

¹H NMR (400 MHz, DMSO-d₆) δ=10.46 (br. s., 1H), 7.08 (s, 1H), 7.01 (s, 1H), 5.38-5.05 (m, 1H), 3.98-3.89 (m, 2H), 3.43-3.38 (m, 2H), 2.29 (s, 3H), 2.09-1.98 (m, 1H), 1.90-1.81 (m, 1H), 1.70-1.59 (m, 2H), 1.55-1.42 (m, 2H).

### Example 18

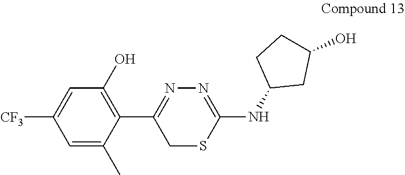 [SMILES: Cc1cc(C(F)(F)F)cc(O)c1C1=NN=C(N[C@@H]2CC[C@H](O)C2)SC1]

In a similar fashion according to the procedure for Compound 12, Compound 13 was synthesized by replacing (1R,2R)-2-aminocyclopentan-1-ol with (1S,3R)-3-aminocyclopentan-1-ol. The crude product was purified by Prep-HPLC (Column: Welch Xtimate C18 150*30 mm*5 um, Mobile Phase: water (FA)-ACN) and lyophilized to give the compound 13.

LC-MS (ESI+): m/z 374.2 (M+H)⁺.

¹H NMR (400 MHz, DMSO-d₆) δ=8.13 (s, 1H from HCOOH), 7.08 (s, 1H), 7.01 (s, 1H), 5.01-4.45 (m, 1H), 4.27-4.14 (m, 1H), 4.13-4.01 (m, 1H), 3.43-3.40 (m, 2H), 2.29 (s, 3H), 2.25-2.15 (m, 1H), 1.99-1.84 (m, 1H), 1.78-1.54 (m, 3H), 1.52-1.43 (m, 1H).

### Example 19

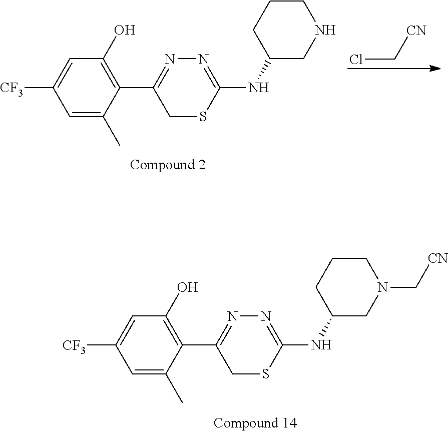

To a solution of compound 2 (150.0 mg, 0.23 mmol, 70% purity) and DIPEA (90 μL, 0.56 mmol) in THF (5.0 mL) was added 2-chloroacetonitrile (21.3 mg, 0.28 mmol) and the reaction mixture was stirred at 67° C. for 2 hours. After cooling to room temperature, the volatiles were removed in vacuo to afford a residue. The residue was purified by Prep-HPLC (Column: Phenomenex Gemini-NX 80*40 mm*3 um, Mobile Phase A: water (0.05% NH₃H₂O+10 mM NH₄HCO₃), Mobile Phase B: acetonitrile, Flow rate: 30 mL/min, gradient condition from 30% B to 60%), then by Prep-SFC (Column: DAICEL CHIRALCEL OJ (250 mm*30 mm, 10 μm), Condition: CO₂-EtOH (0.1% NH₃H₂O), Begin B: 30%, Flow Rate: 70 ml/min) and lyophilized to give the compound 14.

LC-MS (ESI+): m/z 412.2 (M+H)⁺.

¹H NMR (400 MHz, DMSO-d₆) δ=10.50 (br. s, 1H), 7.07 (s, 1H), 7.00 (s, 1H), 4.10-3.93 (m, 1H), 3.77 (s, 2H), 3.42-3.37 (m, 2H), 3.06-2.96 (m, 1H), 2.71-2.64 (m, 1H), 2.28 (s, 3H), 2.18-2.02 (m, 2H), 1.89-1.82 (m, 1H), 1.78-1.70 (m, 1H), 1.59-1.49 (m, 1H), 1.31-1.22 (m, 1H).

### Example 20

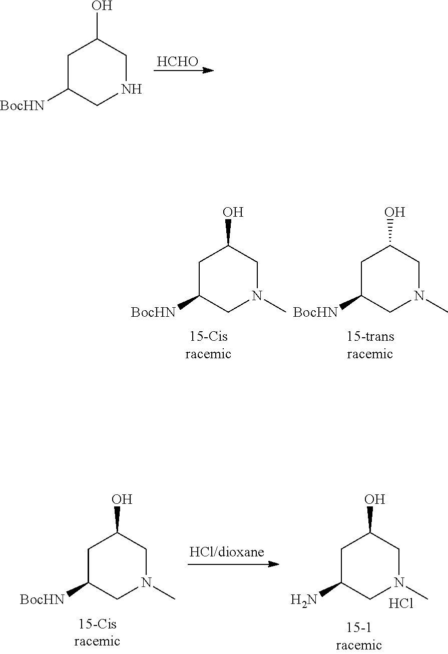

To a solution of tert-butyl (5-hydroxypiperidin-3-yl) carbamate (900.0 mg, 4.16 mmol) and (CH₂O)ₙ (900.0 mg, 29.97 mmol) in MeOH (3.0 mL) was added HOAc (0.24 mL, 4.16 mmol) and the mixture was stirred at 25° C. for 30 min. Then NaBH₃CN (653.3 mg, 10.40 mmol) was added and the reaction mixture was stirred at 25° C. for 12 hours. The volatiles were removed in vacuo to afford a residue. The residue was purified by Prep-HPLC (Column: Phenomenex C18 75*30 mm*3 um, Mobile Phase A: water (NH₃H₂O+NH₄HCO₃), Mobile Phase B: acetonitrile, Flow rate: 25 mL/min, gradient condition from 17% B to 47%) and lyophilized to give the intermediate 15-Cis (310.0 mg, 32.3%-yield) as a white solid and the intermediate 15-trans (320.0 mg, 33.4% yield) as a white solid. LC-MS (ESI+): m/z 231.2 (M+H)⁺.

To a solution of intermediate 15-Cis (310.0 mg, 1.35 mmol) in MeOH (5.0 mL) was added HCl/dioxane (5.0 mL, 4 M) and the reaction mixture was stirred at 20° C. for 1 hour. The volatiles were removed in vacuo to give the intermediate 15-1 (324.0 mg, crude) as a white solid, which was used in the next step without further purification.

### Example 21

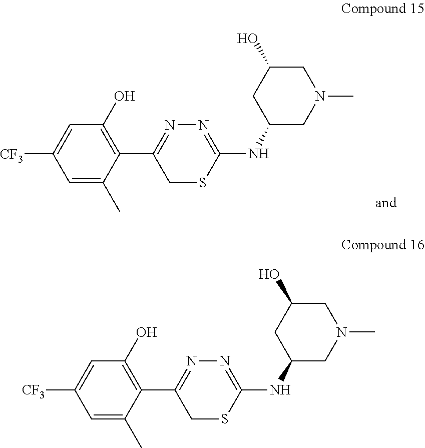 [SMILES: Cc1cc(C(F)(F)F)cc(O)c1C1=NN=C(N[C@@H]2C[C@H](O)CN(C)C2)SC1 ; Cc1cc(C(F)(F)F)cc(O)c1C1=NN=C(N[C@H]2C[C@@H](O)CN(C)C2)SC1]

In a similar fashion according to the procedure for Compound 12, Compound 15 and 16 were synthesized by replacing (1R,2R)-2-aminocyclopentan-1-ol with Intermediate 15-1. The crude product was purified by Prep-SFC (Column: DAICEL CHIRALPAK IG (250 mm*30 mm, 10 um), Mobile Phase: CO₂-EtOH (0.1% NH₃H₂O), Flow rate: 80 mL/min, gradient condition from 35% B to 35%) to give the title compound 15 and 16.

Compound 15 (the first peak): LC-MS (ESI+): m/z 403.2 (M+H)⁺. ¹H NMR (400 MHz, DMSO-d₆) δ=10.47 (br. s, 1H), 7.37-7.06 (m, 2H), 7.00 (s, 1H), 4.87-4.80 (m, 1H), 4.08-3.85 (m, 1H), 3.61-3.50 (m, 1H), 3.43-3.37 (m, 2H), 3.03-2.91 (m, 1H), 2.85-2.75 (m, 1H), 2.28 (s, 3H), 2.19 (s, 3H), 2.11-2.03 (m, 1H), 1.68-1.56 (m, 2H), 1.19-1.08 (m, 1H).

Compound 16 (the second peak): LC-MS (ESI+): m/z 403.2 (M+H)⁺. ¹H NMR (400 MHz, DMSO-d₆) δ=10.47 (br. s, 1H), 7.08 (s, 1H), 7.05-6.74 (m, 2H), 4.87-4.80 (m, 1H), 4.08-3.85 (m, 1H), 3.61-3.50 (m, 1H), 3.44-3.37 (m, 2H), 3.04-2.92 (m, 1H), 2.85-2.75 (m, 1H), 2.28 (s, 3H), 2.19 (s, 3H), 2.12-2.03 (m, 1H), 1.69-1.54 (m, 2H), 1.20-1.08 (m, 1H).

### Example 22

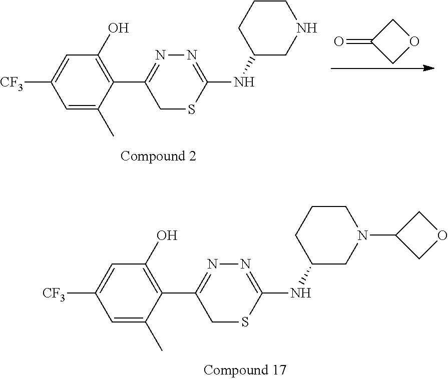

To a solution of compound 2 (150.0 mg, 0.25 mmol, 75% purity, HBr salt) in MeOH (1.5 mL) was added TEA (84 μL, 0.60 mmol) and the reaction mixture was stirred at 25° C. for 15 min. Then, oxetan-3-one (54.4 mg, 0.76 mmol) and HOAc (112.7 mg, 1.88 mmol) were added in sequence and the mixture was stirred at 25° C. for 30 min. Then NaBH₃CN (19.0 mg, 0.30 mmol) was added and the mixture was stirred at 25° C. for 2 hours. The mixture was purified by Prep-TLC (DCM:MeOH=10:1), Prep-SFC (Column: DAICEL CHIRALCEL OD (250 mm*30 mm, 10 μm), Condition: CO₂-EtOH (0.1% NH₃H₂O), Begin B: 20%, Flow Rate: 150 ml/min) and then by Prep-HPLC (Column: Phenomenex C₁₈ 80*40 mm*3 μm, Mobile Phase A: water (NH₃H₂O+NH₄HCO₃), Mobile Phase B: acetonitrile, Flow rate: 30 mL/min, gradient condition from 29% B to 59%) to give the compound 17.

LC-MS (ESI⁺): m/z 429.1 (M+H)⁺.

¹H NMR (400 MHz, DMSO-d₆) δ=10.47 (br. s, 1H), 7.08 (s, 1H), 7.04-6.82 (m, 2H), 4.56-4.49 (m, 2H), 4.47-4.40 (m, 2H), 4.08-3.91 (m, 1H), 3.44-3.41 (m, 2H), 2.89-2.79 (m, 1H), 2.60-2.52 (m, 2H), 2.30 (s, 3H), 1.93-1.67 (m, 4H), 1.59-1.46 (m, 1H), 1.37-1.28 (m, 1H).

### Example 23

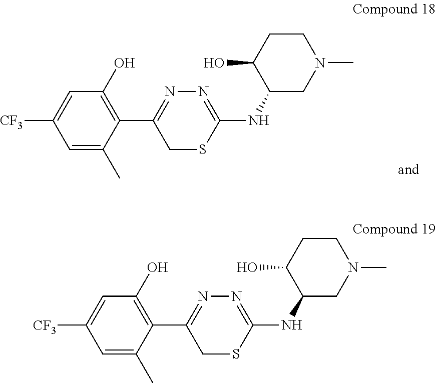 [SMILES: Cc1cc(C(F)(F)F)cc(O)c1C1=NN=C(N[C@H]2CN(C)CC[C@@H]2O)SC1 ; Cc1cc(C(F)(F)F)cc(O)c1C1=NN=C(N[C@@H]2CN(C)CC[C@H]2O)SC1]

In a similar fashion according to the procedure for Compound 12, Compound 18 and 19 were synthesized by replacing (1R,2R)-2-aminocyclopentan-1-ol with (trans)-3-amino-1-methylpiperidin-4-ol hydrochloride. The crude product was separated by Prep-SFC (Column: DAICEL CHIRALPAK AD (250 mm*30 mm, 10 μm), Mobile Phase: CO₂-EtOH (0.1% NH₃H₂O), Flow rate: 70 mL/min, gradient condition 25%) and then purified by Prep-HPLC: Welch Xtimate C18 150*25 mm*5 μm, Mobile Phase: water (NH₃H₂O+NH₄HCO₃)-ACN, Flow rate: 25 mL/min, gradient condition from 12% to 42% to give the title compound 18 and 19.

Compound 18 (the first peak): LC-MS (ESI+): m/z 403.2 (M+H)⁺. ¹H NMR (400 MHz, DMSO-d₆) δ=10.49 (br. s., 1H), 7.08 (s, 1H), 7.05-6.62 (m, 2H), 5.11-4.66 (m, 1H), 3.96-3.64 (m, 1H), 3.44-3.40 (m, 2H), 3.04-2.89 (m, 1H), 2.72-2.58 (m, 1H), 2.29 (s, 3H), 2.16 (s, 3H), 2.14-2.02 (m, 1H), 1.98-1.88 (m, 1H), 1.87-1.73 (m, 2H), 1.56-1.41 (m, 1H).

Compound 19 (the second peak): LC-MS (ESI+): m/z 403.2 (M+H)⁺. ¹H NMR (400 MHz, DMSO-d₆) δ=10.49 (br. s., 1H), 7.08 (s, 1H), 7.05-6.62 (m, 2H), 5.11-4.66 (m, 1H), 3.96-3.64 (m, 1H), 3.44-3.40 (m, 2H), 3.04-2.89 (m, 1H), 2.72-2.58 (m, 1H), 2.29 (s, 3H), 2.16 (s, 3H), 2.14-2.06 (m, 1H), 1.98-1.88 (m, 1H), 1.87-1.73 (m, 2H), 1.56-1.41 (m, 1H).

### Example 24

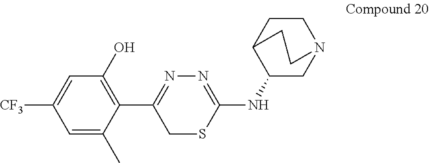 [SMILES: Cc1cc(C(F)(F)F)cc(O)c1C1=NN=C(N[C@H]2CN3CCC2CC3)SC1]

In a similar fashion according to the procedure for Compound 1, Compound 20 was synthesized by replacing intermediate 1-4 with (R)-quinuclidin-3-amine. The crude product was purified by Prep-HPLC (Column: Phenomenex C18 75*30 mm*3 μm, Mobile Phase A: water (NH₃H₂O+NH₄HCO₃), Mobile Phase B: acetonitrile, Flow rate: 25 mL/min, gradient condition from 30% B to 60%) to give the compound 20.

LC-MS (ESI⁺): m/z: 399.1 (M+H)⁺.

¹H NMR (400 MHz, DMSO-d₆) δ=7.07 (s, 1H), 7.01 (s, 1H), 4.02-3.91 (m, 1H), 3.41-3.37 (m, 2H), 3.19-3.10 (m, 1H), 2.90-2.79 (m, 1H), 2.74-2.65 (m, 3H), 2.62-2.54 (m, 1H), 2.29 (s, 3H), 2.03-1.97 (m, 1H), 1.87-1.77 (m, 1H), 1.65-1.54 (m, 2H), 1.39-1.29 (m, 1H).

### Example 25

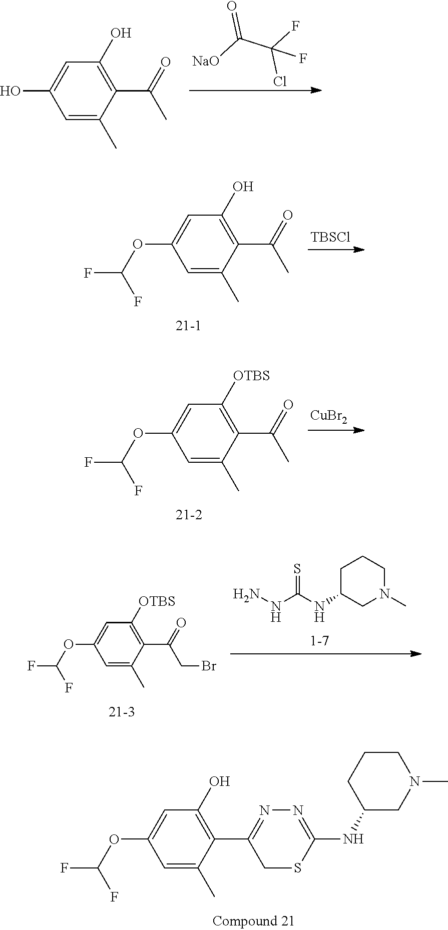

To a solution of 1-(2,4-dihydroxy-6-methylphenyl) ethan-1-one (2.40 g, 14.44 mmol) and K₂CO₃ (3.00 g, 21.71 mmol) in DMF (12.0 mL) and H₂O (5.0 mL) at 90° C. was added a solution of sodium 2-chloro-2,2-difluoroacetate (2.20 g, 14.44 mmol) in DMF (8.0 mL) at 90° C. under N₂. The resulting mixture was stirred at 90° C. for 4 hours. After cooling to room temperature, the mixture was quenched with H₂O (60 mL) and diluted with ethyl acetate (60 mL). Then, the mixture was acidified with citric acid aqueous solution (20 mL) and the aqueous phase was extracted with ethyl acetate (50 mL×3). The combined organic extracts were dried over Na₂SO₄, filtered through a celite pad and the filtrate was concentrated under reduced pressure to afford a crude. The crude was purified by flash column chromatography on silica gel eluted with 0˜20% ethyl acetate in petroleum ether to give the intermediate 21-1 (370.0 mg, 11.9% yield) as a colorless oil.

To a solution of intermediate 21-1 (370.0 mg, 1.71 mmol) in DCM (6.0 mL) were added TBSCl (567.4 mg, 3.76 mmol), DMAP (33.5 mg, 0.27 mmol) and TEA (0.71 mL, 5.13 mmol) at 20° C. and the mixture was stirred at 20° C. for 1.5 hours. H₂O (30 mL) was added and the mixture was extracted with DCM (30 mL×3). The combined organic extracts were dried over Na₂SO₄, filtered through a celite pad and the filtrate was concentrated under reduced pressure to afford a crude. The crude was purified by flash column chromatography on silica gel eluted with 0˜20% ethyl acetate in petroleum ether to give the intermediate 21-2 (520.0 mg, 98.68% purity, 90.7% yield) as a colorless oil. LC-MS (ESI+): m/z 331.2 (M+H)⁺.

To a solution of intermediate 21-2 (520.0 mg, 1.57 mmol) in DCE (3.0 mL) was added a mixture of CuBr₂ (632.7 mg, 2.83 mmol) in ethyl acetate (6.0 mL) at 80° C. and the mixture was stirred at 80° C. for 1 hour. After cooling to room temperature, the mixture was concentrated under reduced pressure to afford a residue. The residue was purified by flash column chromatography on silica gel eluted with 0˜10% ethyl acetate in petroleum ether to give the intermediate 21-3 (270.0 mg, 90.7% purity, 38.1% yield) as a colorless oil. LC-MS (ESI+): m/z 409.1 (M+H)⁺.

To a solution of intermediate 21-3 (170.0 mg, 0.42 mmol) and intermediate 1-7 (93.4 mg, 0.42 mmol) in EtOH (2.0 mL) was added conc. HCl (51.9 μL, 0.62 mmol) at 30° C. and the mixture was stirred at 80° C. for 1 hour. After cooling to room temperature, the mixture was adjusted to pH=13 with NH₃·H₂O (0.3 mL) and the volatiles were removed in vacuo to afford a residue. The residue was purified by Prep-HPLC (Column: Welch Xtimate C18 150*25 mm*5 μm, Mobile Phase A: water (10 mM NH₄HCO₃), Mobile Phase B: acetonitrile, Flow rate: 25 mL/min, gradient condition from 30% B to 60%) and lyophilized to give the compound 21.

LC-MS (ESI+): m/z: 385.2 (M+H)⁺.

¹H NMR (400 MHz, DMSO-d₆) δ=10.28 (br. s., 1H), 7.19 (t, J=74.4 Hz, 1H), 6.94-6.72 (m, 1H), 6.55-6.50 (m, 2H), 4.03-3.84 (m, 1H), 3.39-3.37 (m, 2H), 2.96-2.86 (m, 1H), 2.64-2.56 (m, 1H), 2.22 (s, 3H), 2.16 (s, 3H), 1.90-1.71 (m, 3H), 1.69-1.61 (m, 1H), 1.53-1.45 (m, 1H), 1.25-1.22 (m, 1H).

### Example 26

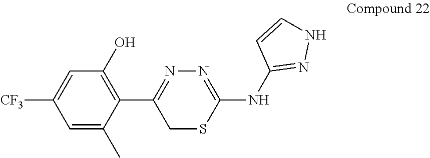 [SMILES: Cc1cc(C(F)(F)F)cc(O)c1C1=NN=C(Nc2cc[nH]n2)SC1]

In a similar fashion according to the procedure for Compound 12, Compound 22 was synthesized by replacing (1R,2R)-2-aminocyclopentan-1-ol with 1-((2-(trimethylsilyl) ethoxy)methyl)-1H-pyrazol-3-amine. The crude product was purified by Prep-HPLC (Column: Welch Xtimate C18 150*30 mm*5 μm, Mobile Phase: water (FA)-ACN, Flow rate: 25 mL/min, gradient condition from 23% B to 53%) and Prep-SFC (Column: DAICEL CHIRALPAK AD (250 mm*30 mm, 10 μm), Mobile Phase: CO₂-EtOH (0.1% NH₃H₂O), Flow rate: 80 mL/min, gradient condition from 45% B to 45%) to give the compound 22.

LC-MS (ESI⁺): m/z 356.0 (M+H)⁺.

¹H NMR (400 MHz, DMSO-d₆) δ=12.32 (br. s., 1H), 11.21 (br. s., 1H), 7.62-7.53 (m, 1H), 7.03 (s, 1H), 6.99 (s, 1H), 6.12-5.71 (m, 1H), 3.55-3.47 (m, 2H), 2.30 (s, 3H).

### Example 27

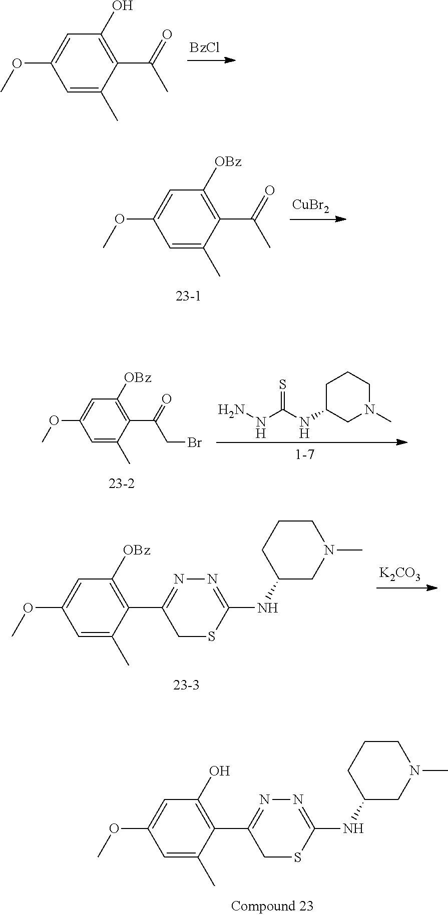

To a solution of 1-(2-hydroxy-4-methoxy-6-methylphenyl) ethanone (1.00 g, 5.55 mmol) and DMAP (135.6 mg, 1.11 mmol) in DCM (30.0 mL) was added TEA (0.77 mL, 5.55 mmol) at 25° C. Then benzoyl chloride (0.97 mL, 8.32 mmol) was added to the mixture at 0° C. dropwise. The resulting mixture was stirred at 25° C. for 1 hour. The mixture was diluted with water (20 mL) and the aqueous phase was extracted with dichloromethane (20 mL×3). The combined organic layers were dried over anhydrous Na₂SO₄, filtered through a celite pad and the filtrate was concentrated under reduced pressure to give a crude. The crude was purified by flash column chromatography on silica gel eluted with 0˜100% ethyl acetate in petroleum ether to give the intermediate 23-1 (1.50 g, 95.1% yield) as colorless oil.

To a solution of intermediate 23-1 (500.0 mg, 1.76 mmol) in DCE (5.0 mL) and EA (5.0 mL) was added CuBr₂ (706.9 mg, 3.16 mmol) at 20° C. and the resulting mixture was stirred at 80° C. for 3 hours. After cooling to room temperature, the volatiles were removed in vacuo to give a residue. The residue was purified by flash column chromatography on silica gel eluted with 0˜50% dichloromethane in petroleum ether to give the intermediate 23-2 (500.0 mg, 78.2% yield) as colorless oil.

To a solution of intermediate 1-7 (154.7 mg, 0.69 mmol) and acetic acid (0.08 mL, 1.40 mmol) in EtOH (4.0 mL) was added intermediate 23-2 (250.0 mg, 0.69 mmol) at 20° C. Then, the resulting mixture was heated to 60° C. and stirred for 2 hours. After cooling to room temperature, the mixture was adjusted to pH=13 with NH₃·H₂O (0.3 mL) and concentrated under reduced pressure to afford a residue. The residue was purified by Prep-HPLC (Column: Welch Xtimate C18 150*25 mm*5 μm, Mobile Phase A: water (NH₃H₂O+NH₄HCO₃), Mobile Phase B: acetonitrile, Flow rate: 25 mL/min, gradient condition from 30% to 60% B) and lyophilized to give the intermediate 23-3 (50.0 mg, 94.9% purity, 15.2% yield) as yellow solid. LC-MS (ESI⁺): m/z: 453.4 (M+H)⁺.

To a solution of intermediate 23-3 (45.0 mg, 0.10 mmol) in MeOH (2.0 mL) was added K₂CO₃ (41.2 mg, 0.30 mmol) at 20° C. and the mixture was heated to 50° C. and stirred for 1 hour. After cooling to room temperature, the volatiles were removed in vacuo to afford a residue. The residue was purified by Prep-HPLC (Column: Welch Xtimate C18 150*25 mm*5 μm, Mobile Phase A: water (NH₃H₂O+NH₄HCO₃), Mobile Phase B: acetonitrile, Flow rate: 25 mL/min, gradient condition from 18% to 48% B) to give the compound 23.

LC-MS (ESI⁺): m/z: 349.1 (M+H)⁺.

¹H NMR (400 MHz, DMSO-d₆) δ=10.13 (br. s., 1H), 6.31-6.27 (m, 2H), 3.98-3.87 (m, 1H), 3.70 (s, 3H), 3.40-3.36 (m, 2H), 2.96-2.87 (m, 1H), 2.64-2.57 (m, 1H), 2.21 (s, 3H), 2.17 (s, 3H), 1.92-1.73 (m, 3H), 1.70-1.62 (m, 1H), 1.55-1.43 (m, 1H), 1.28-1.15 (m, 1H).

### Example 28

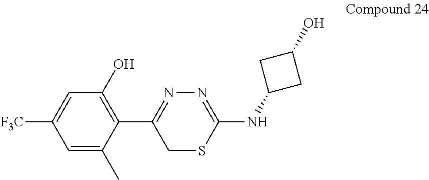 [SMILES: Cc1cc(C(F)(F)F)cc(O)c1C1=NN=C(N[C@H]2C[C@@H](O)C2)SC1]

In a similar fashion according to the procedure for Compound 12, Compound 24 was synthesized by replacing (1R,2R)-2-aminocyclopentan-1-ol with (1s,3s)-3-aminocyclobutan-1-ol. The crude product was purified by Prep-HPLC (Column: Phenomenex C18 75*30 mm*3 μm, Mobile Phase: water (NH₃H₂O+NH₄HCO₃)-ACN, Flow rate: 25 mL/min, gradient condition from 27% B to 57%) to give the compound 24.

LC-MS (ESI+): m/z 360.0 (M+H)⁺.

¹H NMR (400 MHz, DMSO-d₆) δ=10.38 (br. s., 1H), 7.07 (s, 1H), 7.00 (s, 1H), 5.14-5.04 (m, 1H), 3.94-3.76 (m, 2H), 3.40-3.35 (m, 2H), 2.63-2.53 (m, 2H), 2.28 (s, 3H), 1.89-1.76 (m, 2H).

### Example 29

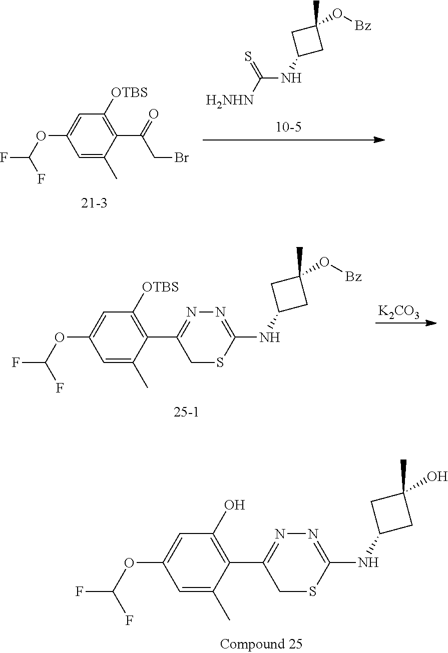

To a solution of intermediate 21-3 (160.0 mg, 0.39 mmol) and intermediate 10-5 (218.4 mg, 0.78 mmol) in EtOH (4.0 mL) was added HOAc (0.14 mL, 2.42 mmol) at 30° C. and the mixture was heated to 60° C. and stirred for 4 hours. After cooling to room temperature, the mixture was concentrated under reduced pressure to afford a residue. The residue was adjusted to pH=8 with saturated NaHCO₃ aqueous solution and then extracted with ethyl acetate (20 mL×3). The combined organic extracts were dried over Na₂SO₄, filtered through a celite pad and the filtrate was concentrated under reduced pressure to afford a crude. The crude was purified by flash column chromatography on silica gel eluted with 0˜100% ethyl acetate in petroleum ether to give the intermediate 25-1 (50.0 mg, 94.07% purity, 25.3% yield) as a yellow oil. LC-MS (ESI+): m/z 476.3 (M+H)⁺.

To a solution of intermediate 25-1 (35.0 mg, 0.074 mmol) in MeOH (2.0 mL) was added K₂CO₃ (30.5 mg, 0.22 mmol) at 20° C. and the mixture was stirred at 35° C. for 7 hours. The volatiles were removed in vacuo to afford a residue. The residue was purified by Prep-HPLC (Column: Phenomenex C18 75*30 mm*3 μm, Mobile Phase A: water (0.05% NH₃H₂O+10 mM NH₄HCO₃), Mobile Phase B: acetonitrile, Flow rate: 25 mL/min, gradient condition from 25% B to 55%) and lyophilized to give the compound 25.

LC-MS (ESI⁺): m/z 372.1 (M+H)⁺.

¹H NMR (400 MHz, DMSO-d₆) δ=10.33 (br. s., 1H), 7.19 (t, J=74.0 Hz, 1H), 6.55-6.53 (m, 1H), 6.53-6.50 (m, 1H), 4.97 (s, 1H), 3.99-3.86 (m, 1H), 3.39-3.35 (m, 2H), 2.35-2.27 (m, 2H), 2.22 (s, 3H), 2.05-1.96 (m, 2H), 1.25 (s, 3H).

### Example 30

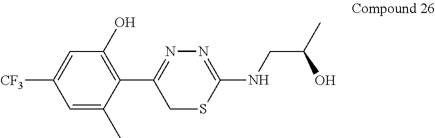 [SMILES: Cc1cc(C(F)(F)F)cc(O)c1C1=NN=C(NC[C@@H](C)O)SC1]

In a similar fashion according to the procedure for Compound 12, Compound 26 was synthesized by replacing (1R,2R)-2-aminocyclopentan-1-ol with (R)-2-((tert-butyldiphenylsilyl)oxy)propan-1-amine. In the last step, the reaction mixture was stirred at −78° C. for 8 hours. The crude product was purified by prep-HPLC (Column: Welch Xtimate C18 150*25 mm*5 μm, Mobile Phase A: water (NH₃·H₂O+NH₄HCO₃), Mobile Phase B: acetonitrile, Flow rate: 25 mL/min, gradient condition from 37% B to 67%) and lyophilized to give the compound 26.

LC-MS (ESI+): m/z: 347.9 (M+H)⁺.

¹H NMR (400 MHz, DMSO-d₆) δ=10.46 (br. s., 1H), 7.10-7.04 (m, 1H), 7.04-6.98 (m, 1H), 4.92-4.81 (m, 1H), 3.90-3.78 (m, 1H), 3.39-3.36 (m, 2H), 3.28-3.25 (m, 2H), 2.29 (s, 3H), 1.08 (d, J=5.6 Hz, 3H).

### Example 31

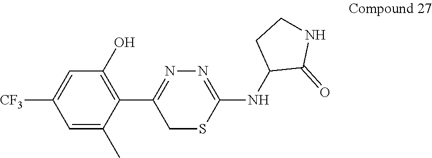 [SMILES: Cc1cc(C(F)(F)F)cc(O)c1C1=NN=C(NC2CCNC2=O)SC1]

In a similar fashion according to the procedure for Compound 12, Compound 27 was synthesized by replacing (1R,2R)-2-aminocyclopentan-1-ol with 3-aminopyrrolidin-2-one. The crude product was purified by Prep-HPLC (Column: Welch Xtimate C18 150*25 mm*5 um, Mobile Phase A: water (NH₃H₂O+NH₄HCO₃), Mobile Phase B: acetonitrile, Flow rate: 25 mL/min, gradient condition from 38% B to 68%) to give the compound 27.

LC-MS (ESI+): m/z: 373.1 (M+H)⁺.

¹H NMR (400 MHz, DMSO-d₆) δ=10.83-10.13 (m, 1H), 7.86 (s, 1H), 7.08 (s, 1H), 7.02 (s, 1H), 4.74-4.21 (m, 1H), 3.48-3.42 (m, 2H), 3.25-3.19 (m, 2H), 2.47-2.38 (m, 1H), 2.30 (s, 3H), 2.01-1.88 (m, 1H).

### Example 32

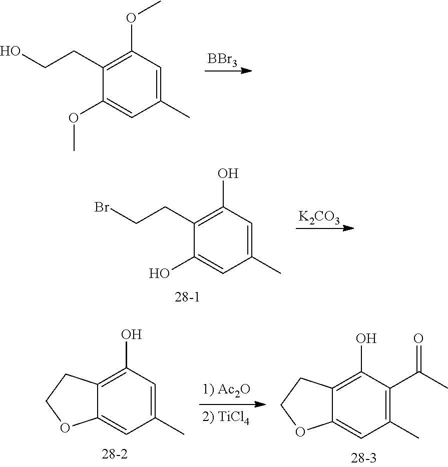

To a solution of 2-(2,6-dimethoxy-4-methylphenyl) ethan-1-ol (5.00 g, 25.48 mmol, which was prepared according to the reference: JACS, 2004, 126, 11966-11983.) in DCM (100 mL) was added BBr₃ (5.40 mL, 56.07 mmol) dropwise at 0° C. and the mixture was stirred at room temperature for 1 hour. The reaction mixture was quenched by adding MeOH (15 mL) at 0° C. and the volatiles were removed in vacuo to give a residue. H₂O (20 mL) was added and the aqueous phase was extracted with DCM (20 mL×3). The combined organic extracts were washed with brine (20 mL), dried over anhydrous Na₂SO₄, filtered through a celite pad and the filtrate was concentrated under reduced pressure to give the intermediate 28-1 (5.0 g, crude) as brown oil.

To a solution of intermediate 28-1 (5.0 g, crude) in acetone (400 mL) was added K₂CO₃ (15.00 g, 108.54 mmol) and the mixture was stirred at 70° C. for 1 hour. The reaction mixture was concentrated under reduced pressure to afford a residue. The residue was purified by flash column chromatography on silica gel eluted with 20% ethyl acetate in petroleum ether to give the intermediate 28-2 (2.74 g, 84% purity, 60.2% yield over 2 steps) as a white solid.

LC-MS (ESI+): m/z: 151.1 (M+H)⁺.

¹H NMR (400 MHz, DMSO-d₆) δ=9.25 (br. s, 1H) 6.10 (s, 1H) 6.05 (s, 1H) 4.44 (t, J=8.80 Hz, 2H) 2.96 (t, J=8.80 Hz, 2H) 2.13 (s, 3H).

To a solution of intermediate 28-2 (2.74 g, 18.24 mmol) in DCM (50.0 mL) were added acetic anhydride (5.64 mL, 60.20 mmol) and a solution of TiCl₄ (13.00 mL, 118.58 mmol) in DCM (25.0 mL) at 0° C. in sequence. The mixture was stirred at 0° C. for 1 hour and at 25° C. for another 5 hours. The mixture was quenched with H₂O (20 mL) at 0° C., diluted with DCM (90 mL) and washed with H₂O (30 mL×2) and brine (30 mL), dried over anhydrous Na₂SO₄, filtered through a celite pad and the filtrate was concentrated under reduced pressure to afford a residue. The residue was purified by flash column chromatography on silica gel eluted with 20% ethyl acetate in petroleum ether to give the intermediate 28-3 (2.22 g, 63.3% yield) as a white solid.

LC-MS (ESI+): m/z: 193.1 (M+H)⁺.

¹H NMR (400 MHz, DMSO-d₆) δ=10.77 (br. s, 1H) 6.21 (s, 1H) 4.54 (t, J=8.80 Hz, 2H) 3.06 (t, J=8.80 Hz, 2H) 2.47 (s, 3H) 2.22 (s, 3H).

### Example 33

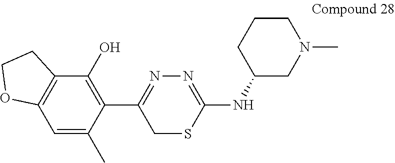 [SMILES: Cc1cc2c(c(O)c1C1=NN=C(N[C@@H]3CCCN(C)C3)SC1)CCO2]

In a similar fashion according to the procedure for Compound 23, Compound 28 was synthesized by replacing 1-(2-hydroxy-4-methoxy-6-methylphenyl) ethan-1-one with intermediate 28-3. The crude product was purified by Prep-HPLC (Column: Phenomenex C18 75*30 mm*3 um, Mobile Phase A: water (NH₃H₂O+NH₄HCO₃), Mobile Phase B: acetonitrile, Flow rate: 20 mL/min, gradient condition from 40% B to 70%) and lyophilized to give the compound 28.

LC-MS (ESI+): m/z 361.2 (M+H)⁺.

¹H NMR (400 MHz, CD₃CN) δ=6.26 (s, 1H), 4.58 (t, J=8.80 Hz, 2H), 4.14-4.03 (m, 1H), 3.55 (s, 2H), 3.10 (t, J=8.80 Hz, 2H), 2.85-2.71 (m, 1H), 2.52-2.41 (m, 1H), 2.34 (s, 3H), 2.22 (s, 3H), 2.12-2.08 (m, 2H), 1.83-1.70 (m, 2H), 1.62-1.53 (m, 1H), 1.51-1.43 (m, 1H).

### Example 34

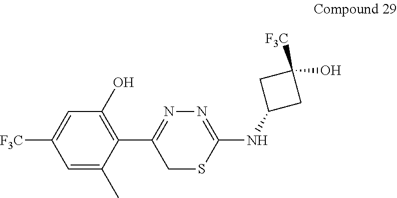 [SMILES: Cc1cc(C(F)(F)F)cc(O)c1C1=NN=C(N[C@H]2C[C@](O)(C(F)(F)F)C2)SC1]

In a similar fashion according to the procedure for Compound 12, Compound 29 was synthesized by replacing (1R,2R)-2-aminocyclopentan-1-ol with (1s,3s)-3-amino-1-(trifluoromethyl)cyclobutan-1-ol. The crude product was purified by Prep-HPLC (Column: C18 150×30 mm, Mobile Phase A: water (NH₃H₂O+NH₄HCO₃), Mobile Phase B: acetonitrile, Flow rate: 25 mL/min, gradient condition from 42% B to 72%) and lyophilized to give the compound 29.

LC-MS (ESI+): m/z 428.1 (M+H)⁺.

¹H NMR (400 MHz, DMSO-d₆) δ=10.45 (br. s., 1H), 7.52 (br. s., 1H), 7.08 (s, 1H), 7.00 (s, 1H), 6.63 (s, 1H), 4.21-4.00 (m, 1H), 3.50-3.40 (m, 2H), 2.85-2.73 (m, 2H), 2.28 (s, 3H), 2.27-2.21 (m, 2H).

### Example 35

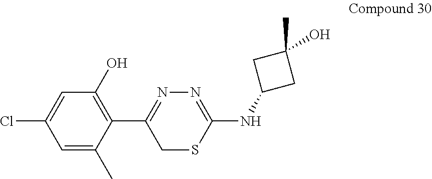 [SMILES: Cc1cc(Cl)cc(O)c1C1=NN=C(N[C@H]2C[C@@](C)(O)C2)SC1]

In a similar fashion according to the procedure for Compound 25, Compound 30 was synthesized by replacing intermediate 21-1 with 1-(4-chloro-2-hydroxy-6-methylphenyl) ethan-1-one. The crude product was purified by Prep-HPLC (Column: Xtimate C18 150*40 mm*10 μm, Mobile Phase A: water (FA), Mobile Phase B: acetonitrile, Flow rate: 30 mL/min, gradient condition from 12% B to 42%) and lyophilized to give the compound 30.

LC-MS (ESI+): m/z 340.1 (M+H)⁺.

¹H NMR (400 MHz, DMSO-d₆) δ=10.67 (br. s, 1H), 8.14 (s, 1H from HCOOH), 6.79 (d, J=1.6 Hz, 1H), 6.76 (d, J=1.6 Hz, 1H), 4.96 (s, 1H), 4.01-3.86 (m, 1H), 3.39-3.35 (m, 2H), 2.34-2.26 (m, 2H), 2.20 (s, 3H), 2.04-1.96 (m, 2H), 1.24 (s, 3H).

### Example 36

Intermediate 37-4 was used to synthesize compound 37. Similarly, other intermediates (from example 37 to example 54) were used to synthesize corresponding compounds

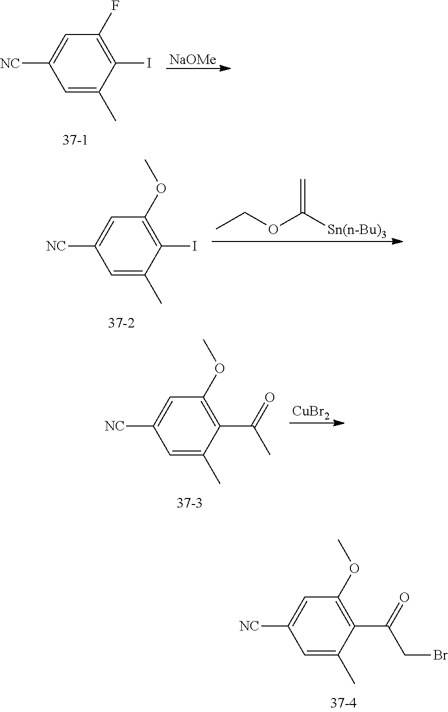

To a mixture of compound 37-1 (5.00 g, 19.16 mmol) in DMF (50.0 mL) was added NaOMe (40 mL, 30% in MeOH) at 25° C. and the mixture was stirred at 50° C. for 1 hour. Cold H₂O (60 mL) was added and the mixture was extracted with ethyl acetate (50 mL×3). The combined organic extracts were dried over Na₂SO₄, filtered through a celite pad and the filtrate was concentrated under reduced pressure to afford a crude. The crude was purified by flash column chromatography on silica gel eluted with 0˜10% ethyl acetate in petroleum ether to give the intermediate 37-2 (1.30 g, 90.0% purity, 22.4% yield) as white solid.

LC-MS (ESI+): m/z: 274.0 (M+H)⁺.

¹H NMR (400 MHz, CDCl₃) δ 7.16 (d, J=1.2 Hz, 1H), 6.83 (d, J=1.2 Hz, 1H), 3.93 (s, 3H), 2.51 (s, 3H).

To a mixture of intermediate 37-2 (200.0 mg, 0.73 mmol), CuI (14.0 mg, 0.074 mmol) and TEA (0.20 mL, 1.47 mmol) in dioxane (4.0 mL) was added tributyl(1-ethoxyvinyl)stannane (100.0 mg, 0.28 mmol). Then Pd(PPh₃)₂Cl₂ (84.6 mg, 0.12 mmol) was added under N₂ and the mixture was stirred at 100° C. for 12 hours under N₂. After cooling to room temperature, HCl (10 mL, 1 M) was added and the mixture was stirred for 1 h. The reaction mixture was quenched with saturated KF aqueous solution (40 mL) and extracted with ethyl acetate (50 mL×2). The combined organic extracts were washed with brine (50 mL), dried over Na₂SO₄, filtered through a celite pad and the filtrate was concentrated under reduced pressure to afford a crude. The crude was purified by flash column chromatography on silica gel eluted with 0˜10% ethyl acetate in petroleum ether to give the intermediate 37-3 (90.0 mg, 86.5% purity, 56.2% yield) as yellow oil.

LC-MS (ESI+): m/z: 190.1 (M+H)⁺

A solution of CuBr₂ (191.2 mg, 0.86 mmol) in EtOAc (1.0 mL) was added dropwise to a mixture of intermediate 37-3 (90.0 mg, 0.48 mmol) in DCE (1.0 mL) at 80° C. After the addition, the mixture was stirred at 80° C. for 3 hours. The reaction mixture was concentrated under reduced pressure and purified by flash column chromatography on silica gel eluted with 0˜10% ethyl acetate in petroleum ether to give the intermediate 37-4 (80.0 mg, 75.0% purity, 46.6% yield) as white solid.

LC-MS (ESI+): m/z: 268.0 (M+H)⁺.

### Example 37

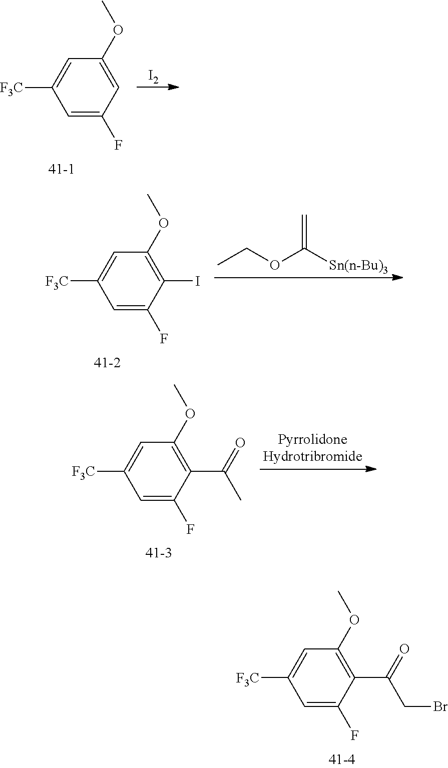

To a solution of compound 41-1 (5.00 g, 25.76 mmol) in THF (30 mL) was added n-BuLi (10.3 mL, 25.76 mmol, 2.5M in hexane) at −78° C. under N₂. After stirred at −78° C. for 1 h, a solution of I₂ (7.19 g, 28.34 mmol) in THF (25 mL) was added and the mixture was stirred at −78° C. for another 2 h. To the reaction mixture was added water (100 mL) and extracted with EtOAc (50 mL×3). The combined organic layers were washed with brine (200 mL), dried over Na₂SO₄, filtered through a celite pad and the filtrate was concentrated under reduced pressure to give a residue, which was purified by column chromatography on silica gel eluted with petroleum ether to give the intermediate 41-2 (4.00 g, 48.5% yield) as yellow oil.

¹H NMR (400 MHz, CDCl₃) δ=7.03-6.97 (m, 1H), 6.82 (s, 1H), 3.97 (s, 3H).

To a solution of intermediate 41-2 (3.50 g, 10.94 mmol) in toluene (25 mL) were added tributyl(1-ethoxyvinyl)stannane (5.53 g, 15.31 mmol) and Pd(PPh₃)₄ (253.0 mg, 0.22 mmol). The reaction was stirred at 110° C. for 16 h under N₂. To the cooled reaction mixture was added 6 N HCl (15 mL) and stirred at room temperature for 2 h. To the reaction mixture was added water (10 mL) and the mixture was extracted with EtOAc (40 mL×2). The combined organic layers were washed with brine (40 mL×3), dried over Na₂SO₄, filtered through a celite pad and the filtrate was concentrated under reduced pressure to give a residue, which was purified by column chromatography on silica gel eluted with ethyl acetate (from 0% to 8%) in petroleum ether to give the intermediate 41-3 (2.50 g, 96.8% yield) as yellow oil.

¹H NMR (400 MHz, CDCl₃) δ=7.05-6.96 (m, 1H), 6.96 (s, 1H), 3.91 (s, 3H), 2.55 (s, 3H). To a solution of intermediate 41-3 (2.50 g, 10.59 mmol) in THF (15 mL) was added pyrrolidone hydrotribromide (4.13 g, 12.70 mmol). The reaction was stirred at 40° C. for 3 h. The reaction mixture was added water (10 mL) and extracted with EtOAc (15 mL×2). The combined organic layers were washed with brine (30 mL), dried over Na₂SO₄, filtered through a celite pad and the filtrate was concentrated under reduced pressure to give a residue, which was purified by column chromatography on silica gel eluted with 0˜7% ethyl acetate in petroleum ether to give the intermediate 41-4 (2.40 g, 72.0% yield) as yellow oil.

¹H NMR (400 MHz, CDCl₃) δ=7.12-7.02 (m, 2H), 4.34 (s, 2H), 3.95 (s, 3H).

### Example 38

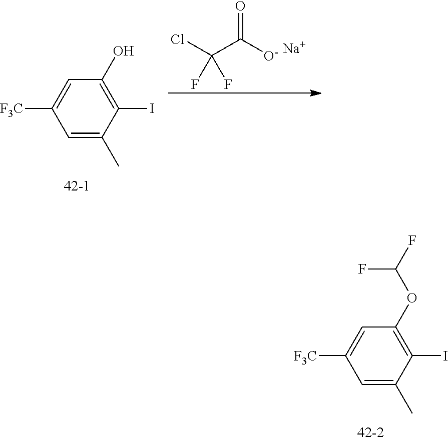 [SMILES: Cc1cc(C(F)(F)F)cc(O)c1I ; O=C([O-])C(F)(F)Cl ; [Na+] ; Cc1cc(C(F)(F)F)cc(OC(F)F)c1I]

To a solution of compound 42-1 (1.60 g, 5.30 mmol) in DMF (10 mL) were added sodium chlorodifluoroacetate (2.00 g, 13.11 mmol) and Cs₂CO₃ (3.50 g, 10.74 mmol). The reaction was stirred at 100° C. for 2 h. To the cooled reaction mixture was added water (20 mL) and the mixture was extracted with EtOAc (20 mL×2). The combined organic layers were washed with brine (20 mL), dried over Na₂SO₄, filtered through a celite pad and the filtrate was concentrated under reduced pressure to give a residue, which was purified by column chromatography on silica gel eluted with petroleum ether to give the intermediate 42-2 (0.80 g, 42.9% yield) as colorless oil.

¹H NMR (400 MHz, CDCl₃) δ=7.37 (s, 1H), 7.19 (s, 1H), 6.57 (t, J=72.8 Hz, 1H), 2.57 (s, 3H).

### Example 39

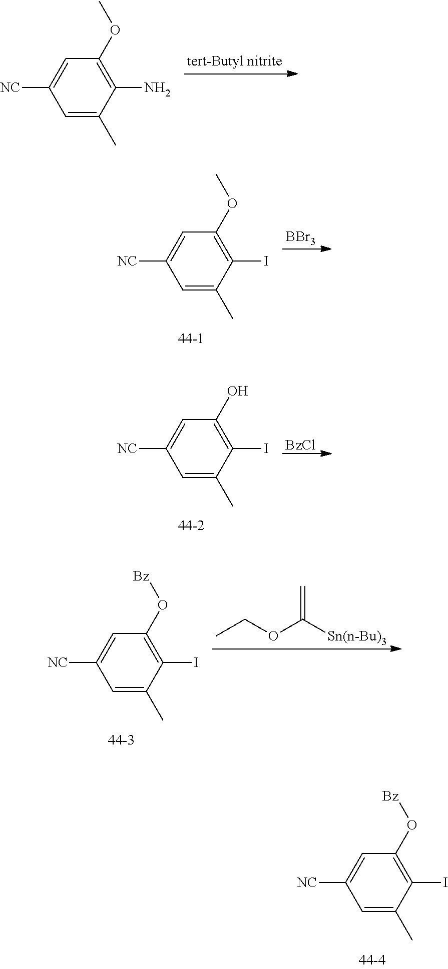

To a solution of 4-amino-3-methoxy-5-methylbenzene-1-carbonitrile (3.50 g, 21.58 mmol) in ACN (50 mL) were added CuI (6.16 g, 32.37 mmol) and tert-butyl nitrite (4.45 g, 43.16 mmol). The reaction was stirred at room temperature for 16 h. The mixture was concentrated under reduced pressure to give a residue, which was purified by column chromatography on silica gel eluted with 0˜8% ethyl acetate in petroleum ether to give the intermediate 44-1 (4.10 g, 69.6% yield) as white solid.

¹H NMR (400 MHz, CDCl₃) δ=7.15 (d, J=1.2 Hz, 1H), 6.82 (d, J=1.2 Hz, 1H), 3.91 (s, 3H), 2.50 (s, 3H).

To a solution of intermediate 44-1 (2.50 g, 9.15 mmol) in DCM (80 mL) was added tribromoborane (45.8 mL, 45.80 mmol, 1M in DCM) at 0° C. The mixture was stirred at 50° C. for 32 h. The mixture was quenched with MeOH at 0° C. dropwise and then the mixture was concentrated under reduced pressure to give a residue, which was purified by column chromatography on silica gel eluted with 0˜8% ethyl acetate in petroleum ether to give the intermediate 44-2 (1.10 g, 46.4% yield) as white solid.

¹H NMR (400 MHz, CDCl₃) δ=7.08-7.06 (m, 2H), 5.83 (s, 1H), 2.49 (s, 3H).

To a solution of intermediate 44-2 (1.10 g, 4.25 mmol) and TEA (0.43 g, 4.25 mmol) in DCM (10 mL) were added 4-(dimethylamino)pyridine (0.095 g, 0.85 mmol) and benzoyl chloride (0.90 g, 6.37 mmol). The reaction was stirred at rt for 1 h. The reaction mixture was concentrated under reduced pressure to give a residue, which was purified by column chromatography on silica gel eluted with 0˜8% ethyl acetate in petroleum ether to give the intermediate 44-3 (1.50 g, 97.2% yield) as white solid.

¹H NMR (400 MHz, CDCl₃) δ=8.28-8.26 (m, 2H), 7.72-7.68 (m, 1H), 7.66-7.56 (m, 2H), 7.42 (d, J=1.2 Hz, 1H), 7.33 (d, J=1.2 Hz, 1H), 2.57 (s, 3H).

To a solution of intermediate 44-3 (1.50 g, 4.13 mmol) in toluene (15 mL) were added tributyl(1-ethoxyvinyl)stannane (2.09 g, 5.78 mmol) and Pd(PPh₃)₄ (0.1 g, 0.087 mmol). The reaction was stirred at 120° C. for 16 h under Ar. To the cooled reaction mixture was added 1 M HCl (10 mL) and the reaction was stirred at room temperature for another 4 h. To the reaction mixture was added water (40 mL) and the mixture was extracted with EtOAc (40 mL×2). The combined organic layers were washed with brine (80 mL), dried over Na₂SO₄, filtered through a celite pad and the filtrate was concentrated under reduced pressure to give a residue, which was purified by column chromatography on silica gel eluted with 0˜8% ethyl acetate in petroleum ether to give the intermediate 44-4 (897.0 mg, 77.7% yield) as yellow oil.

¹H NMR (400 MHz, CDCl₃) δ=8.06-8.04 (m, 2H), 7.65-7.57 (m, 1H), 7.48-7.37 (m, 4H), 2.40 (s, 3H), 2.29 (s, 3H).

### Example 40

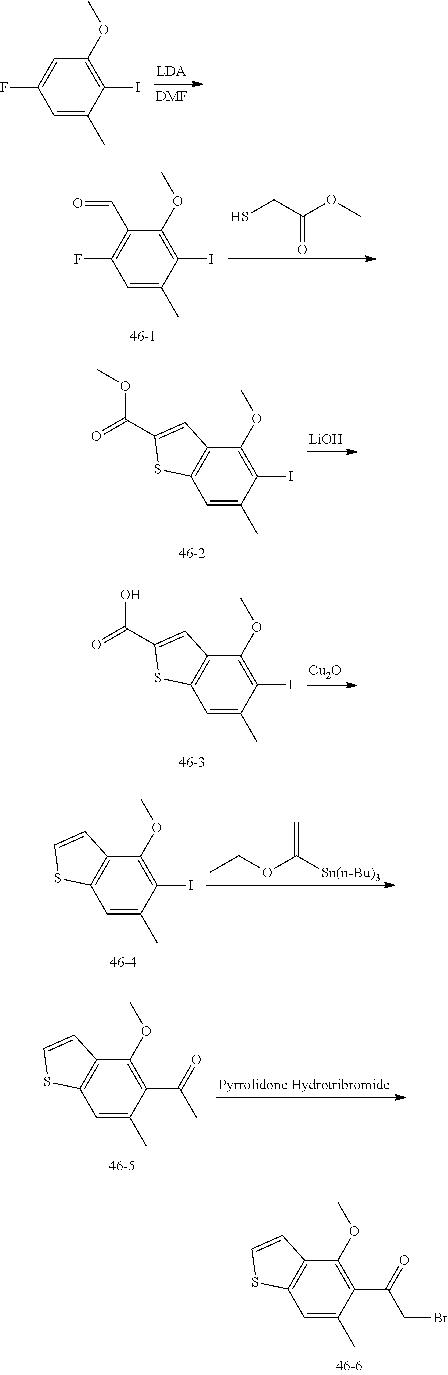

To a solution of 5-fluoro-2-iodo-1-methoxy-3-methylbenzene (3.90 g, 14.66 mmol) in THF (30 mL) was added Lithium diisopropylamide (8.8 mL, 17.59 mmol, 2M in THF) dropwise at −78° C. under N₂. After stirred at −78° C. for 1 h, dry DMF (1.6 mL, 20.52 mmol) was added dropwise to the mixture and the reaction mixture was stirred at −78° C. for 45 minutes. The reaction was quenched with HCl (30 mL, 1 M) at 20° C. The mixture was extracted with EtOAc (40 mL×2). The combined organic layers were washed with brine (40 mL×2), dried over Na₂SO₄, filtered through a celite pad and the filtrate was concentrated under reduced pressure to give a residue, which was purified by column chromatography on silica gel eluted with 0˜9% ethyl acetate in petroleum ether to give the intermediate 46-1 (3.10 g, 71.9% yield) as yellow solid.

¹H NMR (400 MHz, CDCl₃) δ=10.27 (s, 1H), 6.95 (d, J=10.8 Hz, 1H), 3.91 (s, 3H), 2.53 (s, 3H).

To a solution of intermediate 46-1 (3.36 g, 11.43 mmol) in DMF (20 mL) were added methyl 2-mercaptoacetate (1.82 g, 17.14 mmol) and K₂CO₃ (4.74 g, 34.27 mmol). The mixture was stirred at 80° C. for 1 h. To the cooled reaction mixture was added water (20 mL) and the mixture was extracted with EtOAc (30 mL×2). The combined organic layers were washed with brine (30 mL×2), dried over Na₂SO₄, filtered through a celite pad and the filtrate was concentrated under reduced pressure to give a residue, which was purified by column chromatography on silica gel eluted with 0˜9% ethyl acetate in petroleum ether to give the intermediate 46-2 (2.20 g, 53.1% yield) as yellow solid.

¹H NMR (400 MHz, CDCl₃) δ=8.11 (s, 1H), 7.53 (s, 1H), 4.00 (s, 3H), 3.95 (s, 3H), 2.60 (s, 3H).

To a solution of intermediate 46-2 (2.20 g, 6.07 mmol) in THF/H₂O (15 mL/5 mL) was added Lithium hydroxide (0.73 g, 30.37 mmol). The reaction was stirred at room temperature for 16 h. To the reaction mixture was added water (10 mL) and the pH was adjusted to 4 by adding 4N HCl (10 mL). The mixture was extracted with EtOAc (40 mL×2). The combined organic layers were washed with brine (40 mL×2), dried over Na₂SO₄, filtered through a celite pad and the filtrate was concentrated under reduced pressure to give the intermediate 46-3 (2.00 g, 94.6% yield) as yellow solid, which was used for next step directly.

LC-MS (ESI−): m/z 346.9 (M−H).

To a solution of intermediate 46-3 (1.00 g, 2.87 mmol) in DMF (10 mL) was added Cu₂O (1.64 g, 11.49 mmol). The reaction was stirred at 140° C. for 16 h under N₂. After cooling to room temperature, the mixture was filtered through a celite pad, and the filtrate was added to water (15 mL) and the mixture was extracted with EtOAc (30 mL×2). The combined organic layers were washed with brine (30 mL×2), dried over Na₂SO₄, filtered and concentrated under reduced pressure to give a residue, which was purified by column chromatography on silica gel eluted with 0˜5% ethyl acetate in petroleum ether to give the intermediate 46-4 (0.60 g, 68.7% yield) as yellow oil.

¹H NMR (400 MHz, DMSO-d₆) δ=7.77 (s, 1H), 7.72 (d, J=5.2 Hz, 1H), 7.48-7.46 (m, 1H), 3.89 (s, 3H), 2.52 (s, 3H).

To a solution of intermediate 46-4 (400.0 mg, 1.32 mmol) in toluene (10 mL) were added tributyl(1-ethoxyvinyl)stannane (665.0 mg, 1.84 mmol) and Pd(PPh₃)₄ (30.0 mg, 0.026 mmol). The reaction was stirred at 120° C. for 16 h under N₂. After cooling to room temperature, to the reaction mixture was added 6 N HCl (10 mL) and the mixture was stirred at room temperature for 2 h. To the reaction mixture was added water (10 mL) and the mixture was extracted with EtOAc (20 mL×3). The combined organic layers were washed with brine (20 mL×3), dried over Na₂SO₄, filtered and concentrated under reduced pressure to give a residue, which was purified by column chromatography on silica gel eluted with 0˜10% ethyl acetate in petroleum ether to give the intermediate 46-5 (280.0 mg, 96.2% yield) as yellow oil.

¹H NMR (400 MHz, CDCl₃) δ=7.46 (s, 1H), 7.42-7.37 (m, 2H), 3.95 (s, 3H), 2.58 (s, 3H), 2.36 (s, 3H).

To a solution of intermediate 46-5 (380.0 mg, 1.73 mmol) in THF (10 mL) was added pyrrolidone hydrotribromide (1121.0 mg, 3.45 mmol). The reaction was stirred at 40° C. for 4 h. The reaction mixture was concentrated under reduced pressure to give a residue, which was purified by column chromatography on silica gel eluted with 0˜5% ethyl acetate in petroleum ether to give the intermediate 46-6 (210.0 mg, 40.5% yield) as yellow oil.

¹H NMR (400 MHz, DMSO-d₆) δ=7.78 (d, J=5.6 Hz, 1H), 7.69 (s, 1H), 7.59-7.58 (m, 1H), 4.71 (s, 2H), 3.97 (s, 3H), 2.29 (s, 3H).

### Example 41

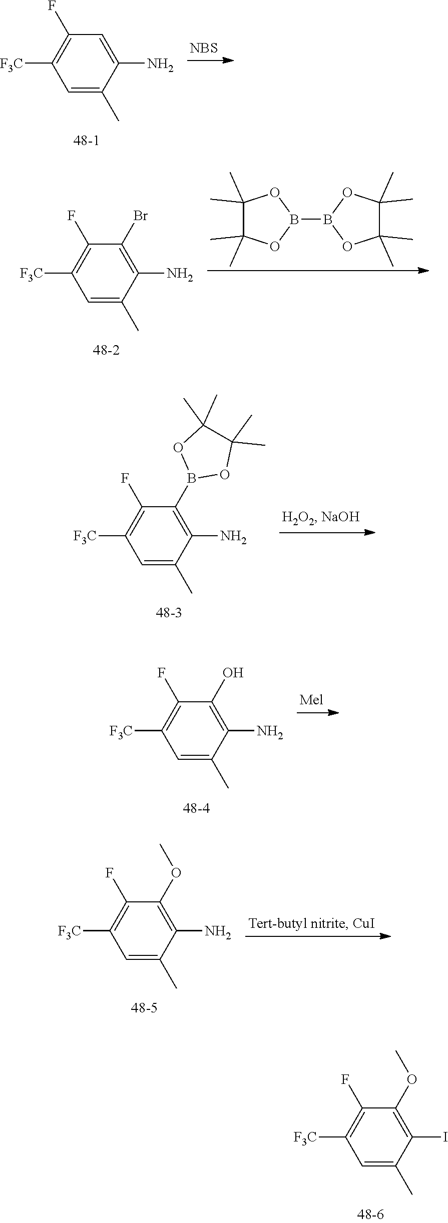

To a solution of compound 48-1 (3.80 g, 19.68 mmol) in ACN (40 mL) was added a solution of NBS (3.87 g, 21.76 mmol) in ACN (30 mL) at 0° C. The mixture was stirred at room temperature for 2 h. To the mixture was added water (80 mL) and the mixture was extracted with EtOAc (80 mL×2). The combined organic layers were washed with brine (80 mL), dried over Na₂SO₄, filtered and the filtrate was concentrated under reduced pressure to give a residue, which was purified by column chromatography on silica gel eluted with 0˜5% ethyl acetate in petroleum ether to give the intermediate 48-2 (4.30 g, 80.3% yield) as yellow oil.

¹H NMR (400 MHz, CDCl₃) δ=7.19 (d, J=7.6 Hz, 1H), 4.51 (br.s, 2H), 2.20 (s, 3H).

To a solution of intermediate 48-2 (4.30 g, 15.81 mmol) in dioxane (80 mL) were added 4,4,5,5-tetramethyl-2-(4,4,5,5-tetramethyl-1,3,2-dioxaborolan-2-yl)-1,3,2-dioxaborolane (8.03 g, 31.62 mmol), Pd(dppf)Cl₂·CH₂Cl₂ (1.28 g, 1.58 mmol) and KOAc (3.88 g, 39.52 mmol). The mixture was stirred at 100° C. for 16 hr under N₂. To the cooled mixture was added water (80 mL) and the mixture was extracted with EtOAc (80 mL×2). The combined organic layers were washed with brine (80 mL), dried over Na₂SO₄, filtered and the filtrate was concentrated under reduced pressure to give a residue, which was purified by column chromatography on silica gel eluted with 0˜5% ethyl acetate in petroleum ether to give the intermediate 48-3 (4.20 g, crude) as yellow oil.

¹H NMR (400 MHz, CDCl₃) δ=7.26-7.15 (m, 1H), 4.60-4.47 (m, 2H), 2.09 (s, 3H), 1.37 (s, 12H).

To a solution of intermediate 48-3 (4.20 g, crude) in THF (50 mL) were slowly added NaOH (1.58 g, 39.49 mmol, 2N) and H₂O₂ (8.95 g, 78.97 mmol, 30% in H₂O) at 0° C. The mixture was stirred at rt for 2 h. To the mixture was added water (50 mL) and the mixture was extracted with EtOAc (60 mL×2). The combined organic layers were washed with brine (60 mL), dried over Na₂SO₄, filtered and concentrated under reduced pressure to give a residue, which was purified by column chromatography on silica gel eluted with 0˜5% ethyl acetate in petroleum ether to give the intermediate 48-4 (0.85 g, 25.7% yield over two steps) as yellow oil.

LC-MS(ESI+): m/z 210.1 (M+H)⁺.

¹H NMR (400 MHz, CDCl₃) δ=6.85 (d, J=7.6 Hz, 1H), 5.18 (br.s, 1H), 4.07 (br.s, 2H), 2.15 (s, 3H).

A mixture of intermediate 48-4 (0.85 g, 4.06 mmol) and K₂CO₃ (843.0 mg, 6.10 mmol) in DMF (12 mL) was stirred at 0° C. for 0.5 h. To the mixture was slowly added CH₃I (634.0 mg, 4.47 mmol) and the mixture was stirred at 0° C. for 1.5 h. To the mixture was added water (50 mL) and extracted with EtOAc (60 mL×2). The combined organic layers were washed with brine (50 mL×2), dried over Na₂SO₄, filtered and the filtrate was concentrated under reduced pressure to give a residue, which was purified by column chromatography on silica gel eluted with 0˜5% ethyl acetate in petroleum ether to give the intermediate 48-5 (0.50 g, 55.2% yield) as yellow oil.

LC-MS(ESI+): m/z 224.1 (M+H)⁺.

¹H NMR (400 MHz, CDCl₃) δ=6.96 (d, J=7.2 Hz, 1H), 4.16 (br.s, 2H), 3.94 (d, J=1.6 Hz, 3H), 2.13 (s, 3H).

To a solution of intermediate 48-5 (400.0 mg, 1.79 mmol) in ACN (12 mL) were added tert-butyl nitrite (369.2 mg, 3.58 mmol) and CuI (512.0 mg, 2.69 mmol). The reaction was stirred at 60° C. for 4 h. After cooling to room temperature, the mixture was concentrated under reduced pressure to give a residue, which was purified by column chromatography on silica gel eluted with petroleum ether to give the intermediate 48-6 (400.0 mg, 67.0% yield) as colorless oil.

¹H NMR (400 MHz, CDCl₃) δ=7.27-7.16 (m, 1H), 3.97 (d, J=1.6 Hz, 3H), 2.48 (s, 3H).

### Example 42

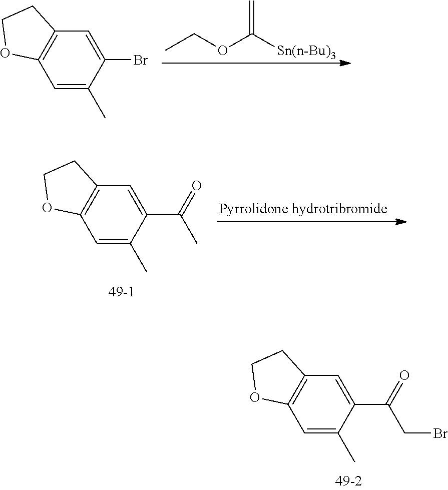

To a solution of 5-bromo-6-methyl-2,3-dihydrobenzofuran (130.0 mg, 0.61 mmol) in toluene (4 mL) were added tributyl(1-ethoxyvinyl)-stannane (308.0 mg, 0.85 mmol) and Pd(PPh₃)₄ (13.0 mg, 0.011 mmol). The reaction was stirred at 120° C. for 16 h under N₂. After cooling to room temperature, to the reaction mixture was added 6 N HCl (3 mL, 18.00 mmol). The mixture was stirred at room temperature for 2 h. To the reaction mixture was added water (10 mL) and the mixture was extracted with EtOAc (10 mL×3). The combined organic layers were washed with brine (10 mL), dried over Na₂SO₄, filtered and concentrated under reduced pressure to give a residue, which was purified by column chromatography on silica gel eluted with 0˜5% ethyl acetate in petroleum ether to give the intermediate 49-1 (80.0 mg, 74.4% yield) as white solid.

¹H NMR (400 MHz, CDCl₃) δ=7.64 (s, 1H), 6.64 (s, 1H), 4.63 (t, J=8.4 Hz, 2H), 3.22 (t, J=8.4 Hz, 2H), 2.54 (s, 3H), 2.53 (s, 3H).

To a solution of intermediate 49-1 (80.0 mg, 0.45 mmol) in THF (4 mL) was added Pyrrolidone hydrotribromide (146.7 mg, 0.45 mmol). The reaction was stirred at 40° C. for 4 h. To the cooled reaction mixture was added water (10 mL) and the mixture was extracted with EtOAc (10 mL×3). The combined organic layers were washed with brine (10 mL), dried over Na₂SO₄, filtered and the filtrate was concentrated under reduced pressure to give the intermediate 49-2 (110.0 mg, 95.6%) as yellow oil, which was used for next step without further workup.

¹H NMR (400 MHz, CDCl₃) δ=7.63 (s, 1H), 6.69 (s, 1H), 4.66 (t, J=8.8 Hz, 2H), 4.37 (s, 2H), 3.24 (t, J=8.4 Hz, 2H), 2.53 (s, 3H).

### Example 43

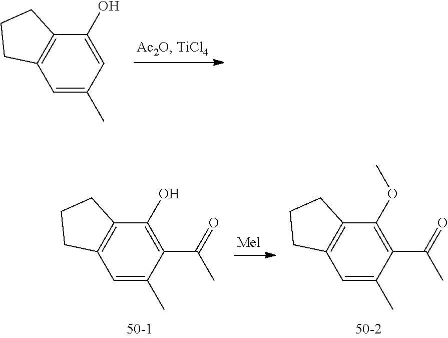

To a solution of 6-methyl-2,3-dihydro-1H-inden-4-ol (2.40 g, 16.19 mmol) in DCM (50 mL) were added acetic anhydride (5.46 g, 53.44 mmol) and TiCl₄ (19.66 g, 103.64 mmol) in DCM (30 mL) at 0° C. The reaction was stirred at 0° C. for 1 h and then stirred at room temperature for 2 h. The mixture was quenched by water (15 mL) at 0° C. and the mixture was extracted with DCM (60 mL×2). The combined organic layers were washed with brine (50 mL), dried over Na₂SO₄, filtered and the filtrate was concentrated under reduced pressure to give a residue, which was purified by column chromatography on silica gel eluted with 0˜3% ethyl acetate in petroleum ether to give the intermediate 50-1 (2.3 g, crude) as yellow oil, which was used for next step directly.

LC-MS(ESI⁺): m/z 191.2 (M+H)⁺.

To a mixture of intermediate 50-1 (2.30 g, crude) and K₂CO₃ (2.51 g, 18.14 mmol) in DMF (30 mL) was added CH₃I (2.23 g, 15.72 mmol). The mixture was stirred at room temperature for 1.5 h. To the mixture was added water (30 mL) and the mixture was extracted with EtOAc (30 mL×2). The combined organic layers were washed with brine (40 mL×2), dried over Na₂SO₄, filtered and concentrated under reduced pressure to give a residue, which was purified by column chromatography on silica gel eluted with 0˜3% ethyl acetate in petroleum ether to give the intermediate 50-2 (1.00 g, 30.2% yield over 2 steps) as yellow solid.

LC-MS(ESI+): m/z 205.2 (M+H)⁺.

¹H NMR (400 MHz, CDCl₃) δ=6.82 (s, 1H), 3.79 (s, 3H), 2.95 (t, J=7.2 Hz, 2H), 2.86 (t, J=7.2 Hz, 2H), 2.49 (s, 3H), 2.22 (s, 3H), 2.13-2.06 (m, 2H).

### Example 44

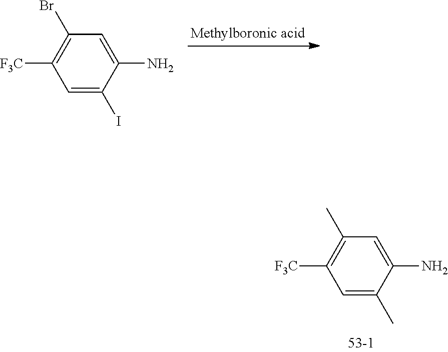

To a solution of 5-bromo-2-iodo-4-(trifluoromethyl) aniline (22.20 g, 60.67 mmol) in DMF (200 mL) were added methyl boronic acid (3.83 g, 63.95 mmol), Pd(OAc)₂ (0.34 g, 1.52 mmol), RuPhos (1.41 g, 3.03 mmol) and K₂CO₃ (50.3 g, 364.03 mmol). The mixture was degassed with nitrogen for 2 min and heated to 110° C. and stirred for 16 h. To the cooled reaction mixture was added water (200 mL) and the mixture was extracted with EtOAc (100 mL×2). The combined organic layers were washed with brine (200 mL), dried over Na₂SO₄, filtered and concentrated under reduced pressure to give a residue, which was purified by column chromatography on silica gel eluted with 0˜20% ethyl acetate in petroleum ether to give the intermediate 53-1 (6.00 g, 52.3% yield) as light yellow oil.

LC-MS(ESI+): m/z 190.1 (M+H)⁺.

### Example 45

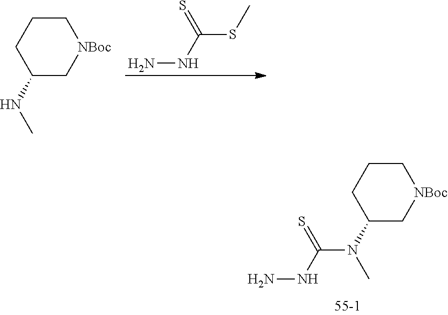

To a solution of tert-butyl-(R)-3-(methylamino) piperidine-1-carboxylate (2.00 g, 9.33 mmol) in EtOH (20 mL) was added methyl hydrazinecarbodithioate (0.57 g, 4.67 mmol). The reaction was stirred at 90° C. for 16 h. After cooling to room temperature, the mixture was concentrated under reduced pressure to give a residue, which was purified by column chromatography on silica gel eluted with 0˜5% MeOH in DCM to give the intermediate 55-1 (650.0 mg, 24.1% yield) as yellow oil.

LC-MS(ESI+): m/z 289.2 (M+H)⁺.

### Example 46

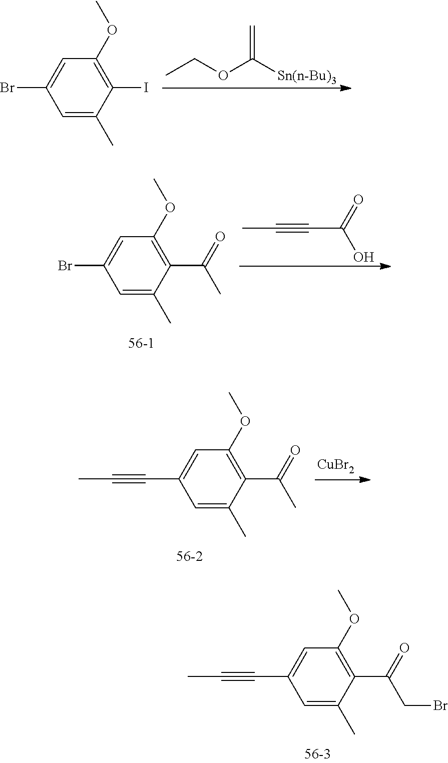

To a solution of 5-bromo-2-iodo-1-methoxy-3-methylbenzene (2.90 g, 8.87 mmol) in toluene (50 mL) were added tributyl(1-ethoxyvinyl)stannane (3.52 g, 9.76 mmol) and Pd(PPh₃)₄ (0.21 g, 0.18 mmol) under N₂. The reaction was stirred at 120° C. for 16 h under N₂. To the cooled reaction mixture was added HCl (68.0 mL, 6 mol/L) and the mixture was stirred at room temperature for 2 h. To the reaction mixture was added water (30 mL) and the mixture was extracted with EtOAc (30 mL×2). The combined organic layers were washed with brine (50 mL), dried over Na₂SO₄, filtered and concentrated under reduced pressure to give a residue, which was purified by column chromatography on silica gel eluted with 0˜3% ethyl acetate in petroleum ether to give the intermediate 56-1 (1.18 g, 54.7% yield) as yellow solid.

¹H NMR (400 MHz, CDCl₃) δ=6.98 (s, 1H), 6.90 (s, 1H), 3.82 (s, 3H), 2.46 (s, 3H), 2.21 (s, 3H).

To a solution of intermediate 56-1 (1.20 g, 4.94 mmol) and but-2-ynoic acid (623.0 mg, 7.41 mmol) in DMSO (15 mL) were added DBU (1.50 g, 9.87 mmol), 1,4-Bis (diphenylphosphino) butane (42.2 mg, 0.099 mmol) and PdCl₂(PPh₃)₂ (35.8 mg, 0.049 mmol) under N₂. The mixture was stirred at 100° C. for 4 h. To the cooled mixture was added water (60 mL) and the mixture was extracted with EtOAc (60 mL×2). The combined organic layers were washed with brine (60 mL×2), dried over Na₂SO₄, filtered and concentrated under reduced pressure to give a residue, which was purified by column chromatography on silica gel eluted with 0˜3% ethyl acetate in petroleum ether to give the intermediate 56-2 (850.0 mg, 85.1% yield) as yellow solid.

LC-MS(ESI+): m/z 203.2 (M+H)⁺.

¹H NMR (400 MHz, CDCl₃) δ=6.85 (s, 1H), 6.77 (s, 1H), 3.80 (s, 3H), 2.46 (s, 3H), 2.19 (s, 3H), 2.05 (s, 3H).

To a solution of intermediate 56-2 (850.0 mg, 4.20 mmol) in EA/DCE (5 mL/5 mL) was added CuBr₂ (1314.0 mg, 5.88 mmol). The reaction was stirred at 80° C. for 16 h. The reaction was filtered and the filtrate was concentrated under reduced pressure to give a residue, which was purified by column chromatography on silica gel eluted with 0˜3% ethyl acetate in petroleum ether to give the intermediate 56-3 (550.0 mg, 46.7% yield) as yellow solid.

LC-MS(ESI+): m/z 281.0 (M+H)⁺.

### Example 47

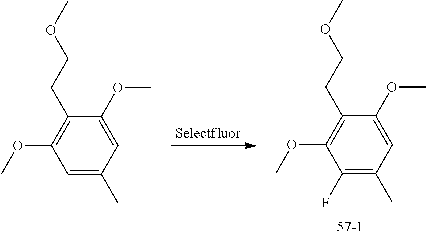

To a solution of 1,3-dimethoxy-2-(2-methoxyethyl)-5-methylbenzene (2.60 g, 12.36 mmol) in MeCN (60 mL) was added Selectfluor (3.94 g, 11.13 mmol) at 0° C. The mixture was stirred at 0° C. for 2 h. To the mixture was added water (50 mL) and the mixture was extracted with EtOAc (50 mL×2). The combined organic layers were washed with brine (100 mL), dried over Na₂SO₄, filtered and concentrated under reduced pressure to give a residue, which was purified by column chromatography on silica gel eluted with petroleum ether to give the intermediate 57-1 (1.70 g, 60% yield) as light yellow oil.

LC-MS(ESI⁺): m/z 229.1 (M+1)⁺.

### Example 48

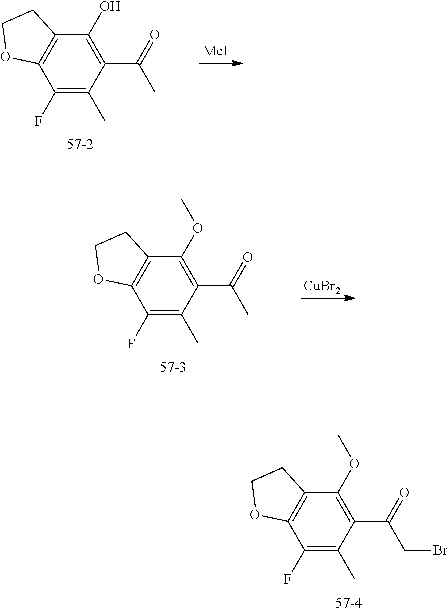

In a similar fashion according to the procedure for intermediate 28-3, intermediate 57-2 was synthesized by replacing 2-(2,6-dimethoxy-4-methylphenyl) ethan-1-ol with intermediate 57-1. To a solution of intermediate 57-2 (500.0 mg, 2.38 mmol) in DMF (10 mL) were added K₂CO₃ (657.8 mg, 4.76 mmol) and iodomethane (675.0 mg, 4.76 mmol). The mixture was stirred at room temperature for 16 h. To the reaction mixture was added water (50 mL) and the mixture was extracted with EtOAc (40 mL×2). The combined organic layers were washed with brine (80 mL), dried over Na₂SO₄, filtered and the filtrate was concentrated under reduced pressure to give a residue, which was purified by column chromatography on silica gel eluted with 0˜6% ethyl acetate in petroleum ether to give the intermediate 57-3 (510.0 mg, 95.4%) as white solid.

LC-MS(ESI+): m/z 225.1 (M+H)⁺.

¹H NMR (400 MHz, CDCl₃) δ=4.69 (t, J=8.8 Hz, 2H), 3.80 (s, 3H), 3.34 (t, J=8.8 Hz, 2H), 2.47 (s, 3H), 2.13 (d, J=2.4 Hz, 3H).

To a solution of intermediate 57-3 (450.0 mg, 2.00 mmol) in DCE/EA (10 mL/10 mL) was added CuBr₂ (583.0 mg, 2.61 mmol). The reaction was stirred at 80° C. for 16 h. The reaction mixture was filtered and the filtrate was concentrated under reduced pressure to give a residue, which was purified by prep-TLC (PE/EtOAc=5/1) to give the intermediate 57-4 (430.0 mg, 71.0% yield) as white solid.

LC-MS(ESI+): m/z 303.0 (M+H)⁺.

¹H NMR (400 MHz, CDCl₃) δ=4.72 (t, J=8.8 Hz, 2H), 4.32 (s, 2H), 3.84 (s, 3H), 3.38 (t, J=8.4 Hz, 2H), 2.17 (t, J=2.4 Hz, 3H).

### Example 49

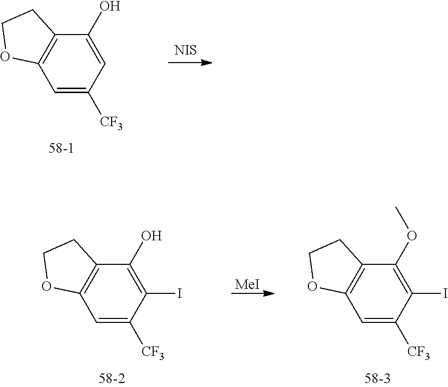 [SMILES: "MeI" ; Oc1cc(C(F)(F)F)cc2c1CCO2 ; "NIS" ; Oc1c(I)c(C(F)(F)F)cc2c1CCO2 ; COc1c(I)c(C(F)(F)F)cc2c1CCO2]

In a similar fashion according to the procedure for intermediate 28-3, intermediate 58-1 was synthesized by replacing 1,3-dimethoxy-5-methylbenzene with 1,3-dimethoxy-5-(trifluoromethyl)benzene.

To a solution of intermediate 58-1 (2.80 g, 13.72 mmol) in toluene (30 ml) was added NIS (2.46 g, 10.97 mmol) at 0° C. The reaction was stirred at room temperature for 16 h. To the reaction mixture was added water (40 mL) and extracted with EtOAc (40 mL×3). The combined organic layers were washed with saturated Na₂S₂O₃ aqueous solution (40 mL×2), dried over Na₂SO₄, filtered and the filtrate was concentrated under reduced pressure to give the intermediate 58-2 (4.20 g, crude) as yellow oil, which was used for next step directly.

LC-MS(ESI−): m/z 328.9 (M−H)⁻

To a solution of intermediate 58-2 (4.20 g, crude) in DMF (30 ml) were added iodomethane (2.17 g, 15.27 mmol)) and potassium carbonate (2.64 g, 19.09 mmol). The reaction was stirred at room temperature for 2 h. To the reaction mixture was added water (40 mL) and extracted with EtOAc (40 mL×3). The combined organic layers were washed with brine (30 mL×2), dried over Na₂SO₄, filtered and the filtrate was concentrated under reduced pressure to give a residue, which was purified by column chromatography on silica gel eluted with ethyl acetate (from 0% to 10%) in petroleum ether to give the intermediate 58-3 (1.90 g, 40.2% yield over 2 steps) as white solid.

¹H NMR (400 MHz, CDCl₃) δ=6.92 (s, 1H), 4.66 (t, J=8.4 Hz, 2H), 3.89 (s, 3H), 3.38 (t, J=8.8 Hz, 2H).

### Example 50

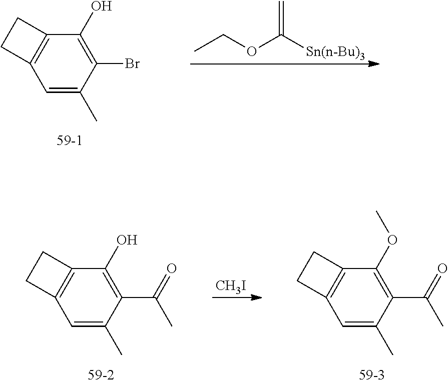

To a solution of compound 59-1 (990.0 mg, 4.65 mmol) in toluene (10 mL) were added tributyl(1-ethoxyvinyl)stannane (3356.0 mg, 9.29 mmol) and Pd(PPh₃)₄ (107.0 mg, 0.093 mmol). The reaction was stirred at 120° C. for 16 h under Ar. To the cooled reaction was added HCl (10 mL) and the mixture was stirred at room temperature for 2 h. To the reaction mixture was added water (30 mL) and the mixture was extracted with EtOAc (30 mL×2). The combined organic layers were washed with brine (50 mL), dried over Na₂SO₄, filtered and the filtrate was concentrated under reduced pressure to give a residue, which was purified by column chromatography on silica gel eluted with ethyl acetate (from 0% to 7%) in petroleum ether to give the intermediate 59-2 (500.0 mg, 61.0% yield) as yellow oil.

¹H NMR (400 MHz, CDCl₃) δ=12.20 (s, 1H), 6.53 (s, 1H), 3.26-3.13 (m, 2H), 3.12-3.02 (m, 2H), 2.63 (s, 3H), 2.57 (s, 3H).

To a solution of intermediate 59-2 (528.0 mg, 3.00 mmol) in DMF (8 mL) were added iodomethane (851 mg, 5.99 mmol) and K₂CO₃ (1.24 g, 8.99 mmol). The mixture was stirred at room temperature for 1 h. To the reaction mixture was added water (20 mL) and the mixture was extracted with EtOAc (20 mL×2). The combined organic layers were washed with brine (40 mL), dried over Na₂SO₄, filtered and the filtrate was concentrated under reduced pressure to give a residue, which was purified by column chromatography on silica gel eluted with ethyl acetate (from 0% to 7%) in petroleum ether to give the intermediate 59-3 (340.0 mg, 59.7% yield) as yellow solid.

¹H NMR (400 MHz, CDCl₃) δ=6.52 (s, 1H), 3.90 (s, 3H), 3.39 (t, J=4.0 Hz, 2H), 3.14 (t, J=4.4 Hz, 2H), 2.45 (s, 3H), 2.19 (s, 3H).

### Example 51

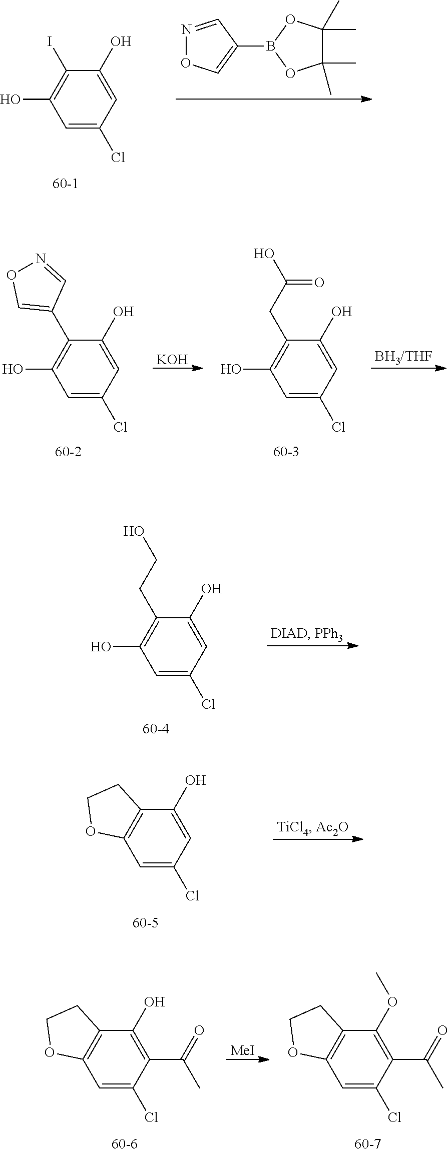

To a solution of compound 60-1 (10.50 g, 38.82 mmol), 4-(4,4,5,5-tetramethyl-1,3,2-dioxaborolan-2-yl) isoxazole (11.35 g, 58.20 mmol) and potassium fluoride (4.50 g, 77.45 mmol) in 1,4-Dioxane/Water (100 mL/20 mL) was added Pd(dppf)Cl₂ (2.84 g, 3.88 mmol). The mixture was stirred at 110° C. for 16 h under Ar. To the cooled reaction mixture was added water (300 mL) and extracted with EtOAc (300 mL×2). The combined organic layers were washed with brine (200 mL), dried over Na₂SO₄, filtered and the filtrate was concentrated under reduced pressure to give a residue, which was purified by column chromatography on silica gel eluted with ethyl acetate (from 0% to 65%) in petroleum ether to give the intermediate 60-2 (1.10 g, 13.4% yield) as yellow solid.

LC-MS (ESI+): m/z 212.0 (M+H)⁺

To a solution of intermediate 60-2 (1.10 g, 5.20 mmol) in EtOH/Water (5 mL/5 mL) was added KOH (1.50 g, 26.74 mmol). The mixture was stirred at 110° C. for 16 h under Ar. To the cooled reaction mixture was added water (20 mL). The mixture was adjusted to pH=2 with 1N HCl. Then, the mixture was extracted with EtOAc (50 mL×3). The combined organic layers were washed with brine (100 mL), dried over Na₂SO₄, filtered and the filtrate was concentrated under reduced pressure to give a residue, which was purified by reverse phase chromatography eluted with ACN (from 0% to 20%) in H₂O to give the intermediate 60-3 (400.0 mg, 38.0% yield) as yellow solid.

LC-MS (ESI): m/z 201.0 (M−H)⁻

To a solution of intermediate 60-3 (400.0 mg, 1.97 mmol) in THF (5 mL) was added BH₃ (5.92 mL, 5.92 mmol, 1M in THF) at 0° C. under Ar. The reaction was stirred at room temperature for 2 h. To the reaction mixture was added water (50 mL). The mixture was adjusted to pH=3 with 1N HCl. Then, the mixture was extracted with EtOAc (50 mL×2). The combined organic layers were washed with brine (30 mL), dried over Na₂SO₄, filtered and the filtrate was concentrated under reduced pressure to give a residue, which was purified by column chromatography on silica gel eluted with ethyl acetate (from 0% to 60%) in petroleum ether to give the intermediate 60-4 (370.0 mg, 99.6% yield) as colorless oil.

¹H NMR (400 MHz, CDCl₃) δ=6.86 (s, 2H), 6.47 (s, 2H) 3.95 (t, J=4.8 Hz, 2H), 2.93 (t, J=4.8 Hz, 2H), 2.58 (s, 1H).

To a solution of intermediate 60-4 (370.0 mg, 1.96 mmol) in THF (5 mL) were added triphenylphosphine (772.0 mg, 2.94 mmol) and DIAD (0.57 mL, 2.94 mmol) at 0° C. under Ar. The reaction was stirred at room temperature for 16 h. The reaction mixture was concentrated under reduced pressure to give a residue, which was purified by column chromatography on silica gel eluted with ethyl acetate (from 0% to 15%) in petroleum ether to give the intermediate 60-5 (200.0 mg, 59.8% yield) as yellow solid.

¹H NMR (400 MHz, CDCl₃) δ=6.42 (s, 1H), 6.34 (s, 1H), 4.99 (s, 1H), 4.64-4.59 (m, 2H), 3.13-3.09 (m, 2H).

To a solution of intermediate 60-5 (200.0 mg, 1.17 mmol) in DCM (5 mL) were added Ac₂O (395.09 mg, 3.87 mmol) and a solution of TiCl₄ (0.84 mL, 7.66 mmol) in DCM (5 mL) at 0° C. The reaction was stirred at 0° C. for 1 h. Then the reaction was warmed to room temperature and stirred for 3 h. To the reaction mixture was added water (20 mL) and extracted with EtOAc (20 mL×2). The combined organic layers were washed with brine (20 mL), dried over Na₂SO₄, filtered and the filtrate was concentrated under reduced pressure to give a residue, which was purified by column chromatography on silica gel eluted with ethyl acetate (from 0% to 5%) in petroleum ether to give the intermediate 60-6 (200.0 mg, 80.4% yield) as white solid.

LC-MS (ESI+): m/z 213.1 (M+H)⁺

¹H NMR (400 MHz, CDCl₃) δ=13.38 (s, 1H), 6.50 (s, 1H), 4.70 (t, J=8.8 Hz, 2H), 3.17 (t, J=8.8 Hz, 2H), 2.80 (s, 3H).

To a solution of intermediate 60-6 (100.0 mg, 0.47 mmol) in DMF (5 mL) were added iodomethane (79.5 mg, 0.56 mmol) and K₂CO₃ (97.0 mg, 0.70 mmol). The reaction was stirred at room temperature for 2 h. To the reaction mixture was added water (10 mL) and extracted with EtOAc (10 mL×3). The combined organic layers were washed with brine (10 mL×3), dried over Na₂SO₄, filtered and the filtrate was concentrated under reduced pressure to give a residue, which was purified by column chromatography on silica gel eluted with ethyl acetate (from 0% to 10%) in petroleum ether to give the intermediate 60-7 (100.0 mg, 93.6% yield) as white solid.

¹H NMR (400 MHz, CDCl₃) δ=6.55 (s, 1H), 4.61 (t, J=8.8 Hz, 2H), 3.86 (s, 3H), 3.32 (t, J=8.8 Hz, 2H), 2.49 (s, 3H).

### Example 52

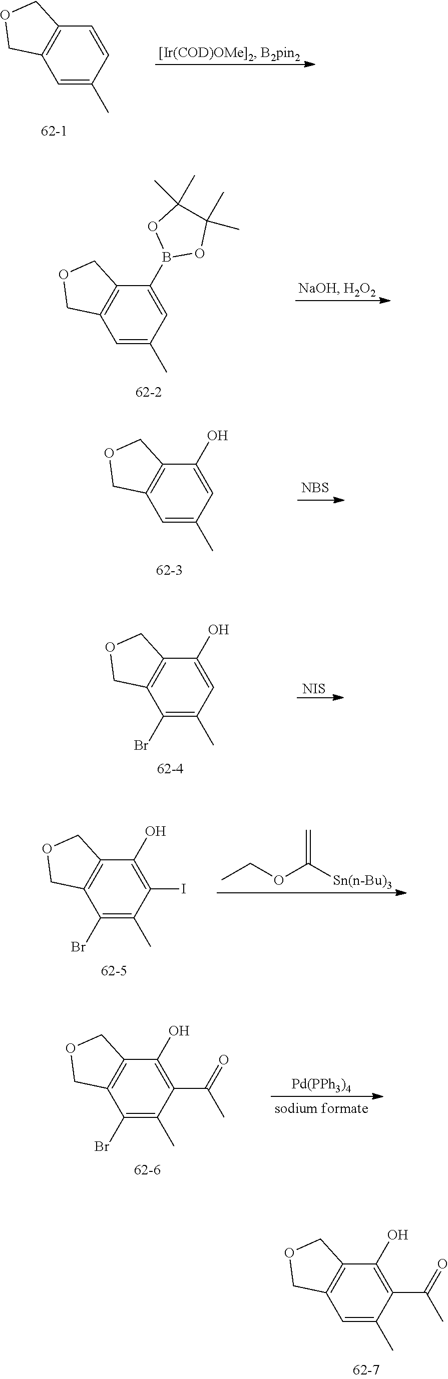

To a solution of compound 62-1 (6.10 g, 45.45 mmol) in THF (80 mL) were added 4,4,4′,4′,5,5,5′,5′-octamethyl-2,2′-bi(1,3,2-dioxaborolane) (12.70 g, 50.00 mmol), 4,4′-di-tert-butyl-2,2′-bipyridine (0.24 g, 0.91 mmol) and [Ir(COD)OMe]₂ (0.30 g, 0.46 mmol). The reaction was stirred at 70° C. for 2 h under N₂. The reaction mixture was concentrated under reduced pressure to give the intermediate 62-2 (11.00 g, crude) as brown oil, which was used for next step without further purification.

LC-MS(ESI+): m/z 261.1 (M+H)⁺

To a solution of intermediate 62-2 (11.00 g, crude) in THF (90 mL) were slowly added NaOH (42.5 mL, 85.00 mmol, 2 mol/L in H₂O) and H₂O₂ (28.80 g, 254.12 mmol, 30 wt % in H₂O) at 0° C. The mixture was stirred at room temperature for 2 h. To the mixture was added water (90 mL) and extracted with EtOAc (90 mL×3). The combined organic layers were washed with saturated sodium sulfite aqueous solution (60 mL×2), dried over Na₂SO₄, filtered and the filtrate was concentrated under reduced pressure to give a residue, which was purified by column chromatography on silica gel eluted with ethyl acetate (from 0% to 20%) in petroleum ether to give the intermediate 62-3 (1.40 g, 20.5% yield over 2 steps) as off-white solid.

LC-MS(ESI+): m/z 151.2 (M+H)⁺.

¹H NMR (400 MHz, CDCl₃) δ=6.62 (s, 1H), 6.47 (s, 1H), 5.33 (br.s, 1H), 5.11 (s, 2H), 5.08 (s, 2H), 2.31 (s, 3H).

To a solution of intermediate 62-3 (700.0 mg, 4.66 mmol) in HOAc (15 mL) was added NBS (830.0 mg, 4.66 mmol) at 0° C. The reaction was stirred at rt for 1 h. To the reaction mixture was added water (30 mL) and extracted with EtOAc (30 mL×2). The combined organic layers were washed with brine (30 mL), dried over Na₂SO₄, filtered and the filtrate was concentrated under reduced pressure to give a residue, which was purified by column chromatography on silica gel eluted with ethyl acetate (from 0% to 15%) in petroleum ether to give the intermediate 62-4 (550.0 mg, 51.5% yield) as yellow solid.

LC-MS (ESI): m/z 226.9 (M−H).

¹H NMR (400 MHz, CDCl₃) δ=6.58 (s, 1H), 5.19 (s, 2H), 5.11-5.06 (m, 2H), 2.33 (s, 3H).

To a solution of intermediate 62-4 (550.0 mg, 2.40 mmol) in acetonitrile (12 mL) was added NIS (1.08 g, 4.80 mmol). The reaction was stirred at rt for 1 h. To the reaction mixture was added saturated sodium sulfite aqueous solution (20 mL) and extracted with EtOAc (30 mL×2). The combined organic layers were washed with brine (30 mL), dried over Na₂SO₄, filtered and the filtrate was concentrated under reduced pressure to give a residue, which was purified by column chromatography on silica gel eluted with ethyl acetate (from 0% to 12%) in petroleum ether to give the intermediate 62-5 (720.0 mg, 84.5% yield) as yellow solid.

LC-MS(ESI): m/z 352.7 (M−H).

¹H NMR (400 MHz, CDCl₃) δ=5.26-5.19 (m, 2H), 5.08-5.02 (m, 2H), 2.65 (s, 3H).

To a solution of intermediate 62-5 (700.0 mg, 1.97 mmol) in toluene (20 mL) were added tributyl(1-ethoxyvinyl)stannane (783.0 mg, 2.17 mmol) and Pd(PPh₃)₄ (46.0 mg, 0.039 mmol). The reaction was stirred at 120° C. for 16 h under N₂. To the cooled reaction mixture was added HCl (15.1 mL, 90.6 mmol, 6 mol/L in H₂O) and the mixture was stirred at rt for another 2 h. To the reaction mixture was added water (30 mL) and extracted with EtOAc (30 mL×2). The combined organic layers were washed with brine (50 mL), dried over Na₂SO₄, filtered and the filtrate was concentrated under reduced pressure to give a residue, which was purified by column chromatography on silica gel eluted with ethyl acetate (from 0% to 12%) in petroleum ether to give the intermediate 62-6 (300.0 mg, 56.2% yield) as yellow solid.

LC-MS(ESI+): m/z 271.0 (M+H)⁺.

¹H NMR (400 MHz, CDCl₃) δ=11.34 (s, 1H), 5.23 (s, 2H), 5.10-5.02 (m, 2H), 2.65 (s, 3H), 2.62 (s, 3H).

To a solution of intermediate 62-6 (2.60 g, 9.59 mmol) in DMF (30 mL) were added sodium formate (978.0 mg, 14.38 mmol) and Pd(PPh₃)₄ (665.0 mg, 0.58 mmol). The mixture was stirred at 95° C. for 16 h under H₂ atmosphere. To the cooled reaction mixture was added water (30 mL) and extracted with EtOAc (50 mL×2). The combined organic layers were washed with brine (50 mL), dried over Na₂SO₄, filtered and the filtrate was concentrated under reduced pressure to give a residue, which was purified by column chromatography on silica gel eluted with ethyl acetate (from 0% to 12%) in petroleum ether to give the intermediate 62-7 (1.00 g, 54.2% yield) as yellow solid.

LC-MS(ESI+): m/z 193.1 (M+H)⁺.

¹H NMR (400 MHz, CDCl₃) δ=12.66 (s, 1H), 6.63 (s, 1H), 5.11 (s, 2H), 5.08-5.00 (m, 2H), 2.67 (s, 3H), 2.62 (s, 3H).

### Example 53

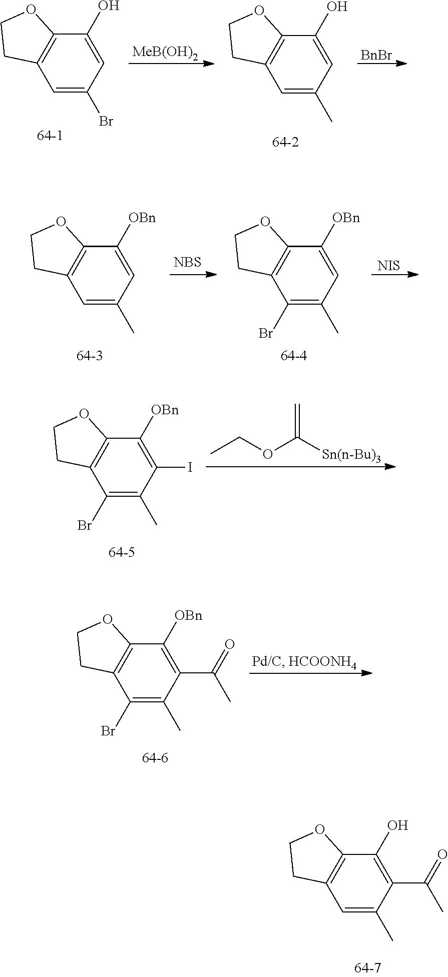

To a solution of compound 64-1 (4.54 g, 21.11 mmol)) in 1,4-Dioxane (50 mL) were added K₃PO₄ (8.96 g, 42.20 mmol), methylboronic acid (7.60 g, 127.00 mmol), Pd(OAc)₂ (143.0 mg, 0.64 mmol) and S-Phos (521.4 mg, 1.27 mmol). The mixture was stirred at 110° C. for 16 h under Ar. The mixture was concentrated to give the residue, which was purified by column chromatography on silica gel eluted with ethyl acetate (from 0% to 75%) in petroleum ether to give the intermediate 64-2 (2.50 g, 78.9% yield) as white solid.

LC-MS(ESI+): m/z 151.1 (M+H)⁺

¹H NMR: (400 MHz, CDCl₃) δ=6.59 (s, 1H), 6.55 (s, 1H), 4.58 (t, J=8.8 Hz, 2H), 3.19 (t, J=8.8 Hz, 2H), 2.24 (s, 3H).

To a solution of intermediate 64-2 (1.10 g, 7.32 mmol) in DMF (10 mL) were added benzyl bromide (1.50 g, 8.79 mmol) and K₂CO₃ (3.0 g, 21.97 mmol) at rt. The reaction was stirred at rt for 16 h. To the reaction mixture was added water (20 mL) and extracted with EtOAc (20 mL×2). The combined organic layers were washed with brine (20 mL), dried over Na₂SO₄, filtered and the filtrate was concentrated under reduced pressure to give a residue, which was purified by column chromatography on silica gel eluted with ethyl acetate (from 0% to 15%) in petroleum ether to give the intermediate 64-3

(1.70 g, 96.6% yield) as white solid.

¹H NMR: (400 MHz, CDCl₃) δ=7.45-7.29 (m, 5H), 6.65 (s, 1H), 6.59 (s, 1H), 5.12 (s, 2H), 4.60 (t, J=8.8 Hz, 2H), 3.18 (t, J=8.8 Hz, 2H), 2.24 (s, 3H).

To a solution of intermediate 64-3 (1.70 g, 7.07 mmol) in DCM (20 mL) was added NBS (1.66 g, 9.34 mmol) at 0° C. The reaction was stirred at rt for 16 h. The mixture was concentrated under reduced pressure to give a residue, which was purified by column chromatography on silica gel eluted with ethyl acetate (from 0% to 7%) in petroleum ether to give the intermediate 64-4 (2.20 g, 97.5% yield) as white solid.

¹H NMR: (400 MHz, CDCl₃) δ=7.50-7.30 (m, 5H), 6.67 (s, 1H), 5.11 (s, 2H), 4.65 (t, J=8.8 Hz, 2H), 3.22 (t, J=8.8 Hz, 2H), 2.27 (s, 3H).

To a solution of intermediate 64-4 (2.20 g, 6.89 mmol) in DCM (25 mL) was added NIS (1.55 g, 6.89 mmol) at 0° C. The mixture was stirred at rt for 10 min. To the mixture was added silver trifluoromethanesulfonate (0.91 g, 3.54 mmol) and the reaction was stirred at rt for 3 h. To the reaction mixture was added water (50 mL) and extracted with EtOAc (50 mL×2). The combined organic layers were washed with brine (50 mL), dried over Na₂SO₄, filtered and the filtrate was concentrated under reduced pressure to give a residue, which was purified by column chromatography on silica gel eluted with ethyl acetate (from 0% to 6%) in petroleum ether to give the intermediate 64-5 (2.40 g, 78.3% yield) as white solid.

¹H NMR: (400 MHz, CDCl₃) δ=7.56-7.32 (m, 5H), 5.13 (s, 2H), 4.65 (t, J=8.8 Hz, 2H), 3.22 (t, J=8.8 Hz, 2H), 2.62 (s, 3H).

To a solution of intermediate 64-5 (2.35 g, 5.28 mmol) in Toluene (25 mL) were added tributyl(1-ethoxyvinyl)stannane (2.29 g, 6.34 mmol) and Pd(PPh₃)₄ (122.0 mg, 0.11 mmol). The reaction was stirred at 120° C. for 16 h under Ar. To the cooled reaction mixture was added HCl (50 mL, 200 mmol) (4 M) and the mixture was stirred at rt for another 2 h. To the reaction mixture was added water (50 mL) and extracted with EtOAc (50 mL×2). The combined organic layers were washed with brine (50 mL), dried over Na₂SO₄, filtered and the filtrate was concentrated under reduced pressure to give a residue, which was purified by column chromatography on silica gel eluted with ethyl acetate (from 0% to 6%) in petroleum ether to give the intermediate 64-6 (390.0 mg, 20.4% yield) as colorless oil.

¹H NMR: (400 MHz, CDCl₃) δ=7.35-7.25 (m, 5H), 5.05 (s, 2H), 4.59 (t, J=8.8 Hz, 2H), 3.17 (t, J=8.8 Hz, 2H), 2.31 (s, 3H), 2.10 (s, 3H).

To a solution of intermediate 64-6 (160.0 mg, 0.44 mmol) in EtOH/Dioxane (1 mL/1 mL) were added TEA (0.06 mL, 0.44 mmol), 10% Pd/C (24 mg, 50% in water) and ammonium formate (17.0 mg, 0.27 mmol). The reaction was stirred at 60° C. for 16 h under H₂ balloon. The mixture was filtered and the filtrate was concentrated under reduced pressure to give a residue, which was purified by column chromatography on silica gel eluted with ethyl acetate (from 0% to 10%) in petroleum ether to give the intermediate 64-7 (85.0 mg, 100% yield) as yellow solid.

LC-MS(ESI+): m/z 193.1 (M+H)⁺.

¹H NMR: (400 MHz, CDCl₃) δ=11.77 (s, 1H), 6.62 (s, 1H), 4.64 (t, J=9.2 Hz, 2H), 3.21 (t, J=9.2 Hz, 2H), 2.64 (s, 3H), 2.52 (s, 3H).

### Example 54

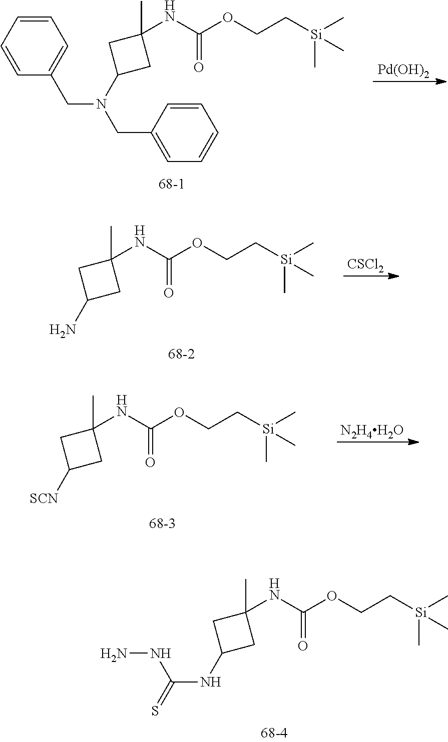

To a solution of compound 68-1 (0.70 g, 1.65 mmol) in MeOH (10 mL) was added Pd (OH) 2 (579 mg, 0.82 mmol). The reaction was heated to 30° C. and stirred at this temperature for 16 hr under H₂ balloon. The mixture was filtered and the filtrate was concentrated under reduced pressure to give the intermediate 68-2 (0.40 g, 99.2% yield) as a colorless oil.

LC-MS(ESI+): m/z 245.1 (M+H)⁺.

To a mixture of CaCO₃ (184.0 mg, 1.84 mmol) in DCM (6 mL) and Water (3 mL) were slowly added intermediate 68-2 (150 mg, 0.61 mmol) and thiophosgene (105.8 mg, 0.92 mmol) at 0° C. under N₂. After the addition, the reaction was stirred at rt for 1 h. The mixture was filtered and the filtrate was extracted with DCM (30 mL×3) and water (30 mL). The combined organic layers were dried over Na₂SO₄, filtered and the filtrate was concentrated under reduced pressure to give the intermediate 68-3 (150.0 mg, 85.8% yield) as yellow oil, which was used for next step without further workup. To a solution of intermediate 68-3 (150.0 mg, 0.52 mmol) in MeOH (5 mL) was added hydrazinium hydroxide solution (46 mg, 0.78 mmol, 85 wt %) under N₂. The reaction was stirred at rt for 1 h. The mixture was concentrated under reduced pressure to give the residue, which was lyophilized to give the intermediate 68-4 (140.0 mg, 84.5% yield) as white solid.

LC-MS(ESI+): m/z 319.1 (M+H)⁺.

The compounds below were prepared using a synthesis method similar to that described in Compound 1 or Compound 2 by substituting the appropriate starting materials, reagents and reaction conditions. The reaction temperatures varied from −78° C. to 0° C. for last step under the condition of BBr₃.

| Compound | |
|---|---|
| No. | Analytical data |
| 32 | LC-MS (ESI+): m/z: 361.1 (M + H)⁺. |
| | ¹H NMR (400 MHz, DMSO-d₆) δ = 10.50 (br. s, 1H), 7.92-7.75 (m, 1H), 7.51-7.16 |
| | (m, 1H), 7.08 (s, 1H), 7.01 (s, 1H), 3.89 (s, 2H), 3.45-3.36 (m, 2H), 2.62 (d, J = |
| | 4.8 Hz, 3H), 2.28 (s, 3H). |
| 33 | Prep-HPLC: (Column: C18 150 × 30 mm, Mobile Phase A: water (FA), Mobile Phase |
| and | B: acetonitrile, Flow rate: 30 mL/min, gradient condition from 15% B to 45%) |
| 34 | First peak, LC-MS (ESI+): m/z 388.1 (M + H)⁺. |
| | ¹H NMR (400 MHz, DMSO-d₆) δ = 10.52 (br. s, 1H), 7.08 (s, 1H), 7.01 (s, 1H), |
| | 3.98-3.85 (m, 2H), 3.70-3.60 (m, 2H), 3.45-3.40 (m, 1H), 3.29-3.18 (m, 1H), |
| | 2.31 (s, 3H), 1.94-1.85 (m, 4H), 1.02 (d, J = 6.4 Hz, 3H). |
| | Second peak, LC-MS (ESI+): m/z 388.1 (M + H)⁺. |
| | ¹H NMR (400 MHz, DMSO-d₆) δ = 10.90 (br. s, 1H), 7.16 (s, 1H), 7.07 (s, 1H), |
| | 4.18-4.03 (m, 2H), 3.98-3.81 (m, 2H), 3.79-3.66 (m, 2H), 2.34 (s, 3H), |
| | 2.21-2.07 (m, 2H), 2.03-1.88 (m, 2H), 1.09 (d, J = 6.4 Hz, 3H). |
| 35 | LC-MS (ESI+): m/z 413.1 (M + H)⁺. |
| | ¹H NMR (400 MHz, DMSO-d₆) δ = 8.19 (s, 1H, from HCOOH), 7.07 (s, 1H), 7.01 |
| | (s, 1H), 4.13-4.04 (m, 1H), 3.67-3.62 (m, 1H), 3.57-3.51 (m, 1H), 3.47-3.26 |
| | (m, 2H), 3.13-2.88 (m, 1H), 2.64-2.54 (m, 1H), 2.42-2.32 (m, 1H), 2.29 (s, 3H), |
| | 2.23 (s, 3H), 2.16-2.07 (m, 1H), 2.07-1.99 (m, 1H), 1.99-1.87 (m, 2H), |
| | 1.86-1.77 (m, 1H), 1.71-1.62 (m, 1H). |
| 37 | LC-MS (ESI+): m/z: 344.2 (M + H)⁺. |
| | ¹H NMR (400 MHz, DMSO-d₆) δ = 7.20 (s, 1 H), 7.06 (s, 1 H), 4.03-3.87 (m, 1 |
| | H), 3.48-3.34 (m, 2 H), 2.97-2.84 (m, 1 H), 2.65-2.55 (m, 1 H), 2.25 (s, 3 H), |
| | 2.16 (s, 3 H), 1.90-1.76 (m, 3 H), 1.72-1.61 (m, 1 H), 1.55-1.45 (m, 1 H), |
| | 1.29-1.17 (m, 1 H). |
| 41 | LC-MS(ESI+): m/z 378.0 (M + H)⁺. |
| | ¹H NMR (400 MHz, DMSO-d₆) δ = 7.21-7.12 (m, 1 H), 7.08 (s, 1 H), 5.00 (s, 1 |
| | H), 4.03-3.98 (m, 1 H), 3.78 (s, 2 H), 2.35-2.30 (m, 2 H), 2.05-2.00 (m, 2 H), |
| | 1.25 (s, 3 H). |
| 42 | LC-MS(ESI+): m/z 424.1 (M + H)⁺. |
| | ¹H NMR (400 MHz, DMSO-d₆) δ = 7.61 (s, 1 H), 7.53-7.16 (m, 3 H), 4.97 (s, 1 |
| | H), 4.04-3.90 (m, 1 H), 3.39 (s, 2 H), 2.35 (s, 3 H), 2.34-2.29 (m, 2 H), |
| | 2.04-1.99 (m, 2 H), 1.25 (s, 3 H). |
| 43 | LC-MS(ESI+): m/z 361.0 (M + H)⁺. |
| | ¹H NMR (400 MHz, DMSO-d₆) δ = 10.46 (br. s, 1 H), 7.56-6.68 (m, 5H), |
| | 3.60-3.48 (m, 2 H), 3.37 (s, 2 H), 2.41 (t, J = 7.2 Hz, 2 H), 2.29 (s, 3 H). |
| 45 | LC-MS(ESI+): m/z 394.0 (M + H)⁺. |
| | ¹H NMR (400 MHz, DMSO-d₆) δ = 7.34 (s, 1 H), 7.15 (s, 1 H), 4.97 (s, 1 H), 4.02-3.90 |
| | (m, 1 H), 3.43 (s, 2 H), 2.34-2.29 (m, 2 H), 2.04-1.99 (m, 2 H), 1.25 (s, 3 H). |
| 46 | LC-MS(ESI+): m/z 389.1 (M + H)⁺. |
| | ¹H NMR (400 MHz, CDCl₃) δ = 7.54 (d, J = 5.2 Hz, 1 H), 7.25-7.20 (m, 2 H), |
| | 4.81-4.70 (m, 1 H), 3.70-3.60 (m, 1 H), 3.56 (s, 2 H), 3.48-3.39 (m, 1 H), |
| | 2.99-2.91 (m, 2 H), 2.91-2.78 (m, 1 H), 2.66-2.62 (m, 1 H), 2.51 (s, 3 H), 2.41-2.35 |
| | (m, 1 H), 2.21-2.17 (m, 1 H), 1.89-1.85 (m, 1 H), 1.78-1.71 (m, 1 H), |
| | 1.45-1.30 (m, 3 H). |
| 47 | LC-MS(ESI+): m/z 405.1 (M + H)⁺. |
| | ¹H NMR (400 MHz, CDCl₃) δ = 8.12 (s, 1H, from HCOOH), 7.56 (d, J = 5.6 Hz, 1 |
| | H), 7.30-7.26 (m, 2 H), 4.80-4.72 (m, 1 H), 4.05-3.98 (m, 2 H), 3.70-3.62 (m, |
| | 1 H), 3.60 (s, 2 H), 3.58-3.50 (m, 1 H), 3.21-3.07 (m, 2 H), 2.90-2.82 (m, 1 H), |
| | 2.50 (s, 3 H), 2.48-2.42 (m, 1 H), 2.41-2.35 (m, 1 H), 2.21-2.07 (m, 2 H), |
| | 1.98-1.90 (m, 1 H), 1.87-1.80 (m, 1 H). |
| 48 | LC-MS(ESI+): m/z 392.0 (M + H)⁺. |
| | ¹H NMR (400 MHz, DMSO-d₆) δ = 7.12-7.05 (m, 1 H), 4.96 (br. s, 1 H), 4.07-3.90 |
| | (m, 1 H), 3.41 (s, 2 H), 2.33-2.29 (m, 2 H), 2.25 (s, 3 H), 2.04-1.99 (m, 2 H), |
| | 1.25 (s, 3 H). |
| 49 | LC-MS(ESI+): m/z 345.3 (M + H)⁺. |
| | ¹H NMR (400 MHz, DMSO-d₆) δ = 8.22 (s, 1H, from HCOOH), 7.22 (s, 1 H), 6.67 |
| | (s, 1 H), 4.53 (t, J = 8.4 Hz, 2 H), 3.95-3.92 (m, 1 H), 3.61-3.50 (m, 2 H), 3.17-3.10 |
| | (m, 2 H), 2.89-2.81 (m, 1 H), 2.68-2.64 (m, 1 H), 2.29 (s, 3 H), 2.18 (s, 3 H), |
| | 1.88-1.81 (m, 3 H), 1.69-1.65 (m, 1 H), 1.52-1.48 (m, 1 H), 1.29-1.19 (m, 1H). |
| 50 | LC-MS(ESI+): m/z 346.1 (M + H)⁺. |
| | ¹H NMR (400 MHz, DMSO-d₆) δ = 9.59 (br. s, 1 H), 6.65 (s, 1 H), 5.06 (br. s, 1 H), |
| | 4.02-3.92 (m, 1 H), 3.68-3.59 (m, 2 H), 2.82-2.75 (m, 4 H), 2.36-2.33 (m, 2 |
| | H), 2.20 (s, 3 H), 2.11-1.95 (m, 4 H), 1.26 (s, 3 H). |
| 53 | LC-MS(ESI+): m/z 388.0 (M + H)⁺. |
| | ¹H NMR (400 MHz, DMSO-d₆) δ = 7.10 (s, 1 H), 4.03-3.90 (m, 1 H), 3.53 (s, 2 |
| | H), 2.35-2.27 (m, 5 H), 2.25 (s, 3 H), 2.08-1.95 (m, 2 H), 1.26 (s, 3 H). |
| 56 | LC-MS(ESI+): m/z 357.1 (M + H)⁺. |
| | ¹H NMR (400 MHz, DMSO-d₆) δ = 10.01 (br. s, 1 H), 6.74 (s, 1 H), 6.71 (s, 1 H), |
| | 3.95-3.94 (m, 1 H), 3.39-3.32 (m, 2 H), 3.00-2.92 (m, 1 H), 2.66-2.60 (m, 1 |
| | H), 2.21 (s, 3 H), 2.18 (s, 3 H), 2.02 (s, 3 H), 1.95-1.81 (m, 3 H), 1.71-1.67 (m, 1 |
| | H), 1.57-1.46 (m, 1 H), 1.30-1.19 (m, 1 H). |
| 57 | LC-MS(ESI+): m/z 379.0 (M + H)⁺. |
| | ¹H NMR (400 MHz, DMSO-d₆) δ = 8.18 (s, 1H, from HCOOH), 4.63 (t, J = 8.4 |
| | Hz, 2 H), 4.02-3.92 (m, 1 H), 3.47-3.38 (m, 2 H), 3.15 (t, J = 8.8 Hz, 2 H), 3.08-2.97 |
| | (m, 1 H), 2.76-2.71 (m, 1 H), 2.27 (s, 3 H), 2.12 (s, 3 H), 2.06-1.90 (m, 2 H), |
| | 1.88-1.82 (m, 1 H), 1.73-1.65 (m, 1 H), 1.55-1.48 (m, 1 H), 1.29-1.22 (m, 1 |
| | H). |
| 58 | LC-MS(ESI+): m/z 402.1 (M + H)⁺ |
| | ¹H NMR (400 MHz, CD₃CN) δ = 6.74 (s, 1 H), 4.67 (t, J = 8.8 Hz, 2 H), 4.05-3.98 |
| | (m, 1 H), 3.48 (s, 2 H), 3.22 (t, J = 8.4 Hz, 2 H), 2.47-2.42 (m, 2 H), 2.09-2.01 |
| | (m, 2 H), 1.30 (s, 3 H). |
| 59 | LC-MS(ESI+): m/z 332.1 (M + H)⁺ |
| | ¹H NMR (400 MHz, CD₃CN) δ = 6.56 (s, 1 H), 4.05-3.97 (m, 1 H), 3.49 (s, 2 H), |
| | 3.16-3.01 (m, 4 H), 2.50-2.43 (m, 2 H), 2.30 (s, 3 H), 2.14-2.05 (m, 2H), 1.31 |
| | (s, 3 H). |
| 60 | LC-MS (ESI+): m/z 368.1 (M + H)⁺ |
| | ¹H NMR (400 MHz, CD₃CN) δ = 6.51 (s, 1 H), 4.64 (t, J = 8.4 Hz, 2 H), 4.11-4.00 |
| | (m, 1 H), 3.75 (s, 2 H), 3.13 (t, J = 8.8 Hz, 2 H), 2.52-2.41 (m, 2 H), |
| | 2.07-2.00 (m, 2 H), 1.31 (s, 3 H). |
| 61 | LC-MS (ESI+): m/z 366.1 (M + H)⁺ |
| | ¹H NMR (400 MHz, CD₃CN) δ = 4.69 (t, J = 8.8 Hz, 2 H), 4.08-3.96 (m, 1 H), |
| | 3.53 (s, 2 H), 3.17 (t, J = 8.8 Hz, 2 H), 2.52-2.42 (m, 2 H), 2.26-2.22 (m, 3 H), |
| | 2.07-2.00 (m, 2 H), 1.31 (s, 3 H). |
| 67 | LC-MS (ESI+): m/z 332.1 (M + H)⁺ |
| | ¹H NMR (400 MHz, CD₃CN) δ = 6.55 (s, 1H), 4.52-4.44 (m, 1H), 3.46 (s, 2H), |
| | 3.10-3.00 (m, 4H), 2.48-2.40 (m, 2H), 2.31 (s, 3H), 2.06-2.01 (m, 2H), 1.93 (s, |
| | 3H). |
| 68 | LC-MS (ESI+): m/z 331.2 (M + H)⁺ |
| | ¹H NMR (400 MHz, CD₃CN) δ = 8.18 (s, 1H from HCOOH), 6.55 (s, 1H), 4.24-4.15 |
| | (m, 1H), 3.46 (s, 2H), 3.12-3.02 (m, 4H), 2.50-2.41 (m, 2H), 2.30 (s, 3H), |
| | 2.12-2.01 (m, 2H), 1.29 (s, 3H). |
| 69 | LC-MS (ESI+): m/z 331.1 (M + H) ⁺ |

The compounds below were prepared using a synthesis method similar to that described in Compound 25 by substituting the appropriate starting materials, reagents and reaction conditions.

| Compound | |
|---|---|
| No. | Analytical data |
| 38 | LC-MS (ESI+): m/z 324.1 (M + H)⁺. |
| | ¹H NMR (400 MHz, DMSO-d₆) δ = 10.42 (br. s, 1H), 6.60-6.47 (m, 2H), |
| | 4.96 (s, 1H), 3.99-3.88 (m, 1H), 3.36-3.35 (m, 2H), 2.36-2.26 (m, 2H), |
| | 2.21 (s, 3H), 2.04-1.96 (m, 2H), 1.24 (s, 3H). |
| 39 | LC-MS (ESI+): m/z: 370.1 (M + H)⁺. |
| | ¹H NMR (400 MHz, DMSO-d₆) δ = 7.21 (s, 1 H), 7.06 (s, 1 H), 4.14-3.98 |
| | (m, 1 H), 3.68-3.62 (m, 1 H), 3.58-3.50 (m, 2 H), 3.32-3.23 (m, 1 H), |
| | 2.97-2.87 (m, 1 H), 2.40-2.31 (m, 1 H), 2.26 (s, 3 H), 2.17 (s, 3 H), |
| | 2.07-1.78 (m, 6 H), 1.69-1.61 (m, 1 H). |

### Example 55

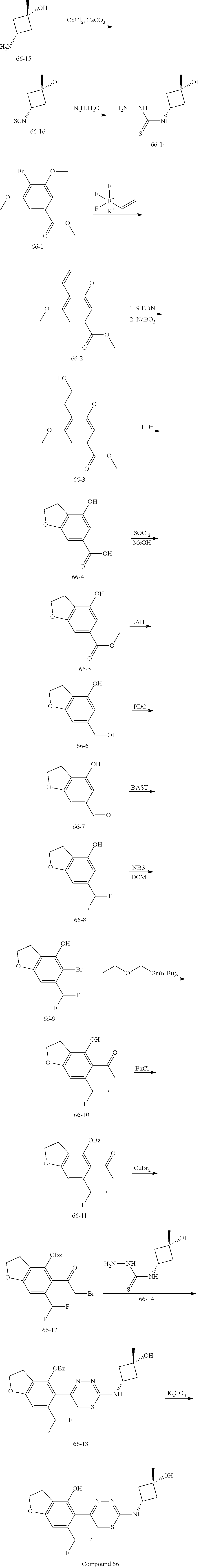

To a mixture of CaCO₃ (37.10 g, 370.68 mmol) in DCM (150 mL) and H₂O (75 mL) were added compound 66-15 (17.00 g, 123.54 mmol) and thiophosgene (28.4 g, 247.02 mmol) at 0° C. under N₂. The resulting mixture was stirred at rt for 16 h. The reaction mixture was filtered and the filtrate was extracted with DCM (200 mL×3). The combined organic layers were washed with brine (100 mL), dried over Na₂SO₄, filtered and concentrated under reduced pressure to give the intermediate 66-16 (15.00 g, 104.75 mmol, 85% yield) as yellow oil, which was used in the next step without further purification.

¹H NMR: (400 MHz, DMSO-d₆) δ=3.99-3.80 (m, 1H), 2.46-2.32 (m, 2H), 2.24-2.09 (m, 2H), 1.00 (s, 3H).

To a solution of intermediate 66-16 (15.00 g, 104.75 mmol) in MeOH (100 mL) was added hydrazine hydrate (7.40 g, 125.65 mmol, 85 wt %) under N₂. The mixture was stirred at rt for 1 h. The mixture was concentrated under reduced pressure to give the intermediate 66-14 (17.70 g, 96.4% yield) as yellow solid.

¹H NMR: (400 MHz, DMSO-d₆) δ=8.64 (s, 1H), 7.73-7.62 (m, 1H), 4.94 (s, 1H), 4.48 (s, 2H), 4.29-4.18 (m, 1H), 2.37-2.24 (m, 2H), 2.01-1.94 (m, 2H), 1.21 (s, 3H).

To a solution of compound 66-1 (10.00 g, 36.35 mmol) in Dioxane/Water (150 mL/30 mL) were added potassium trifluoro (vinyl) borate (6.30 g, 47.03 mmol), Pd(dppf)Cl₂ (2.70 g, 3.69 mmol) and K₂CO₃ (12.6 g, 91.17 mmol). The mixture was stirred at 100° C. for 16 h. To the cooled reaction mixture was added water (100 mL) and extracted with EtOAc (100 mL×2). The combined organic layers were washed with brine (100 mL), dried over Na₂SO₄, filtered and the filtrate was concentrated under reduced pressure to give a residue, which was purified by column chromatography on silica gel eluted with ethyl acetate (from 0% to 12%) in petroleum ether to give the intermediate 66-2 (7.70 g, 95.3% yield) as white solid.

¹H NMR (400 MHz, CDCl₃) δ=7.25 (s, 2H), 7.01-6.93 (m, 1H), 6.18 (dd, J=18.0, 2.8 Hz, 1H), 5.55 (dd, J=12.4, 2.8 Hz, 1H), 3.92 (s, 3H), 3.90 (s, 6H).

To a solution of intermediate 66-2 (8.10 g, 36.45 mmol) in THF (100 mL) was added 9-BBN (146 mL, 0.5M in THF, 73.00 mmol). The mixture was stirred at rt for 16 h, the reaction was diluted with THF/Water (50 mL/50 mL). To the mixture was added sodium perborate tetrahydrate (33.60 g, 218.38 mmol) and the mixture was vigorously stirred for 1 h. The suspension was diluted with saturated NaHCO₃ aqueous solution (50 mL) and filtered. The aqueous phase was extracted with EtOAc (100 mL×3). The combined organic phases were washed with brine (100 mL), dried over Na₂SO₄. The solvent was evaporated and the residue was purified by column chromatography on silica gel eluted with ethyl acetate (from 0% to 20%) in petroleum ether to give the intermediate 66-3 (8.60 g, 98.2% yield) as white solid.

¹H NMR (400 MHz, CDCl₃) δ=7.25 (s, 2H), 3.92 (s, 3H), 3.88 (s, 6H), 3.78-3.72 (m, 2H), 3.01 (t, J=6.8 Hz, 2H).

A solution of intermediate 66-3 (6.60 g, 27.47 mmol) in HOAc (35 mL) and HBr (48 wt % in water, 35 mL) was stirred at 120° C. for 16 h. To the cooled reaction mixture was added water (50 mL) and the mixture was extracted with EtOAc (50 mL×3). The combined organic layers were washed with brine (30 mL), dried over Na₂SO₄, filtered and the filtrate was concentrated under reduced pressure to give a residue, which was purified by column chromatography on silica gel eluted with MeOH (from 0% to 5%) in DCM to give the intermediate 66-4 (3.20 g, 64.7% yield) as yellow solid.

LC-MS(ESI+): m/z 181.1 (M+H)⁺

To a solution of intermediate 66-4 (1.00 g, 5.55 mmol) in MeOH (10 mL) was added sulfurous dichloride (2 mL) at 0° C. The reaction was stirred at 65° C. for 6 h. The solvent was evaporated to give the residue. The residue was dissolved in saturated NaHCO₃ aqueous solution (30 mL) and extracted with EtOAc (20 mL×2). The combined organic layers were washed with brine (20 mL), dried over Na₂SO₄, filtered and the filtrate was concentrated under reduced pressure to give a residue, which was purified by column chromatography on silica gel eluted with ethyl acetate (from 0% to 50%) in petroleum ether to give the intermediate 66-5 (0.37 g, 34.3% yield) as yellow solid.

¹H NMR (400 MHz, DMSO-d₆) δ=9.92 (s, 1H), 6.98 (s, 1H), 6.76 (s, 1H), 4.56 (t, J=8.8 Hz, 2H), 3.79 (s, 3H), 3.09 (t, J=8.8 Hz, 2H).

To a solution of intermediate 66-5 (680.0 mg, 3.50 mmol) in THF (10 mL) was added LiAlH₄ (292.0 mg, 7.69 mmol) at 0° C. under N₂. The reaction was stirred at rt for 1 h. The mixture was diluted with DCM (10 mL), added water (0.5 mL), 15% NaOH (0.5 mL) and water (1 mL) in sequence at 0° C. Na₂SO₄ was added to the mixture and the mixture was stirred at rt for 10 min. The suspension was filtered and the filtrate was concentrated under reduced pressure to give a residue, which was purified by column chromatography on silica gel eluted with ethyl acetate (from 0% to 50%) in petroleum ether to give the intermediate 66-6 (494.0 mg, 84.9% yield) as white solid.

¹H NMR (400 MHz, DMSO-d₆) δ=9.33 (s, 1H), 6.28 (s, 1H), 6.18 (s, 1H), 5.03 (t, J=5.6 Hz, 1H), 4.47 (t, J=8.8 Hz, 2H), 4.32 (d, J=5.6 Hz, 2H), 2.99 (t, J=8.8 Hz, 2H).

To a solution of intermediate 66-6 (0.60 g, 3.61 mmol) in DCM (10 mL) was added PDC (2.70 g, 7.18 mmol) in portions while maintaining the inner temperature at 0° C. After the addition, the reaction was stirred at rt for 1 h. The mixture was concentrated under reduced pressure to give a residue, which was purified by column chromatography on silica gel eluted with ethyl acetate (from 0% to 30%) in petroleum ether to give the intermediate 66-7 (0.21 g, 35.4% yield) as yellow solid.

¹H NMR (400 MHz, CDCl₃) δ=9.83 (s, 1H), 6.90-6.87 (m, 2H), 5.12 (s, 1H), 4.68 (t, J=8.8 Hz, 2H), 3.23 (t, J=8.8 Hz, 2H).

To a solution of intermediate 66-7 (0.26 g, 1.58 mmol) in DCM (5 mL) was added BAST (0.80 mL, 4.34 mmol) under N₂ at 0° C. The reaction was stirred at rt for 16 h. To the reaction mixture was added water (20 mL), saturated NaHCO₃ aqueous solution (5 mL) and the mixture was extracted with DCM (20 mL×2). The combined organic layers were washed with brine (10 mL), dried over Na₂SO₄, filtered and the filtrate was concentrated under reduced pressure to give a residue, which was purified by column chromatography on silica gel eluted with ethyl acetate (from 0% to 22%) in petroleum ether to give the intermediate 66-8 (0.23 g, 78.2% yield) as yellow solid.

¹H NMR (400 MHz, CDCl₃) δ=6.64-6.36 (m, 3H), 4.94 (br.s, 1H), 4.65 (t, J=8.4 Hz, 2H), 3.18 (t, J=8.8 Hz, 2H). ¹⁹F NMR (376 MHz, CDCl₃) δ=−110.16.

To a solution of intermediate 66-8 (230 mg, 1.23 mmol) in DCM (5 mL) was added NBS (0.22 g, 1.24 mmol) at −40° C. The reaction was stirred at rt for 2 h. To the reaction mixture was added water (20 mL) and the mixture was adjusted to pH=4 with HCl (2N) and then extracted with DCM (20 mL×2). The combined organic layers were washed with brine (20 mL), dried over Na₂SO₄, filtered and the filtrate was concentrated under reduced pressure to give a residue, which was purified by column chromatography on silica gel eluted with DCM (from 0% to 50%) in petroleum ether to give the intermediate 66-9 (0.28 g, 85.2% yield) as white solid.

LC-MS(ESI): m/z 263.0 (M−H)⁻.

To a solution of intermediate 66-9 (0.28 g, 1.06 mmol) in Toluene (5 mL) were added tributyl(1-ethoxyvinyl)stannane (459.0 mg, 1.27 mmol) and Pd(PPh₃)₄ (23.0 mg, 0.020 mmol) under Ar. After the addition, the reaction was stirred at 120° C. for 16 h. To the cooled reaction was added 4M HCl (5 mL) and the mixture was stirred at rt for another 2 h. To the reaction mixture was added water (20 mL) and extracted with EtOAc (20 mL×2). The combined organic layers were washed with brine (20 mL), dried over Na₂SO₄, filtered and the filtrate was concentrated under reduced pressure to give a residue, which was purified by column chromatography on silica gel eluted with ethyl acetate (from 0% to 15%) in petroleum ether to give the intermediate 66-10 (0.16 g, 66.1% yield) as yellow solid.

¹H NMR (400 MHz, CDCl₃) δ=12.87 (s, 1H), 7.06 (t, J=54.8 Hz, 1H), 6.76 (s, 1H), 4.74 (t, J=8.8 Hz, 2H), 3.22 (t, J=8.8 Hz, 2H), 2.66 (s, 3H).

To a solution of intermediate 66-10 (155.0 mg, 0.68 mmol) and TEA (69.0 mg, 0.68 mmol) in DCM (5 mL) were added DMAP (17.0 mg, 0.14 mmol) and benzoyl chloride (124.0 mg, 0.88 mmol). The reaction was stirred at rt for 1 h. The mixture was concentrated under reduced pressure to give a residue, which was purified by column chromatography on silica gel eluted with ethyl acetate (from 0% to 12%) in petroleum ether to give the intermediate 66-11 (220.0 mg, 97.4% yield) as yellow solid.

¹H NMR (400 MHz, CDCl₃) δ=8.25-8.12 (m, 2H), 7.71-7.65 (m, 1H), 7.57-7.50 (m, 2H), 7.05-6.75 (m, 2H), 4.70 (t, J=8.6 Hz, 2H), 3.16 (t, J=8.6 Hz, 2H), 2.46 (s, 3H).

To a solution of intermediate 66-11 (40.0 mg, 0.12 mmol) in EtOAc/DCE (1 mL/1 mL) was added copper(II) bromide (36.0 mg, 0.16 mmol). The reaction was stirred at 80° C. for 16 h. After cooling to room temperature, the reaction mixture was filtered and the filtrate was concentrated under reduced pressure to give a residue, which was purified by Pre-TLC (PE/EA=5/1) to give the crude intermediate 66-12 (49.0 mg) as white solid, which was used directly in the next step without further purification. To a solution of intermediate 66-12 (156.0 mg, 0.38 mmol) and intermediate 66-14 (67.0 mg, 0.38 mmol) in EtOH (5 mL) was added HBr (48 wt. % in water, 96 mg). After stirred at rt for 1 h, the reaction was stirred at 60° C. for 16 h. The solvent was removed under vacuum, and the residue was diluted with EA. To the mixture was added NH₄OH until pH=10 at 0° C. To the mixture was added H₂O (10 mL) and the mixture was extracted with EtOAc (20 mL×3). The combined organic layers were dried over Na₂SO₄, filtered and the filtrate was concentrated under reduced pressure to give a residue, which was purified by column chromatography on silica gel eluted with MeOH (from 0% to 4.9%) in DCM to give the intermediate 66-13 (105.0 mg, 56.7% yield) as yellow solid.

LC-MS(ESI+): m/z 488.1 (M+H)⁺.

To a solution of intermediate 66-13 (95.0 mg, 0.19 mmol) in MeOH (3 mL) was added K₂CO₃ (131.0 mg, 0.95 mmol) at rt. The mixture was stirred at 50° C. for 1 h. The reaction mixture was filtered and washed with MeOH. The filtrate was concentrated under reduced pressure to give a residue, which was purified by prep-HPLC (Waters 3767/QDA) Column: SunFire C18, 19*250 mm, 10 μm; Mobile Phase A: 0.1% FA/H₂O, B: ACN; flow rate: 20 mL/min; gradient: 15-25%; Retention Time: 6.7-7.8 of 17 min) to give the compound 66.

LC-MS(ESI+): m/z 384.0 (M+H)⁺.

¹H NMR (400 MHz, CD₃CN) δ=6.83 (t, J=55.2 Hz, 1H), 6.67 (s, 1H), 4.65 (t, J=8.8 Hz, 2H), 4.07-3.98 (m, 1H), 3.47 (s, 2H), 3.25-3.18 (m, 2H), 2.49-2.42 (m, 2H), 2.08-1.95 (m, 2H), 1.31 (s, 3H).

### Example 56

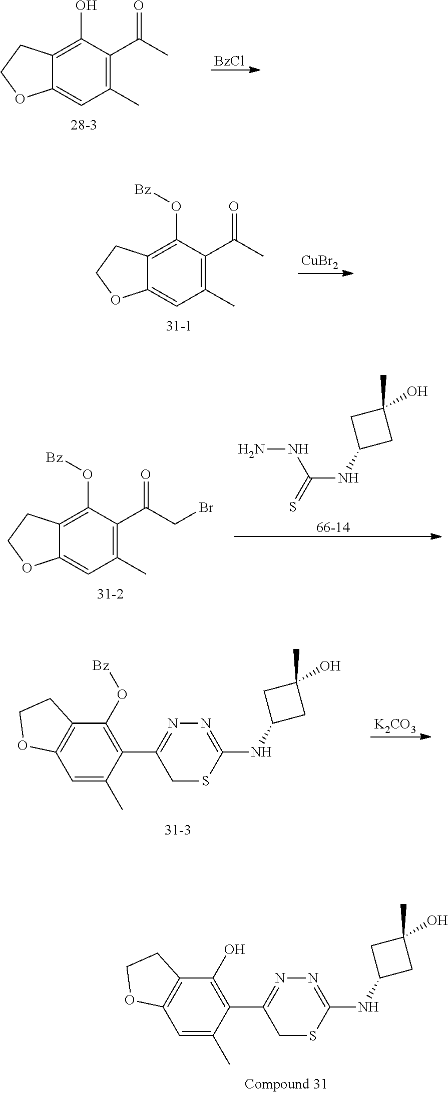

To a solution of intermediate 28-3 (1200 mg, 6.24 mmol) and TEA (632 mg, 6.24 mmol) in DCM (5 mL) were added 4-(dimethylamino)pyridine (153 mg, 1.25 mmol) and benzoyl chloride (1141 mg, 8.12 mmol). The reaction was stirred at rt for 2 h. The reaction mixture was concentrated under reduced pressure to give a residue, which was purified by column chromatography on silica gel eluted with 0˜4% ethyl acetate in petroleum ether to give the intermediate 31-1 (1561 mg, 84.4% yield) as white solid.

¹H NMR: (400 MHz, CDCl₃) δ=8.21-8.12 (m, 2H), 7.72-7.64 (m, 1H), 7.58-7.48 (m, 2H), 6.59 (s, 1H), 4.62 (t, J=8.8 Hz, 2H), 3.08 (t, J=8.8 Hz, 2H), 2.41 (s, 3H), 2.31 (s, 3H).

To a solution of intermediate 31-1 (500 mg, 1.69 mmol) in EtOAc (3 mL) and DCE (3 mL) was added CuBr₂ (490 mg, 2.19 mmol). The reaction was stirred at 80° C. for 16 h. The mixture was filtered and the filtrate was concentrated under reduced pressure to give a residue, which was purified by column chromatography on silica gel eluted with 0˜6% ethyl acetate in petroleum ether to give the intermediate 31-2 (500 mg, 79.0% yield) as yellow solid.

¹H NMR: (400 MHz, CDCl₃) δ=8.20-8.08 (m, 2H), 7.71-7.61 (m, 1H), 7.61-7.47 (m, 2H), 6.63 (s, 1H), 4.64 (t, J=8.4 Hz, 2H), 4.23 (s, 2H), 3.09 (t, J=8.8 Hz, 2H), 2.33 (s, 3H).

To a solution of intermediate 66-14 (300 mg, 1.71 mmol) in EtOH (10 mL) were added intermediate 31-2 (705 mg, 1.88 mmol) and conc. HCl (85 mg, 0.86 mmol, 37 wt %) at rt. After stirred at rt for 10 min, the mixture was stirred at 80° C. for 1 h. To the mixture was added NH₄OH until pH=10 at 0° C. Then the mixture was extracted with EtOAc (30 mL×3) and water (30 mL). The combined organic layers were washed with brine (30 mL), dried over Na₂SO₄, filtered and the filtrate was concentrated under reduced pressure to give a residue, which was purified by column chromatography on silica gel eluted with 0˜10% MeOH in DCM to give the intermediate 31-3 (230 mg, 29.8% yield) as red oil.

LC-MS(ESI+): m/z 452.2 (M+H)⁺.

To a solution of intermediate 31-3 (200 mg, 0.44 mmol) in MeOH (10 mL) was added K₂CO₃ (304 mg, 2.20 mmol) at rt. The mixture was stirred at 50° C. for 1 hr. Then the mixture was filtered and the filtrate was concentrated under reduced pressure to give a residue, which was purified by HPLC (column Waters Sun Fire Prep C18 OBD 10 μm 19*250 mm; mobile phase [0.1% FA in water-ACN]; B % 5%-95% ACN, 6.97 min) to give compound 31.

LC-MS (ESI+): m/z 348.0 (M+H)⁺.

¹H NMR (400 MHz, CD₃CN) δ=8.11 (s, 0.7H, from HCOOH), 6.26 (s, 1H), 4.57 (t, J=8.8 Hz, 2H), 4.07-3.98 (m, 1H), 3.55 (s, 2H), 3.10 (t, J=8.8 Hz, 2H), 2.49-2.43 (m, 2H), 2.32 (s, 3H), 2.11-2.00 (m, 2H), 1.31 (s, 3H).

The compounds below were prepared using a synthesis method similar to that described in Compound 66 by substituting the appropriate starting materials, reagents and reaction conditions.

| Compound | |
|---|---|
| No. | Analytical data |
| 40 | LC-MS (ESI+): m/z: 391.2 (M + H)⁺. |
| | ¹H NMR (400 MHz, DMSO-d₆) δ = 10.49 (br. s, 1H), 7.29-6.56 (m, 1H), |
| | 6.21 (s, 1H), 4.58-4.42 (m, 3H), 4.09-3.88 (m, 1H), 3.53-3.47 (m, 2H), |
| | 3.46-3.42 (m, 2H), 3.11-2.95 (m, 3H), 2.82-2.65 (m, 1H), 2.49-2.36 (m, |
| | 2H), 2.20 (s, 3H), 2.15-1.94 (m, 2H), 1.87-1.76 (m, 1H), 1.74-1.62 (m, |
| | 1H), 1.58-1.44 (m, 1H), 1.40-1.22 (m, 1H). |
| 44 | LC-MS(ESI+): m/z 331.2 (M + H)⁺. |
| | ¹H NMR (400 MHz, DMSO-d₆) δ = 10.46 (br. s, 1 H), 7.23 (s, 1 H), 7.09 (s, |
| | 1 H), 6.58-6.42 (m, 1H), 5.18-5.06 (m, 1 H), 5.03-4.92 (m, 1 H), 3.50-3.85 |
| | (m, 2 H), 2.45-2.40 (m, 2 H), 2.18 (s, 3 H), 2.15-2.08 (m, 2 H), 1.29 |
| | (s, 3 H). |
| 51 | LC-MS(ESI+): m/z 387.1 (M + H)⁺. |
| | ¹H NMR: (400 MHz, DMSO-d₆) δ = 10.42 (br. s, 1 H), 6.21 (s, 1 H), 4.51 (t, |
| | J = 8.4 Hz, 2 H), 4.06-3.96 (m, 1 H), 3.68-3.58 (m, 1 H), 3.56-3.50 (m, 2 |
| | H), 3.37 (s, 2 H), 3.06 (t, J = 8.4 Hz, 2 H), 2.94-2.85 (m, 1 H), 2.38-2.32 |
| | (m, 1 H), 2.20 (s, 3 H), 2.16 (s, 3 H), 2.04-1.95 (m, 2 H), 1.93-1.88 (m, 1 |
| | H), 1.87-1.81 (m, 2 H), 1.78-1.23 (m, 1 H). |
| 54 | LC-MS(ESI+): m/z 375.1 (M + H)⁺. |
| | ¹H NMR: (400 MHz, DMSO-d₆) δ = 10.47 (br. s, 1 H), 6.94-6.82 (m, 1 H), |
| | 6.21 (s, 1 H), 4.51 (t, J = 8.8 Hz, 2 H), 4.02-3.91 (m, 1 H), 3.49-3.38 (m, 2 |
| | H) 3.14-3.06 (m, 2 H), 3.03-2.98 (m, 1 H), 2.73-2.66 (m, 1 H), 2.36-2.32 |
| | (m, 2 H), 2.21 (s, 3 H), 2.02-1.94 (m, 1 H), 1.86-1.80 (m, 2 H), 1.68-1.62 |
| | (m, 1 H), 1.52-1.44 (m, 1 H), 1.42-1.29 (m, 1 H), 0.99 (t, J = 6.4 Hz, |
| | 3 H). |
| 55 | LC-MS(ESI+): m/z 375.1 (M + H)⁺. |
| | ¹H NMR (400 MHz, DMSO-d₆) δ = 10.40 (br. s, 1 H), 6.22 (s, 1 H), 4.52 (t, |
| | J = 8.8 Hz, 2 H), 4.32-4.21 (m, 1 H), 3.50-3.40 (m, 2 H), 3.06 (t, J = 8.8 |
| | Hz, 2 H), 3.03 (s, 3 H), 2.76-2.66 (m, 2 H), 2.21 (s, 3 H), 2.19 (s, 3 H), |
| | 2.05-1.99 (m, 2 H), 1.75-1.66 (m, 2 H), 1.60-1.52 (m, 2 H). |
| 62 | LC-MS(ESI+): m/z 348.0 (M + H)⁺. |
| | ¹H NMR (400 MHz, CD₃CN) δ = 6.71 (s, 1 H), 5.03-4.96 (m, 4 H), 4.08-4.04 |
| | (m, 1 H), 3.57 (s, 2 H), 2.52-2.45 (m, 2 H), 2.39 (s, 3 H), 2.15-2.08 |
| | (m, 2 H), 1.31 (s, 3 H). |
| 63 | LC-MS(ESI+): m/z 374.0 (M + H)⁺. |
| | ¹H NMR (400 MHz, CD₃CN) δ = 6.26 (s, 1 H), 4.58 (t, J = 8.4 Hz, 2 H), |
| | 3.53 (s, 2 H), 3.10 (t, J = 8.4 Hz, 2 H), 2.33 (s, 3 H), 1.80-1.58 (m, 10 H). |
| 64 | LC-MS(ESI+): m/z 348.1 (M + H)⁺. |
| | ¹H NMR: (400 MHz, CD₃CN) δ = 6.68 (s, 1 H), 4.56 (t, J = 8.4 Hz, 2 H), |
| | 4.02-3.95 (m, 1 H), 3.55 (s, 2 H), 3.19 (t, J = 8.8 Hz, 2 H), 2.53-2.45 (m, 2 |
| | H), 2.27 (s, 3 H), 2.11-2.02 (m, 2 H), 1.31 (s, 3 H). |
| 65 | LC-MS(ESI+): m/z 348.1 (M + H)⁺. |
| | ¹H NMR (400 MHz, CD₃CN) δ = 6.26 (s, 1 H), 4.58 (t, J = 8.4 Hz, 2 H), |
| | 4.55-4.48 (m, 1 H), 3.54 (s, 2 H), 3.10 (t, J = 8.8 Hz, 2 H), 2.44-2.38 (m, 2 |
| | H), 2.33 (s, 3 H), 2.06-2.01 (m, 2 H), 1.33 (s, 3 H). |
| 70 | LC-MS(ESI+): m/z 360.4 (M + H)⁺. |
| | ¹H NMR (400 MHz, CD₃CN) δ = 6.26 (s, 1H), 4.58 (t, J = 8.8 Hz, 2H), 3.54 |
| | (s, 2H), 3.10 (t, J = 8.8 Hz, 2H), 2.33 (s, 3H), 2.02-1.96 (m, 2H), |
| | 1.90-1.82 (m, 4H), 1.74-1.63 (m, 2H). |
| 71 | LC-MS(ESI+): m/z 351.3 (M + H)⁺. |
| | ¹H NMR (400 MHz, CD₃CN) δ = 6.25 (s, 1H), 4.57 (t, J = 8.8 Hz, 2H), 4.11-3.98 |
| | (m, 1H), 3.54 (s, 2H), 3.09 (t, J = 8.8 Hz, 2H), 2.52-2.40 (m, 2H), |
| | 2.05-1.98 (m, 2H), 1.31 (s, 3H). |

### Example 57

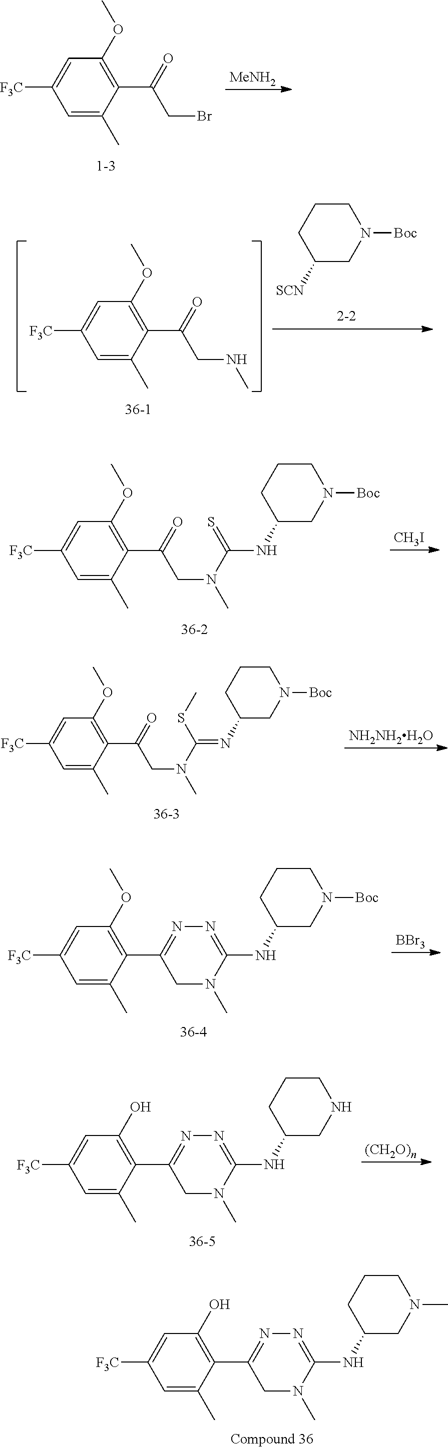

To MeNH₂ solution (6.9 mL, 13.80 mmol, 2 M in THF) was added a solution of intermediate 1-3 (1.0 g, 3.21 mmol) in THF (6.0 mL) at 0° C. and the mixture was stirred at 0° C. for 20 min to give intermediate 36-1. To the mixture were added TEA (0.45 mL, 3.22 mmol) and intermediate 2-2 (1.45 g, 5.99 mmol) at 0° C. and the mixture was stirred at 0° C. for 0.5 hour. The volatiles were removed in vacuo to afford a residue. The residue was purified by Prep-HPLC (Column: Xtimate C18 150*40 mm*10 μm, Mobile Phase A: water (0.05% NH₃H₂O+10 mM NH₄HCO₃), Mobile Phase B: acetonitrile, Flow rate: 55 mL/min, gradient condition from 60% B to 90%) to give the intermediate 36-2 (660.0 mg, 97.86% purity, 39.9% yield) as yellow solid.

LC-MS (ESI+): m/z 504.3 (M+H)⁺.

To a mixture of intermediate 36-2 (300.0 mg, 0.60 mmol) and K₂CO₃ (247.0 mg, 1.79 mmol) in acetone (4.0 mL) was added CH₃I (0.11 mL, 1.79 mmol) at 25° C. and the mixture was stirred at 25° C. for 1 hour. H₂O (30 mL) was added to the mixture and the mixture was extracted with ethyl acetate (20 mL×3). The combined organic extracts were dried over Na₂SO₄, filtered through a celite pad and the filtrate was concentrated under reduced pressure to give the intermediate 36-3 (310.0 mg, crude).

To a solution of intermediate 36-3 (310.0 mg, crude) in EtOH (3.0 mL) was added hydrazine hydrate (0.47 mL, 85% purity, 8.14 mmol) at 25° C. and the mixture was stirred at 50° C. for 6 hours. The mixture was concentrated under reduced pressure to afford a residue. The residue was purified by Prep-HPLC (Column: Welch Xtimate C18 150*30 mm*5 μm, Mobile Phase A: water (0.225% FA), Mobile Phase B: acetonitrile, Flow rate: 30 mL/min, gradient condition from 23% B to 53%) to give the intermediate 36-4 (130.0 mg, 86.26% purity, 38.9% yield over 2 steps) as white solid.

LC-MS (ESI+): m/z 484.0 (M+H)⁺

To a solution of intermediate 36-4 (90.0 mg, 0.19 mmol) in DCM (2.0 mL) was added BBr₃ (89.7 μL, 0.93 mmol) at 0° C. and the mixture was stirred at 0° C. for 1 hour and at 30° C. for 2 hours. Additional BBr₃ (89.7 μL, 0.91 mmol) was added at 30° C. and the mixture was stirred at 30° C. for another 1 hours. The mixture was quenched with MeOH (20.0 mL) at 0° C. and the volatiles were removed in vacuo to give the crude product. The crude product was purified by Prep-HPLC (Column: Welch Xtimate C18 150*30 mm*5 μm, Mobile Phase A: water (0.225% FA), Mobile Phase B: acetonitrile, Flow rate: 25 mL/min, gradient condition from 1% B to 31%), SFC (Column: DAICEL CHIRALPAK IG (250 mm*30 mm, 10 μm), Mobile Phase: CO₂-EtOH (0.1% NH₃H₂O), Flow rate: 80 mL/min, gradient condition from 40% to 40%) and by Prep-HPLC (Column: Welch Xtimate C18 150*30 mm*5 μm, Mobile Phase A: water (0.225% FA), Mobile Phase B: acetonitrile, Flow rate: 25 mL/min, gradient condition from 2% B to 32%) in sequence to give the intermediate 36-5 (3.0 mg, 4.3% yield) as brown oil.

LC-MS (ESI+): m/z 370.2 (M+H)⁺.

¹H NMR (400 MHz, DMSO-d₆) δ=7.06 (s, 1H), 7.02 (s, 1H), 4.00 (s, 2H), 3.83-3.80 (m, 1H), 3.17-3.10 (m, 1H), 2.96 (s, 3H), 2.95-2.91 (m, 1H), 2.65-2.56 (m, 2H), 2.30 (s, 3H), 1.94-1.85 (m, 1H), 1.79-1.70 (m, 1H), 1.58-1.46 (m, 2H).

To a solution of intermediate 36-5 (60.0 mg, 0.13 mmol) in MeOH (1.0 mL) was added TEA (36.9 μL, 0.27 mmol) at 20° C. Then HOAc (24.0 mg, 0.40 mmol) and (CH₂O)ₙ (12.0 mg, 0.40 mmol) were added to the mixture at 30° C. NaBH (OAc) 3 (112.4 mg, 0.53 mmol) was added at 30° C. and the mixture was stirred at 40° C. for 8 hours. Additional NaBH (OAc) 3 (112.4 mg, 0.53 mmol) was added at 40° C. and the mixture was stirred for another 3 hours. H₂O (20 mL) was added and the mixture was extracted with ethyl acetate (20 mL×3). The combined organic extracts were dried over Na₂SO₄, filtered through a celite pad and the filtrate was concentrated under reduced pressure to afford a crude. The crude was purified by SFC (Column: DAICEL CHIRALCEL OD (250 mm*30 mm, 10 μm), Mobile Phase: CO₂-EtOH (0.1% NH₃H₂O), Flow rate: 150 mL/min, gradient condition from 20% to 20%) and Prep-HPLC (Column: Welch Xtimate C18 150*30 mm*5 μm, Mobile Phase A: water (0.225% FA), Mobile Phase B: acetonitrile, Flow rate: 25 mL/min, gradient condition from 2% B to 32%) in sequence to give the compound 36.

LC-MS (ESI⁺): m/z 384.2 (M+H)⁺.

¹H NMR (400 MHz, DMSO-d₆) δ=7.11 (s, 1H), 7.01 (s, 1H), 4.13 (s, 2H), 3.87-3.78 (m, 1H), 3.07 (s, 3H), 2.89-2.79 (m, 1H), 2.70-2.61 (m, 1H), 2.30 (s, 3H), 2.21 (s, 3H), 2.09-1.99 (m, 1H), 1.96-1.81 (m, 2H), 1.73-1.50 (m, 2H), 1.41-1.28 (m, 1H).

### Example 58

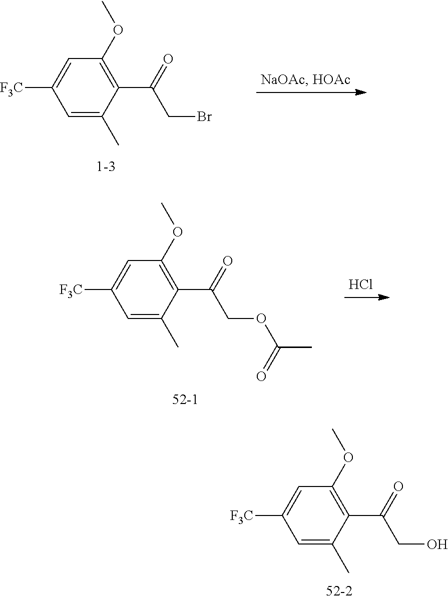 [SMILES: COc1cc(C(F)(F)F)cc(C)c1C(=O)CBr ; COc1cc(C(F)(F)F)cc(C)c1C(=O)COC(C)=O ; Cl ; COc1cc(C(F)(F)F)cc(C)c1C(=O)CO]

To a solution of intermediate 1-3 (2.00 g, 6.43 mmol) in EtOH (12 mL) was added a solution of NaOAc (580.0 mg, 7.07 mmol) in H₂O/HOAc (6 mL/0.6 mL). The reaction was stirred at 100° C. for 16 h. To the cooled reaction mixture was added water (20 mL) and the mixture was extracted with EtOAc (20 mL×2). The combined organic layers were washed with brine (20 mL), dried over Na₂SO₄, filtered and the filtrate was concentrated under reduced pressure to give a residue, which was purified by column chromatography on silica gel eluted with 0˜3.3% ethyl acetate in petroleum ether to give the intermediate 52-1 (1.10 g, 58.9% yield) as white solid.

¹H NMR: (400 MHz, CDCl₃) δ=7.11 (s, 1H), 6.98 (s, 1H), 4.99 (s, 2H), 3.88 (s, 3H), 2.33 (s, 3H), 2.16 (s, 3H).

To a solution of intermediate 52-1 (1.10 g, 3.79 mmol) in EtOH (10 mL) was added HCl (5 mL, 1 N). The reaction was stirred at 85° C. for 2 h. To the cooled reaction mixture was added water (20 mL) and the mixture was extracted with EtOAc (20 mL×2). The combined organic layers were washed with brine (20 mL), dried over Na₂SO₄, filtered and concentrated under reduced pressure to give a residue, which was purified by column chromatography on silica gel eluted with 0˜20% ethyl acetate in petroleum ether to give the intermediate 52-2 (700.0 mg, 2.82 mmol, 74.4% yield) as white solid.

¹H NMR: (400 MHz, CDCl₃) δ=7.12 (s, 1H), 6.99 (s, 1H), 4.63-4.52 (m, 2H), 3.87 (s, 3H), 3.23-3.12 (m, 1H), 2.29 (s, 3H).

### Example 59

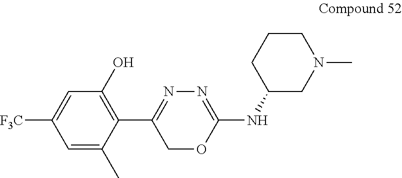 [SMILES: Cc1cc(C(F)(F)F)cc(O)c1C1=NN=C(N[C@@H]2CCCN(C)C2)OC1]

In a similar fashion according to the procedure for Compound 36, Compound 52 was synthesized by replacing intermediate 36-1 with intermediate 52-2. In the last step, NaBH₃CN was used instead, and the crude product was purified by Pre-HPLC (column: Waters Xbridge C18 10 μm OBD 19*250 mm; mobile phase: 0.1% NH₄HCO₃ in water, 9.088 min) to give compound 52.

LC-MS(ESI+): m/z 371.2 (M+H)⁺.

¹H NMR: (400 MHz, DMSO-d₆) δ=9.76 (br.s, 1H), 7.25-7.16 (m, 2H), 6.70-6.62 (m, 1H), 5.16 (s, 2H), 3.85-3.72 (m, 1H), 2.58 (s, 3H), 2.44-2.40 (m, 1H), 2.33-2.27 (m, 1H), 2.25-2.19 (m, 2H), 2.17 (s, 3H), 1.65-1.61 (m, 1H), 1.58-1.51 (m, 2H), 1.49-1.47 (m, 1H).

### Example 60

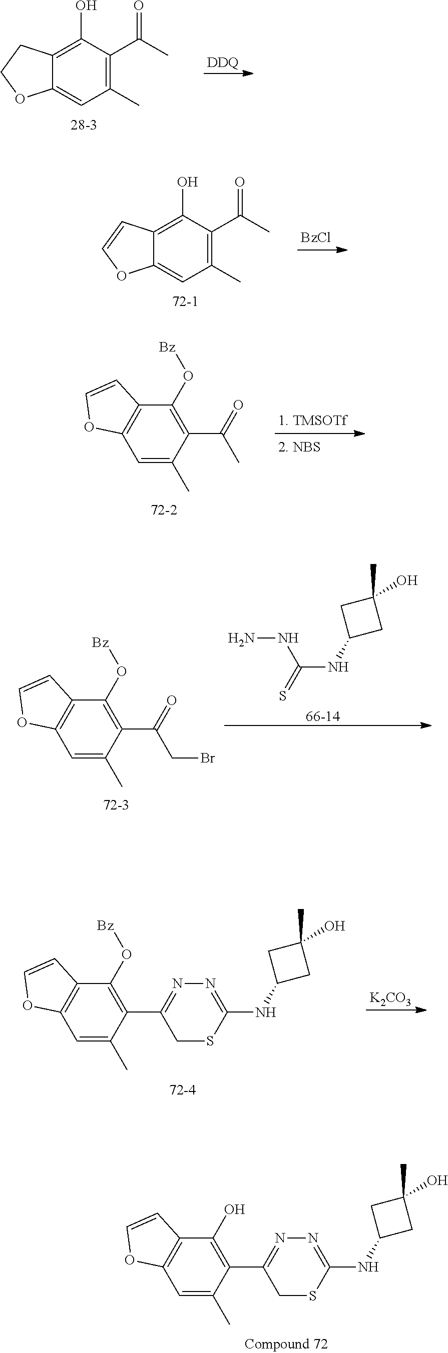

To a solution of intermediate 28-3 (164 mg, 0.85 mmol) in toluene (4 mL) was added DDQ (386 mg, 1.70 mmol) at rt. The reaction was stirred at 110° C. for 16 h. The reaction mixture was concentrated under reduced pressure to give a residue, which was purified by column chromatography on silica gel eluted with ethyl acetate (from 0% to 22%) in petroleum ether to give intermediate 72-1 (64 mg, 40% yield) as yellow solid.

¹H NMR (400 MHz, DMSO-d₆) δ=10.51 (s, 1H), 7.81 (d, J=2.0 Hz, 1H), 7.09 (s, 1H), 6.96 (s, 1H), 2.26 (s, 3H), 1.24 (s, 3H).

To a solution of intermediate 72-1 (85 mg, 0.45 mmol) and TEA (46 mg, 0.45 mmol) in DCM (2 mL) were added DMAP (11 mg, 0.09 mmol) and benzoyl chloride (83 mg, 0.59 mmol). The reaction was stirred at rt for 1 h. The reaction mixture was concentrated under reduced pressure to give a residue, which was purified by column chromatography on silica gel eluted with ethyl acetate (from 0% to 10%) in petroleum ether to give intermediate 72-2 (75 mg, 56% yield) as white solid.

LC-MS(ESI+): m/z 295.2 (M+H)⁺.

¹H NMR (400 MHz, CDCl₃) δ=8.26-8.21 (m, 2H), 7.73-7.65 (m, 1H), 7.58-7.50 (m, 3H), 7.31 (s, 1H), 6.60 (d, J=2.0 Hz, 1H), 2.50 (s, 3H), 2.44 (s, 3H).

To a solution of intermediate 72-2 (75 mg, 0.25 mmol) in DCM (3 mL) were added DIEA (39 mg, 0.30 mmol) and trimethylsilyl trifluoromethanesulfonate (67 mg, 0.30 mmol) at 0° C. After stirred at rt for 0.5 h, to the mixture was added NBS (53 mg, 0.30 mmol). The reaction mixture was stirred at rt for 2 h. The mixture was concentrated under reduced pressure to give a residue, which was purified by column chromatography on silica gel eluted with ethyl acetate (from 0% to 7%) in petroleum ether to give crude intermediate 72-3 (65 mg) as yellow oil, which was used directly in the next step.

To a solution of crude intermediate 72-3 (65 mg) and intermediate 66-14 (39 mg, 0.22 mmol) in EtOH (3 mL) was added HBr (38 mg, 0.23 mmol, 48 wt % in water). After stirred at rt for 1 h, the mixture was warmed to 60° C. and stirred for another 16 h. The solvent was removed under vacuum, and the mixture was diluted with EtOAc. To the mixture was added NH₄OH until pH=10 at 0° C. Then, to the mixture was added H₂O (10 mL) and extracted with EtOAc (10 mL×3). The combined organic layers were washed with brine (10 mL), dried over Na₂SO₄, filtered and the filtrate was concentrated under reduced pressure to give a residue, which was purified by pre-TLC (DCM/MeOH=10/1) to give intermediate 72-4 (18 mg, 16% yield over two steps) as yellow solid.

LC-MS(ESI+): m/z 450.1 (M+H)⁺.

To a solution of intermediate 72-4 (18 mg, 0.04 mmol) in MeOH (1 mL) was added K₂CO₃ (28 mg, 0.20 mmol). The reaction was stirred at 50° C. for 1 h. The reaction mixture was filtered and the filtrate was concentrated under reduced pressure to give a residue, which was purified by prep-HPLC (Waters 3767/QDA) Column: SunFire C18, 19*250 mm; 10 μm; Mobile Phase A: 0.1% FA/H₂O, B: ACN; flow rate: 20 mL/min; gradient: 12-22% Retention Time: 8.4-9.2 of 17 min) to give compound 72.

LC-MS(ESI+): m/z 345.9 (M+H)⁺.

¹H NMR (400 MHz, CD₃CN) δ=7.64-7.58 (m, 1H), 6.99 (s, 1H), 6.92-6.86 (m, 1H), 4.11-4.04 (m, 1H), 3.60 (s, 2H), 2.55-2.45 (m, 5H), 2.08-2.03 (m, 2H), 1.32 (s, 3H).

### Example 61

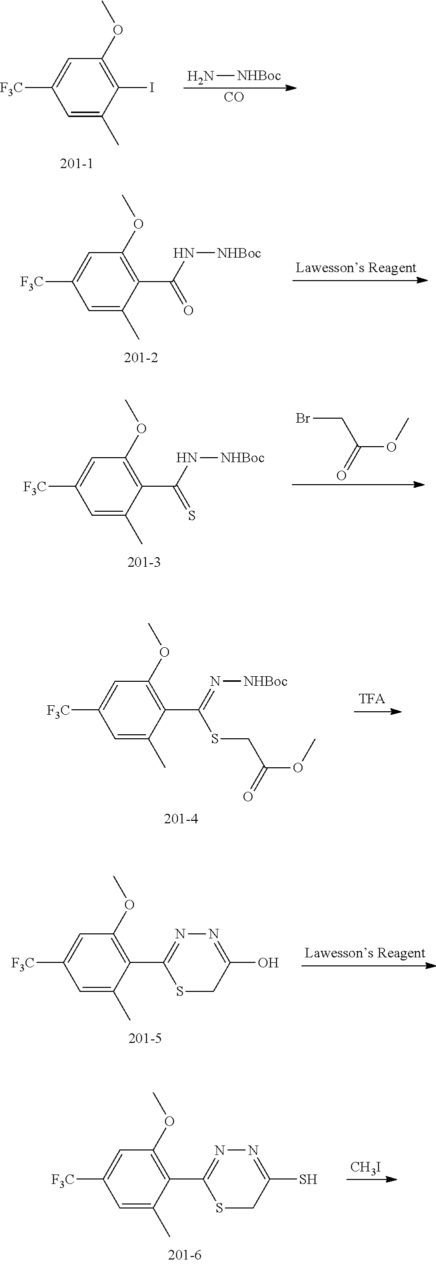

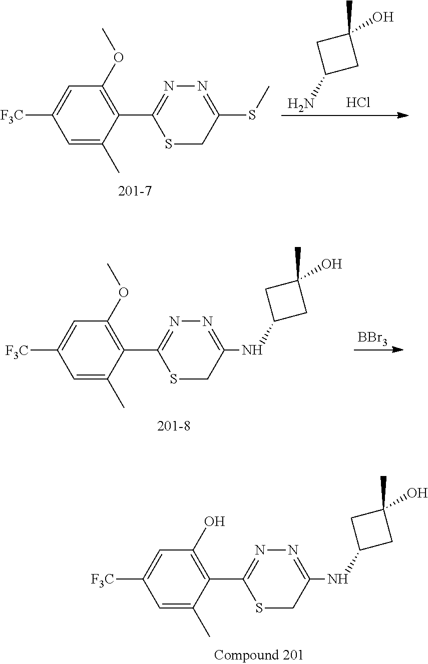

A solution of compound 201-1 (1.00 g, 3.16 mmol), Pd(OAc)₂ (142.0 mg, 0.63 mmol), tert-Butyl carbazate (627.0 mg, 4.74 mmol) and K₂CO₃ (874.0 mg, 6.32 mmol) in dry DMF (10 mL) was stirred at 90° C. for 16 h under CO atmosphere. To the cooled reaction mixture was added water (30 mL) and the mixture was extracted with EtOAc (30 mL×3). The combined organic layers were washed with brine (50 mL×3), dried over Na₂SO₄, filtered through a celite pad and the filtrate was concentrated under reduced pressure to give a residue, which was purified by column chromatography on silica gel eluted with ethyl acetate (from 0% to 8%) in petroleum ether to give the intermediate 201-2 (0.88 g, 79.9% yield) as white solid.

¹H NMR (400 MHz, CDCl₃) δ=7.62 (s, 1H), 7.11 (s, 1H), 6.97 (s, 1H), 6.70 (s, 1H), 3.87 (s, 3H), 2.46 (s, 3H), 1.46 (s, 9H).

A solution of intermediate 201-2 (0.83 g, 2.38 mmol) and Lawesson's Reagent (964.0 mg, 2.38 mmol) in dioxane (5 mL) was stirred at 85° C. for 16 h. The reaction mixture was concentrated under reduced pressure to give a residue, which was purified by column chromatography on silica gel eluted with ethyl acetate (from 0% to 20%) in petroleum ether to give the intermediate 201-3 (0.50 g, 57.7% yield) as yellow oil.

LC-MS(ESI+): m/z 309.0 (M+H−56)⁺.

To a solution of intermediate 201-3 (0.50 g, 1.37 mmol) in DMF (5 mL) were added methyl bromoacetate (0.14 mL, 1.51 mmol) and TEA (0.23 mL, 1.64 mmol). The mixture was stirred at 65° C. for 3 h. To the cooled reaction mixture was added water (20 mL) and the mixture was extracted with EtOAc (20 mL×3). The combined organic layers were washed with brine (30 mL×3), dried over Na₂SO₄, filtered through a celite pad and the filtrate was concentrated under reduced pressure to give a residue, which was purified by column chromatography on silica gel eluted with ethyl acetate (from 0% to 25%) in petroleum ether to give the intermediate 201-4 (200.8 mg, 33.6% yield) as yellow oil.

LC-MS(ESI+): m/z 381.0 (M+H−56)⁺.

To a solution of intermediate 201-4 (0.20 g, 0.46 mmol) in DCM (5 mL) was added TFA (1 mL). The reaction was stirred at rt for 30 min. The mixture was evaporated to afford the crude product, which was diluted with water (10 mL) and the mixture was adjusted to pH=7-8 by saturated sodium bicarbonate aqueous solution. The mixture was extracted with DCM (20 mL×3). The combined organic layers were washed with brine (50 mL), dried over Na₂SO₄, filtered through a celite pad and the filtrate was concentrated under reduced pressure to give the intermediate 201-5 (139.0 mg, 99.3% yield) as yellow solid, which was used for next step without further workup.

LC-MS(ESI+): m/z 305.0 (M+H)⁺

¹H NMR (400 MHz, CDCl₃) δ=8.80 (s, 1H), 7.13 (s, 1H), 6.99 (s, 1H), 3.89 (s, 3H), 3.58 (s, 2H), 2.37 (s, 3H).

To a solution of intermediate 201-5 (149.1 mg, 0.49 mmol) in dioxane (2 mL) was added Lawesson's Reagent (206.3 mg, 0.51 mmol). The reaction was stirred at 65° C. for 16 h. The mixture was concentrated under reduced pressure to give a residue, which was purified by Prep-TLC (PE/EtOAc=5/1) to give the intermediate 201-6 (85.0 mg, 54.2% yield) as yellow solid.

LC-MS(ESI+): m/z 321.0 (M+H)⁺.

To a solution of intermediate 201-6 (85.0 mg, 0.27 mmol) in THF (5 mL)/Water (2.5 mL) were added K₂CO₃ (92.0 mg, 0.67 mmol) and iodomethane (98.0 mg, 0.69 mmol) at 0° C. The mixture was stirred at rt for 2 h. To the reaction mixture was added water (10 mL) and extracted with EtOAc (10 mL×2). The combined organic layers were washed with brine (20 mL), dried over Na₂SO₄, filtered through a celite pad and the filtrate was concentrated under reduced pressure to give a residue, which was purified by Prep-TLC (PE/EtOAc=5/1) to give the intermediate 201-7 (72.0 mg, 0.22 mmol, 79.8% yield) as yellow solid.

LC-MS(ESI+): m/z 335.0 (M+H)⁺.

To a solution of intermediate 201-7 (69.0 mg, 0.21 mmol) in EtOH (1 mL) were added (1s,3s)-3-amino-1-methylcyclobutan-1-ol hydrochloride (85.0 mg, 0.63 mmol) and TEA (0.09 mL, 0.63 mmol). The mixture was stirred at room temperature for 30 min, then at 60° C. for 16 h. To the cooled reaction mixture was added water (15 mL) and the mixture was extracted with EtOAc (15 mL×3). The combined organic layers were washed with brine (30 mL), dried over Na₂SO₄, filtered through a celite pad and the filtrate was concentrated under reduced pressure to give a residue, which was recrystallized from diethyl ether (10 mL) to give the intermediate 201-8 (50.0 mg, 61.5% yield) as white solid.

LC-MS(ESI+): m/z 388.1 (M+H)⁺.

To a solution of intermediate 201-8 (45.0 mg, 0.12 mmol) in DCM (2 mL) was added tribromoborane (0.36 mL, 0.36 mmol, 1 M in DCM) at −78° C. The reaction was stirred at −78° C. for 3 h. The reaction was quenched with MeOH at −78° C., then the mixture was concentrated under reduced pressure at room temperature to give the crude, which was purified by Prep-HPLC (Column: C18 OBD 10 μm 19*250 mm; Mobile Phase: [0.1% FA in water-ACN]; B %: 5%-10% ACN, 8.7 min) to give the compound 201.

LC-MS(ESI+): m/z 374.1 (M+H)⁺

¹H NMR (400 MHz, DMSO-d₆)=7.32-7.10 (m, 1H), 7.06 (s, 1H), 6.99 (s, 1H), 4.99 (br.s, 1H), 4.02-3.90 (m, 1H), 3.15 (s, 2H), 2.39-2.30 (m, 2H), 2.27 (s, 3H), 2.04-1.92 (m, 2H), 1.26 (s, 3H).

The compounds below were prepared using a synthesis method similar to that described in Compound 201 by substituting the appropriate starting materials, reagents and reaction conditions.

| Compound | |
|---|---|
| No. | Analytical data |
| 202 | LC-MS(ESI+): m/z 402.0 (M + H)⁺. |
| | ¹H NMR: (400 MHz, DMSO-d₆) δ = 10.50 (br. s, 1 H), 7.05 (s, 1 H), 6.98 |
| | (s, 1 H), 6.81-6.59 (m, 1 H), 5.01-4.88 (m, 1 H), 3.96-3.90 (m, 1 H), |
| | 2.36-2.31 (m, 2 H), 2.29 (s, 3 H), 2.06-1.98 (m, 2 H), 1.39 (s, 6 H), 1.26 |
| | (s, 3 H). |
| 203 | LC-MS(ESI+): m/z 348.1 (M + H)⁺. |
| | ¹H NMR (400 MHz, DMSO-d₆) δ = 10.69 (br. s, 1 H), 7.41-7.11 (m, 1 H), |
| | 6.19 (s, 1 H), 5.02 (br. s, 1 H), 4.51 (t, J = 8.4 Hz, 2 H), 3.98-3.90 (m, 1 H), |
| | 3.22 (s, 2 H), 3.05 (t, J = 8.4 Hz, 2 H), 2.45-2.32 (m, 2 H), 2.18 (s, 3 H), |
| | 2.01-1.95 (m, 2 H), 1.26 (s, 3 H). |
| 204 | LC-MS(ESI+): m/z 340.0 (M + H)⁺. |
| | ¹H NMR: (400 MHz, DMSO-d₆) δ = 10.33 (br. s, 1 H), 7.18-7.02 (m, 1 H), |
| | 6.78-6.70 (m, 2 H), 5.00 (br. s, 1 H), 3.95-3.88 (m, 1 H), 3.12 (s, 2 H), |
| | 2.39-2.34 (m, 2 H), 2.18 (s, 3 H), 1.99-1.88 (m, 2 H), 1.26 (s, 3 H). |
| 205 | LC-MS(ESI+): m/z 361.2 (M + H)⁺. |
| | ¹H NMR (400 MHz, CD₃CN) δ = 8.29 (s, 1 H, from HCOOH), 6.22 (s, 1 |
| | H), 4.57 (t, J = 8.8 Hz, 2 H), 4.38-4.29 (m, 1 H), 3.20 (s, 2 H), 3.09 (t, J = |
| | 8.8 Hz, 2 H), 3.01-2.99 (m, 1 H), 2.86-2.78 (m, 2 H), 2.73-2.68 (m, 1 |
| | H), 2.48 (s, 3 H), 2.41 (s, 3 H), 2.00-1.98 (m, 1 H), 1.76-1.71 (m, 3 H). |
| 206 | LC-MS(ESI+): m/z 339.1 (M + H)⁺. |
| | ¹H NMR (400 MHz, CD₃OD) δ = 6.78 (s, 1H), 6.74 (s, 1H), 4.31-4.12 (m, |
| | 1H), 3.28 (s, 2H), 2.71-2.58 (m, 2H), 2.53-2.42 (m, 2H), 2.24 (s, 3H), |
| | 1.52 (s, 3H). |
| 207 | LC-MS(ESI+): m/z 339.0 (M + H)⁺. |
| | ¹H NMR (400 MHz, CD₃OD) δ = 6.77 (s, 1H), 6.73 (s, 1H), 4.54-4.40 (m, |
| | 1H), 3.34 (s, 2H), 2.85-2.73 (m, 2H), 2.35-2.25 (m, 2H), 2.24 (s, 3H), |
| | 1.56 (s, 3H). |

### Example B-1: NLRP3 Inflammasome Assay

### Reagent and Material

| Reagent Name | Article Number |
|---|---|
| 1X Assay Buffer | 250 mol/L HEPES (pH 7.4), Sigma-H3375 |
| | 10 mmol/L KCl, Sigma-P9333 |
| | 5 mmol/L MgCl₂, Sigma-449172 |
| Wash buffer | Same as assay buffer, store at 4° C. |
| NLRP3 cell lysate | Prepared by WuXi AppTec |
| [3H]-MCC950 | Pharmaron-TRQ42137 |
| MCC950 | TOCRIS-5479 |
| Assay/source plate | 96-well plate (Corning-3631) |
| GF/B Plate | PerkinElmer-600517 |
| DMSO | Sigma-472301 |
| Microscint-20 | PerkinElmer-6013621 |

### Assay Procedure

1) Compound dilutions for IC₅₀ test: Starting at 100 μM, 3 fold dilutions were conducted. Next 11 doses were dispensed at 1% DMSO concentration. 9 μL of compound (10 mM stock from compound management team) as added to 384LDV plate. Echo was used for the dilution and transfer the compounds to assay the plate at 100 μM (top concentration at 1% DMSO, 1.25 μL).
2) Radioligand dilution: working concentration was 25 nM [3H]-MCC950. 1 μL [3H]-MCC950 (23 μM stock) was added to 919 μL assay buffer.
3) Prepare and transfer 100 μl of insect cell lysate to a 96-well assay plate. Final concentration: 15 μg/well for IC50 test.
4) Transfer 25 μL of diluted ligand to the assay plate.
5) Cover assay plates with foil tape and incubate 1.5 hour, at room temperature with gentle shaking.
6) Using the Packard Harvester, filter the wells of the assay plate through the wells of the GF/B filter plates. Wash 8 times with cold washing buffer (4° C., 0.4 mL per well per wash).
7) Place the filter plates in an oven for 0.5 hours at 50° C.
8) Seal the bottom of the dry filter plates with backing tape. Dispense 50 μL Microscint-20 to each well of the filter plate and cover GF/B plate with TopSeal-A.
9) Microbeta was used to count the signal.
10) Microbeta Setting: Count Time was 30 seconds per well.

### Example B-2: NLRP3 Inflammasome Activation Assay on Human Monocytes

Day 1: Isolate human monocytes from PBMC

Isolate monocytes from PBMC using human pan monocytes isolation kit and a LS Column. Resuspend monocytes in RPMI 1640 medium and count cells, then seed monocytes in 96-well plates and incubate at 37° C., 5% CO₂ overnight.
Day 2: Stimulate cells with LPS & ATP
1. Remove culture medium and pre-treat monocytes by adding different concentrations of compounds or DMSO as a control to corresponding wells and incubate at 37° C., 5% CO₂. Compounds and DMSO are diluted using serum free RPMI 1640 medium.
2. Add serum-free RPMI 1640 medium containing LPS to all wells, then incubate cells for some time at 37° C., 5% CO₂.
3. At the end of incubation, the cells were stimulated with ATP for some time except the cells in negative control wells. Transfer the supernatants to new plates and store at −80° C.
Day 3: Run ELISA

Run Elisa according to the procedures from BD Biosciences.

### Example B-3: IL-1β Release THP-1 Assay

THP-1 cells Culture RPMI1640 medium, 10% FBS, 1% PS, 55 uM β-Mer at 37° C. & 5% CO₂ incubator.

IL-1β Release Detection
1) Seed THP-1 in complete RPMI 1640 medium containing PMA into 96 well plate coated with poly-L-lysin incubate for 24 hours.
2) Remove medium, wash the differentiated THP-1 cells with PBS, add FBS free RPMI 1640 medium.
3) Add LPS and incubate for 3 hours at 37° C. & 5% CO₂ incubator.
4) Add compounds and incubate for 30 minutes at 37° C. & 5% CO₂ incubator.
5) Add Nigericin and incubate for 1 hour at 37° C. & 5% CO₂ incubator.
6) Collect supernatant to test IL-1β by Elisa.

### Data Analysis

a) Assay robustness check with DMSO and Low control data:

$$H = Ave ( DMSO )$$

$$L = Ave ( Low control )$$

$$SD ( H ) = STDEV ( DMSO )$$

$$SD ( L ) = STDEV ( Low control )$$

$$CV \% ( H ) = 100 * ( SD\_H / Ave\_H )$$

$$CV \% ( L ) = 100 * SD\_L / Ave\_L$$

$${Z}^{′} = 1 - 3 * ( SD\_H + SD\_L ) / ( Ave\_H - Ave\_L )$$

$$change \% = Sample / Ave\_L * 100$$

b) Fit the cpd IC50 from non-linear regression equation:

$$Y = Bottom + ( Top - Bottom ) / ( 1 + 1 {0}^{⋀} ( ( LogIC 50 - X ) * HillSlope ) )$$

X: cpd concentration
Y: change %
Top and Bottom: Plateaus in same units as Y
logIC50: same log units as X
HillSlope: Slope factor or Hill slope

The THP-1 IL-1β IC₅₀ data for select compounds are shown in Table 3.

### Example B-4: NLRP3 Enzymatic Activity ADP-Glo Assay

NLRP3 activity test experiment, measuring the hydrolysis of NLRP3 on the substrate ATP using the ADP-Glo assay. First, after adding an inhibitor containing 0.5% DMSO with Echo, add 5 μL of NLRP3 (ICE, YM2306T-H06MHS) enzyme solution to each well, centrifuge at 1000 rpm for 1 min at room temperature and react for 10 min. Then add 5 μL of ATP (Promega, V915A) substrate solution to each well for 90 min at room temperature; NLRP3 and ATP were prepared in 50 mM HEPES, 10 mM MgCl2, 0.01% Brij-35, 1 mM EGTA, and 2 mM DTT buffers at final concentrations of 15 nM and 1 μM, respectively. Subsequently, 10 μL ADP-Glo reagent solution (Promega, V9102) was added into each assay well, centrifuged at 1000 rpm for 1 min, and then incubated at room temperature for 45 min. Finally, 20 μL ADP-Glo detection solution (Promega, V9102) is added to each well, centrifuged at 1000 rpm for 1 min and reacted for 45 min at room temperature. The luminescence signal values were read using a BMG instrument and the IC50 values were determined by fitting the data to an S-shaped dose-response curve using nonlinear regression. The Enzymatic ADP-Glo Assay data for select compounds are shown in Table 3.

### Example B-5: hERG Screening Assay Using Electrophysiological Manual Patch-Clamp

Cell lines: hERG-CHO cells

Method: CHO cells stably expressing the transcript of hERG were investigated by whole-cell manual patch clamp technique. hERG-CHO cells were cultured in 35 mm dishes to a maximum 70-80% confluence at 37° C. 5% CO₂ incubator. The culture media (F12 medium (Invitrogen 11765062, ThermoFisher, USA) supplemented with 10% fetal bovine serum (Invitrogen 10099141, ThermoFisher, USA), 100 μg/mL G418 (Invitrogen 11811023, ThermoFisher, USA) and 100 μg/mL Hygromycin B (Invitrogen 10687010, ThermoFisher, USA)) was removed and the hERG-CHO cells were washed with extracellular solution (in mM): 140 NaCl, 5 KCl, 1 CaCl₂), 1.25 MgCl₂, 10 HEPES and 10 Glucose, pH 7.4 with NaOH. Then the cells were dissociated with 0.25% Trypsin-EDTA for 3-5 min, after that, the digestion solution was removed and the cells were resuspended in the extracellular solution with pipette by pipetting up and down several times. Resuspended cells were transferred to dishes for recording and cells were perfused with extracellular solution. Electrodes (a tip resistance of 3-5 MegOhm) were pulled from borosilicate grass pipette (Sutter instrument BF150-86-10) and filled with intracellular solution (in mM): 140 KCl, 1 MgCl₂, 1 CaCl₂), 10 EGTA and 10 HEPES, pH 7.2 with KOH. Data were obtained using a patch-clamp amplifier, signals were filtered at 2 kHz and sampled at frequencies of 10 kHz using pClamp 10 software. The cells were held at −100 mV and hERG potassium currents were activated by a 2 s depolarization potential of +20 mV followed by a repolarization potential of −50 mV for Is then back to the holding potential. Experiments were performed at room temperature.

Data analysis: Data were retrieved and analyzed using pClamp 10, GraphPad Prism 8 and Excel software. The peak amplitude of hERG currents were measured using clampfit and exported to Excel and GraphPad Prism 8 for subsequent analysis. The concentrations of compounds to yield 50% block of the currents (IC₅₀) were obtained by fitting normalized concentration-inhibition relationships to the equation in Prism 8 software as below:

$$Y = Bottom + ( Top - Bottom ) / ( 1 + 1 {0}^{⋀} ( ( {LogIC}_{50} - X ) * HillSlope ) )$$

where Y is the inhibition % corresponding to X, [X] is the Log value of concentration of compound in the external solution and HillSlope is the Hill coefficient. The ratio of inhibition was calculated by using the equation: Inhibition=(1−I/Io)*100%, where Io and I are the current amplitude measured in control and in the presence of compounds, respectively. n is not less than 2 cells for each concentration.

Results: Compounds IC₅₀ on hERG and concentration-response curves

The hERG inhibition data for select compounds are shown in Table 4.

TABLE 3
Enzymatic Activity ADP-Glo Assay IC₅₀ (nM):
0 < A ≤ 10; 10 < B ≤ 100; 100 < C ≤ 1000; 1000 < D.
THP-1 IL-1β IC₅₀ (nM):
0 < A ≤ 10; 10 < B ≤ 100; 100 < C ≤ 1000; 1000 < D.

| | NLRP3_ADP-Glo_IC50 | Potency, THP-1 IL-1β, IC50 (nM), |
|---|---|---|
| Compound No. | (nM), Example B-4 | Example B-3 |
| 1 | B | A |
| 2 | | A |
| 3 | | A |
| 5 | | C |
| 6 | | A |
| 7 | | B |
| 8 | B | B |
| 9 | | B |
| 10 | B | B |
| 11 | | B |
| 16 | | B |
| 17 | | B |
| 20 | | B |
| 21 | | B |
| 22 | | C |
| 23 | B | A |
| 24 | | C |
| 25 | B | B |
| 26 | | C |
| 28 | B | A |
| 29 | | B |
| 30 | B | B |
| 31 | B | B |
| 32 | D | |
| 33 | D | |
| 34 | D | |
| 35 | B | B |
| 36 | C | C |
| 37 | B | B |
| 39 | C | |
| 40 | B | B |
| 41 | C | |
| 42 | D | |
| 43 | D | |
| 45 | B | B |
| 46 | A | A |
| 47 | A | A |
| 48 | B | C |
| 49 | D | |
| 50 | B | B |
| 51 | B | A |
| 52 | D | |
| 54 | A | |
| 55 | C | |
| 56 | C | |
| 57 | B | B |
| 58 | B | B |
| 59 | A | A |
| 60 | B | B |
| 61 | B | |
| 62 | C | |
| 63 | C | |
| 64 | B | |
| 65 | C | |
| 66 | B | B |
| 67 | B | B |
| 68 | B | B |
| 69 | C | |
| 70 | C | |
| 71 | B | |
| 72 | A | |
| 201 | B | B |
| 202 | B | B |
| 203 | B | B |
| 204 | B | A |
| 205 | A | A |
| 206 | B | B |
| 207 | C | |

TABLE 4
hERG inhibition

| | hERG inhibition_IC50 |
|---|---|
| Compound No. | (μM), Example B-5 |
| 8 | 19.1 |
| 10 | >30 |
| 28 | 29.4 |
| 31 | >30 |
| 59 | >30 |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A compound having the structure of Formula (Ia), or a pharmaceutically acceptable salt or a stereoisomer thereof: 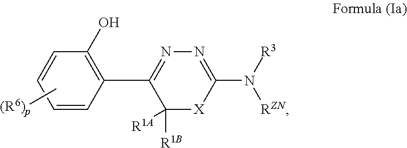
wherein;
X is —S—;
R^{1A} is hydrogen or methyl;
R^{1B} is hydrogen or methyl;
R³ is C₃-C₁₂cycloalkyl, or 4 to 12 membered heterocycloalkyl; each of which is optionally substituted with 1, 2, or 3 R⁸;
R⁸ is each independently selected from halogen, —OH, —OR^{a}, —SH, —SR^{a}, SF₅, —NR^{c}R^{d}, C₁-C₆alkyl, C₁-C₆haloalkyl, C₁-C₆hydroxyalkyl, C₁-C₆heteroalkyl, C₁-C₆aminoalkyl, and C₃-C₆cycloalkyl;
R^{ZN} is hydrogen, C₁-C₆alkyl, or C₁-C₆haloalkyl;
each R⁶ is independently halogen, —OH, C₁-C₆alkyl, C₁-C₆haloalkyl, or C₁-C₆hydroxyalkyl;
or two R⁶ are taken together with the atoms to which they are attached to form a C₄-C₈ cycloalkyl or 5 to 6-membered heterocycloalkyl, each of which is optionally substituted with one or more R¹²;
each R¹² is independently halogen, —OH, —CN, —NO₂, —OR^{a}, —NR^{c}R^{d}, C₁-C₆alkyl, C₁-C₆alkoxy, C₁-C₆haloalkyl, C₁-C₆hydroxyalkyl, or C₁-C₆aminoalkyl;
each R^{a} is independently C₁-C₆alkyl or C₁-C₆haloalkyl;
each R^{c} and R^{d} are independently hydrogen, C₁-C₆alkyl or C₁-C₆haloalkyl; and
p is 1, 2, 3, or 4.

2. The compound of claim 1, or a pharmaceutically acceptable salt or a stereoisomer thereof, wherein R³ is

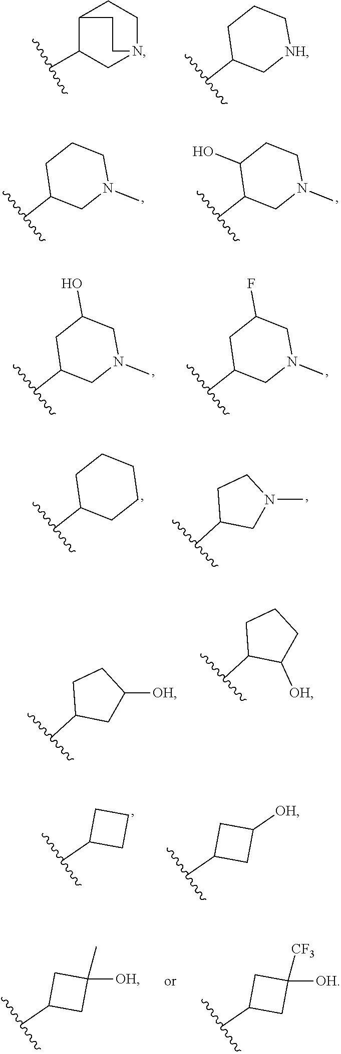

3. The compound of claim 2, or a pharmaceutically acceptable salt or a stereoisomer thereof, wherein two R⁶ are taken together with the atoms to which they are attached to form a C₄-C₈ cycloalkyl, which is optionally substituted with one or more R¹².

4. The compound of claim 2, or a pharmaceutically acceptable salt or a stereoisomer thereof, wherein two R⁶ are taken together to form a 5 to 6-membered heterocycloalkyl, which is optionally substituted with one or more R¹².

5. The compound of claim 4, or a pharmaceutically acceptable salt or a stereoisomer thereof, wherein 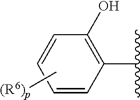
is

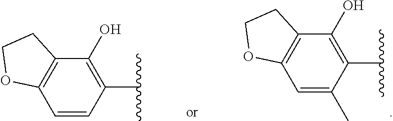 [SMILES: *c1ccc2c(c1O)CCO2 ; *c1c(C)cc2c(c1O)CCO2]

6. The compound of claim 1, or a pharmaceutically acceptable salt or stereoisomer thereof, wherein R³ is

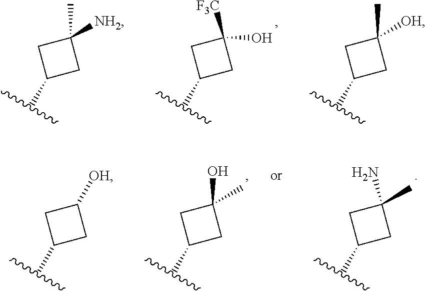

7. The compound of claim 6, or a pharmaceutically acceptable salt or a stereoisomer thereof, wherein two R⁶ are taken together with the atoms to which they are attached to form a C₄-C₈ cycloalkyl, which is optionally substituted with one or more R¹².

8. The compound of claim 6, or a pharmaceutically acceptable salt or a stereoisomer thereof, wherein two R⁶ are taken together to form a 5 to 6-membered heterocycloalkyl, which is optionally substituted with one or more R¹².

9. The compound of claim 8, or a pharmaceutically acceptable salt or a stereoisomer thereof, wherein 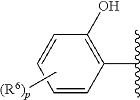
is

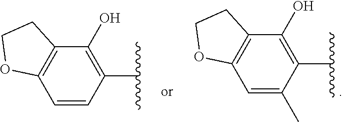 [SMILES: *c1ccc2c(c1O)CCO2 ; *c1c(C)cc2c(c1O)CCO2]

10. The compound of claim 1, or a pharmaceutically acceptable salt or a stereoisomer thereof, wherein R^{ZN} is hydrogen.

11. The compound of claim 1, or a pharmaceutically acceptable salt or a stereoisomer thereof, wherein the compound is 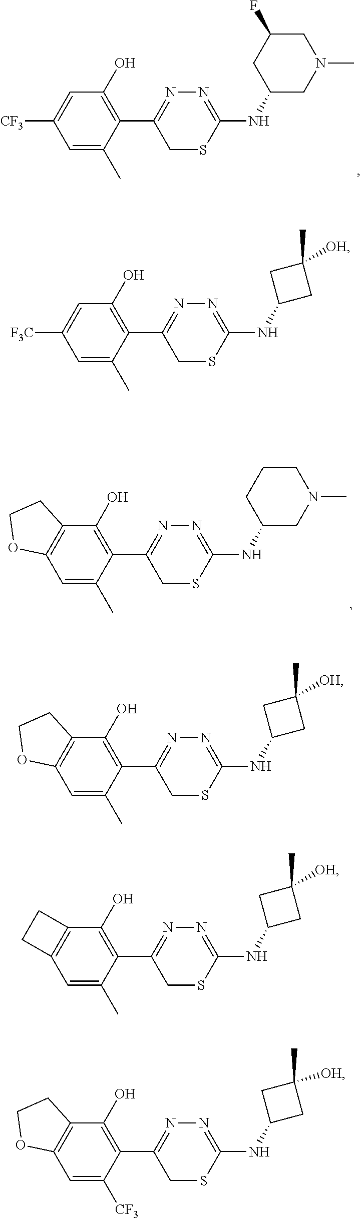 [SMILES: Cc1cc(C(F)(F)F)cc(O)c1C1=NN=C(N[C@@H]2C[C@@H](F)CN(C)C2)SC1 ; Cc1cc(C(F)(F)F)cc(O)c1C1=NN=C(N[C@H]2C[C@@](C)(O)C2)SC1 ; Cc1cc2c(c(O)c1C1=NN=C(N[C@@H]3CCCN(C)C3)SC1)CCO2 ; Cc1cc2c(c(O)c1C1=NN=C(N[C@H]3C[C@@](C)(O)C3)SC1)CCO2 ; Cc1cc2c(c(O)c1C1=NN=C(N[C@H]3C[C@@](C)(O)C3)SC1)CC2 ; C[C@]1(O)C[C@@H](NC2=NN=C(c3c(C(F)(F)F)cc4c(c3O)CCO4)CS2)C1] 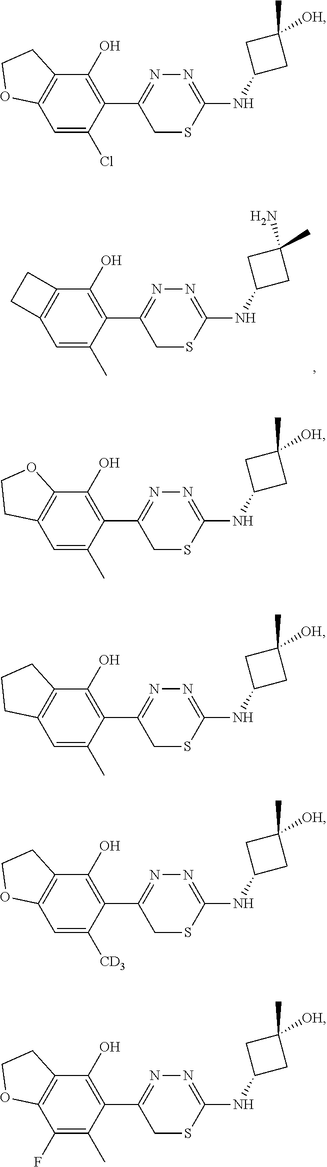 [SMILES: C[C@]1(O)C[C@@H](NC2=NN=C(c3c(Cl)cc4c(c3O)CCO4)CS2)C1 ; Cc1cc2c(c(O)c1C1=NN=C(N[C@H]3C[C@@](C)(N)C3)SC1)CC2 ; Cc1cc2c(c(O)c1C1=NN=C(N[C@H]3C[C@@](C)(O)C3)SC1)OCC2 ; Cc1cc2c(c(O)c1C1=NN=C(N[C@H]3C[C@@](C)(O)C3)SC1)CCC2 ; [2H]C([2H])([2H])c1cc2c(c(O)c1C1=NN=C(N[C@H]3C[C@@](C)(O)C3)SC1)CCO2 ; Cc1c(F)c2c(c(O)c1C1=NN=C(N[C@H]3C[C@@](C)(O)C3)SC1)CCO2] 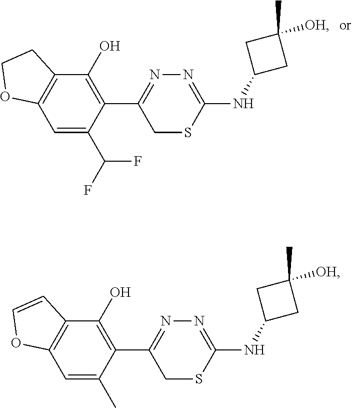 [SMILES: C[C@]1(O)C[C@@H](NC2=NN=C(c3c(C(F)F)cc4c(c3O)CCO4)CS2)C1 ; "or" ; Cc1cc2occc2c(O)c1C1=NN=C(N[C@H]2C[C@@](C)(O)C2)SC1]
or a pharmaceutically acceptable salt thereof.

12. The compound of claim 1, or a pharmaceutically acceptable salt or a stereoisomer thereof, wherein the compound is 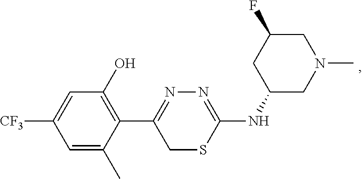 [SMILES: Cc1cc(C(F)(F)F)cc(O)c1C1=NN=C(N[C@@H]2C[C@@H](F)CN(C)C2)SC1]
or a pharmaceutically acceptable salt thereof.

13. The compound of claim 1, or a pharmaceutically acceptable salt or a stereoisomer thereof, wherein the compound is 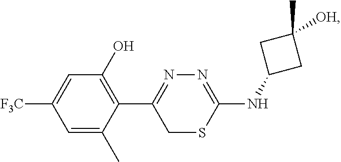 [SMILES: Cc1cc(C(F)(F)F)cc(O)c1C1=NN=C(N[C@H]2C[C@@](C)(O)C2)SC1]
or a pharmaceutically acceptable salt thereof.

14. The compound of claim 1, or a pharmaceutically acceptable salt or a stereoisomer thereof, wherein the compound is 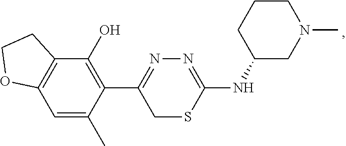 [SMILES: Cc1cc2c(c(O)c1C1=NN=C(N[C@@H]3CCCN(C)C3)SC1)CCO2]
or a pharmaceutically acceptable salt thereof.

15. The compound of claim 1, or a pharmaceutically acceptable salt or a stereoisomer thereof, wherein the compound is 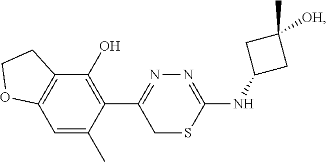 [SMILES: Cc1cc2c(c(O)c1C1=NN=C(N[C@H]3C[C@@](C)(O)C3)SC1)CCO2]
or a pharmaceutically acceptable salt thereof.

16. The compound of claim 1, or a pharmaceutically acceptable salt or a stereoisomer thereof, wherein the compound is 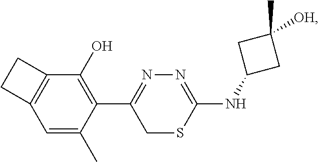 [SMILES: Cc1cc2c(c(O)c1C1=NN=C(N[C@H]3C[C@@](C)(O)C3)SC1)CC2]
or a pharmaceutically acceptable salt thereof.

17. The compound of claim 1, or a pharmaceutically acceptable salt or a stereoisomer thereof, wherein the compound is 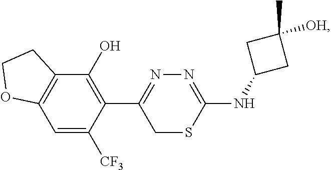 [SMILES: C[C@]1(O)C[C@@H](NC2=NN=C(c3c(C(F)(F)F)cc4c(c3O)CCO4)CS2)C1]
or a pharmaceutically acceptable salt thereof.

18. The compound of claim 1, or a pharmaceutically acceptable salt or a stereoisomer thereof, wherein the compound is 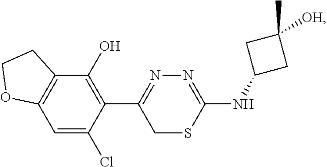 [SMILES: C[C@]1(O)C[C@@H](NC2=NN=C(c3c(Cl)cc4c(c3O)CCO4)CS2)C1]
or a pharmaceutically acceptable salt thereof.

19. The compound of claim 1, or a pharmaceutically acceptable salt or a stereoisomer thereof, wherein the compound is 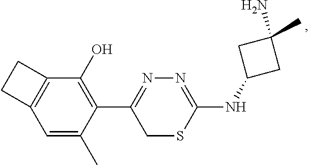 [SMILES: Cc1cc2c(c(O)c1C1=NN=C(N[C@H]3C[C@@](C)(N)C3)SC1)CC2]
or a pharmaceutically acceptable salt thereof.

20. The compound of claim 1, or a pharmaceutically acceptable salt or a stereoisomer thereof, wherein the compound is 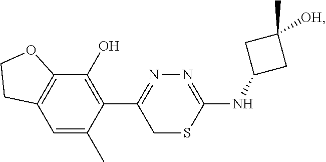 [SMILES: Cc1cc2c(c(O)c1C1=NN=C(N[C@H]3C[C@@](C)(O)C3)SC1)OCC2]
or a pharmaceutically acceptable salt thereof.

21. The compound of claim 1, or a pharmaceutically acceptable salt or a stereoisomer thereof, wherein the compound is 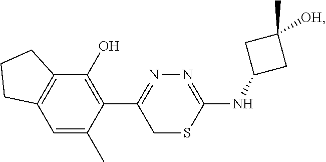 [SMILES: Cc1cc2c(c(O)c1C1=NN=C(N[C@H]3C[C@@](C)(O)C3)SC1)CCC2]
or a pharmaceutically acceptable salt thereof.

22. The compound of claim 1, or a pharmaceutically acceptable salt or a stereoisomer thereof, wherein the compound is 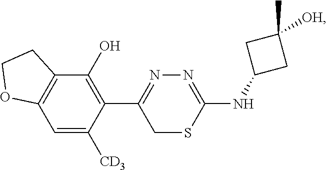 [SMILES: [2H]C([2H])([2H])c1cc2c(c(O)c1C1=NN=C(N[C@H]3C[C@@](C)(O)C3)SC1)CCO2]
or a pharmaceutically acceptable salt thereof.

23. The compound of claim 1, or a pharmaceutically acceptable salt or a stereoisomer thereof, wherein the compound is 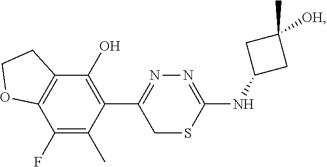 [SMILES: Cc1c(F)c2c(c(O)c1C1=NN=C(N[C@H]3C[C@@](C)(O)C3)SC1)CCO2]
or a pharmaceutically acceptable salt thereof.

24. The compound of claim 1, or a pharmaceutically acceptable salt or a stereoisomer thereof, wherein the compound is 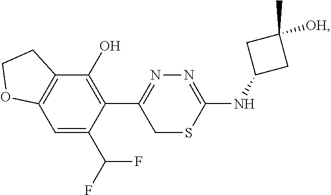 [SMILES: C[C@]1(O)C[C@@H](NC2=NN=C(c3c(C(F)F)cc4c(c3O)CCO4)CS2)C1]
or a pharmaceutically acceptable salt thereof.

25. The compound of claim 1, or a pharmaceutically acceptable salt or a stereoisomer thereof, wherein the compound is 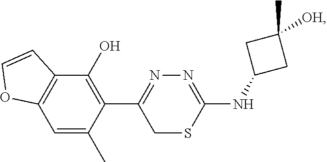 [SMILES: Cc1cc2occc2c(O)c1C1=NN=C(N[C@H]2C[C@@](C)(O)C2)SC1]
or a pharmaceutically acceptable salt thereof.

26. A pharmaceutical composition comprising a compound of claim 11, or a pharmaceutically acceptable salt or a stereoisomer thereof, and at least one pharmaceutically acceptable excipient.

27. A method of treating a disease or disorder in which the NLRP3 signaling contributes to the pathology, and/or symptoms, and/or progression, of the disease or disorder, comprising administering a therapeutically effective amount of a compound of claim 11, or a pharmaceutically acceptable salt or a stereoisomer thereof.

28. The method of claim 27, wherein the disease or disorder is a neurodegenerative disease.
